(19) United States
(12) Patent Application Publication
(10) Pub. No.: US 2024/0327799 A1
(43) Pub. Date: Oct. 3, 2024

(54) COMPOSITIONS AND METHODS FOR CELLULAR REPROGRAMMING USING CIRCULAR RNA

(71) Applicant: ElevateBio Technologies, Inc., Waltham, MA (US)

(72) Inventors: Melissa CARPENTER, Black Diamond, WA (US); Santosh NARAYAN, Cambridge, MA (US); Austin THIEL, Southborough, MA (US); Yin Miranda YANG, Cambridge, MA (US)

(21) Appl. No.: 18/745,761

(22) Filed: Jun. 17, 2024

Related U.S. Application Data
(63) Continuation of application No. PCT/US2022/081898, filed on Dec. 19, 2022.
(60) Provisional application No. 63/291,645, filed on Dec. 20, 2021.

(51) Int. Cl. C12N 5/074; A61K 35/545; C12N 9/22; C12N 15/11; C12N 15/113; C12N 15/87

(52) U.S. Cl. CPC C12N 5/0696; A61K 35/545; C12N 9/22; C12N 15/111; C12N 15/113; C12N 15/87; C12N 2310/141; C12N 2310/20; C12N 2501/01; C12N 2501/602; C12N 2501/603; C12N 2501/604; C12N 2501/605; C12N 2501/606; C12N 2501/608; C12N 2506/11; C12N 2510/00

(57) ABSTRACT

The present disclosure provides methods of producing iPSCs comprising contacting a cell (e.g., CD34+ cell or other blood cell) in suspension with one or more circular RNAs encoding one or more reprogramming factors and maintaining the cell under conditions under which a reprogrammed iPSC is obtained. In some embodiments, the circular RNA encodes a reprogramming factor (selected from Oct3/4, Klf4, Sox2, Nanog, Lin28, c-Myc, or L-Myc, or a fragment or variant thereof.

### CROSS-REFERENCE TO RELATED APPLICATIONS

This application is a Continuation of International Application No. PCT/US2022/081898, filed Dec. 19, 2022, which claims priority to U.S. Provisional Application No. 63/291,645, filed Dec. 20, 2021, the contents of each of which is herein incorporated by reference in its entirety.

### DESCRIPTION OF THE TEXT FILE SUBMITTED ELECTRONICALLY

The contents of the text file submitted electronically herewith are incorporated by reference in their entirety: a computer readable format copy of the Sequence Listing (filename: ELVT_015_01US_SeqList_ST26.xml; Size: 52,026 bytes; and Date of Creation: Jun. 17, 2024) is herein incorporated by reference in its entirety.

### BACKGROUND

Induced pluripotent stem cells (iPSCs) have transformed drug discovery and healthcare. iPSCs are generated by reprogramming somatic cells back into an embryonic-like pluripotent state that enables the development of various human cell types needed for research and/or therapeutic purposes.

iPSCs are typically derived by introducing one or more reprogramming factors (e.g., Oct3/4, Klf4, Sox2, Nanog, Lin28, c-Myc, and/or L-Myc) into a somatic cell. Although reprogramming factors can be introduced into a cell using standard approaches, these approaches suffer from various drawbacks. For example, self-replicating RNA systems use RNA replicons that are able to self-replicate. The nature of such replicating vectors poses a risk of genome integration. mRNA-based reprogramming is laborious and involves multiple transfections of mRNA due to fast turnover of mRNA molecules. Exogenous mRNA is also immunogenic, which necessitates the use of immune evasion factors (e.g., inhibitors of interferon pathways) and/or modified nucleotides to minimize toxicity.

Accordingly, there is a need in the art for improved compositions and methods for producing iPSCs.

### BRIEF SUMMARY

In some embodiments, the present disclosure provides a method of producing an induced pluripotent stem cell (iPSC), the method comprising contacting a blood cell in suspension with a circular RNA encoding at least one reprogramming factor selected from Oct3/4, Klf4, Sox2, Nanog, Lin28, c-Myc, or L-Myc, or a fragment or variant thereof, and maintaining the cell under conditions under which the iPSC is obtained. In some embodiments, the blood cell is selected from a T cell, a B cell, a natural killer (NK) cell, a natural killer T (NKT) cell, a peripheral blood mononuclear cell (PBMC), and a cord blood mononuclear cell (CBMC). In some embodiments, the blood cell is selected from a T cell and an NK cell.

In some embodiments, the present disclosure provides a method of producing an induced pluripotent stem cell (iPSC), the method comprising contacting a CD34+ cell in suspension with a circular RNA encoding at least one reprogramming factor selected from Oct3/4, Klf4, Sox2, Nanog, Lin28, c-Myc, or L-Myc, or a fragment or variant thereof, and maintaining the cell under conditions under which the iPSC is obtained.

In some embodiments, the at least one reprogramming factor is a human or a humanized reprogramming factor. In some embodiments, the at least one reprogramming factor is Oct3/4, and wherein the Oct3/4 has the amino acid sequence of SEQ ID NO: 7, or a sequence at least 90% or at least 95% identical thereto. In some embodiments, the circular RNA comprises a nucleic acid sequence of SEQ ID NO: 8, or a sequence at least 90% or at least 95% identical thereto.

In some embodiments, the at least one reprogramming factor is Klf4, and wherein the Klf4 has the amino acid sequence of SEQ ID NO: 9 or 10, or a sequence at least 90% or at least 95% identical thereto. In some embodiments, the circular RNA comprises a nucleic acid sequence of SEQ ID NO: 11, or a sequence at least 90% or at least 95% identical thereto.

In some embodiments, the at least one reprogramming factor is Sox2, and wherein Sox2 has the amino acid sequence of SEQ ID NO: 12, or a sequence at least 90% or at least 95% identical thereto. In some embodiments, the circular RNA comprises a nucleic acid sequence of SEQ ID NO: 13, or a sequence at least 90% or at least 95% identical thereto.

In some embodiments, the at least one reprogramming factor is Nanog, and wherein the Nanog has the amino acid sequence of SEQ ID NO: 14 or 15, or a sequence at least 90% or at least 95% identical thereto. In some embodiments, the circular RNA comprises a nucleic acid sequence of SEQ ID NO: 16, or a sequence at least 90% or at least 95% identical thereto.

In some embodiments, the at least one reprogramming factor is Lin28A, and wherein the Lin28A has the amino acid sequence of SEQ ID NO: 17, or a sequence at least 90% or at least 95% identical thereto. In some embodiments, the circular RNA comprises a nucleic acid sequence of SEQ ID NO: 18, or a sequence at least 90% or at least 95% identical thereto.

In some embodiments, the at least one reprogramming factor is c-Myc, and wherein the c-Myc has the amino acid sequence of SEQ ID NO: 19 or 20, or a sequence at least 90% or at least 95% identical thereto. In some embodiments, the circular RNA comprises a nucleic acid sequence of SEQ ID NO: 21, or a sequence at least 90% or at least 95% identical thereto.

In some embodiments, the at least one reprogramming factor is L-Myc, and wherein the L-Myc has the amino acid sequence of any one of SEQ ID NO: 22-24, or a sequence at least 90% or at least 95% identical thereto. In some embodiments, the circular RNA comprises a nucleic acid sequence of SEQ ID NO: 25, or a sequence at least 90% or at least 95% identical thereto.

In some embodiments, the circular RNA is substantially non-immunogenic. In some embodiments, the circular RNA comprises one or more M-6-methyladenosine (m6A) residues. In some embodiments, the circular RNA comprises from about 200 nucleotides to about 5,000 nucleotides.

In some embodiments, the circular RNA comprises an internal ribosome entry site (IRES) operably linked to the protein-coding sequence. In some embodiments, the circular RNA encodes two or more reprogramming factors selected from Oct3/4, Klf4, Sox2, Nanog, Lin28, c-Myc, or L-Myc.

In some embodiments, the method comprises contacting the cell with a first circular RNA encoding Oct4 or a fragment or variant thereof, a second circular RNA encoding Sox2 or a fragment or variant thereof, a third circular RNA encoding Klf4 or a fragment or variant thereof, a fourth circular RNA encoding C-Myc or a fragment or variant thereof, a fifth circular RNA encoding Lin28 or a fragment or variant thereof, and a sixth circular RNA encoding Nanog or a fragment or variant thereof.

In some embodiments, the method comprises contacting the cells with one or more circular RNAs encoding one or more of a group of reprogramming factors consisting of Oct3/4, Klf4, Sox2, Nanog, Lin28, and c-Myc, or fragments or variants thereof.

In some embodiments, the present disclosure provides a method of producing an induced pluripotent stem cell (iPSC), the method comprising contacting a blood cell or a CD34+ cell in suspension with six circular RNAs consisting of a first circular RNA encoding Oct4, a second circular RNA encoding Sox2, a third circular RNA encoding Klf4, a fourth circular RNA encoding C-Myc, a fifth circular RNA encoding Lin28, and a sixth circular RNA encoding Nanog, and maintaining the cell under conditions under which the iPSC is obtained.

In some embodiments, the present disclosure provides a method of producing an induced pluripotent stem cell (iPSC), the method comprising contacting a blood cell or a CD34+ cell in suspension with three circular RNAs consisting of a first circular RNA encoding Oct4, a second circular RNA encoding Sox2, and a third circular RNA encoding Klf4, and maintaining the cell under conditions under which the iPSC is obtained. In some embodiments, the method does not comprise contacting the CD34+ cell with a circular RNA encoding any of Nanog, Lin28, and c-Myc.

In some embodiments, the present disclosure provides a method of producing an induced pluripotent stem cell (iPSC), the method comprising contacting a blood cell or a CD34+ cell in suspension with three circular RNAs consisting of a first circular RNA encoding Oct4, a second circular RNA encoding Sox2, and a third circular RNA encoding Klf4, and a fourth circular RNA encoding C-Myc, and maintaining the cell under conditions under which the iPSC is obtained. In some embodiments, the method does not comprise contacting the CD34+ cell with a circular RNA encoding either of Nanog or Lin28.

In some embodiments, the present disclosure provides a method of producing an induced pluripotent stem cell (iPSC), the method comprising contacting a blood cell or a CD34+ cell in suspension with three circular RNAs consisting of a first circular RNA encoding Oct4, a second circular RNA encoding Sox2, and a third circular RNA encoding Klf4, and a fourth circular RNA encoding Nanog, and a fifth circular RNA encoding Lin 28, and maintaining the cell under conditions under which the iPSC is obtained. In some embodiments, the method does not comprise contacting the CD34+ cell with a circular RNA encoding c-Myc.

In some embodiments, the cell is not contacted with any factor selected from E3, K3, B18R. In some embodiments, the cell is not contacted with any micro RNAs (miRs). In some embodiments, the cell is not contacted with one or more factor selected from E3, K3, B18R, one or more micro RNAs (miRs), or a combination thereof. In some embodiments, the miRs comprise miR302a, miR302b, miR302c, miR302d, and miR367.

In some embodiments, the cell is directly contacted with the at least one circular RNA. In some embodiments, the cell is contacted with each of the at least one circular RNA once. In some embodiments, the method comprises contacting the cell with each of the at least one circular RNA two, three, four, or more times. In some embodiments, the method comprises contacting the cell with each of the at least one circular RNA fewer than four times. In some embodiments, the method comprises contacting the cell with each of the at least one circular RNA from 2 to 4 times.

In some embodiments, the concentration of each of the at least one circular RNAs is at least 3 pg RNA/cell. In some embodiments, the concentration of each of the at least one circular RNAs is from about 5 pg RNA/cell to about 15 pg RNA/cell.

In some embodiments, the contacting the cell is performed by electroporation.

In some embodiments, the method comprises further contacting the cell with an RNA polynucleotide encoding one or more viral proteins selected from E3, K3, or B18R. In some embodiments, (a) the B18R has a sequence of SEQ ID NO: 26, or a sequence at least 90% or at least 95% identical thereto; (b) the E3 has a sequence of SEQ ID NO: 27, or a sequence at least 90% or at least 95% identical thereto; and/or (c) the K3 has a sequence of SEQ ID NO: 28, or a sequence at least 90% or at least 95% identical thereto. In some embodiments, the method comprises further contacting the cell with one or more microRNAs (miRs). In some embodiments, the miRs are selected from miR302a, miR302b, miR302c, miR302d, and miR367.

In some embodiments, the method results in one or more of: (i) an increase in the number of reprogrammed iPSC present at the end of culture compared to a method of producing an iPSC with one or more linear RNAs; and/or (ii) a decrease in cell death at one or more timepoints during reprogramming compared to a method of producing an iPSC with one or more linear RNAs. In some embodiments, the method results in each of: (i) an increase in the number of reprogrammed iPSC present at the end of culture compared to a method of producing an iPSC with one or more linear RNAs; and (ii) a decrease in cell death at one or more timepoints during reprogramming compared to a method of producing an iPSC with one or more linear RNAs.

In some embodiments, the present disclosure provides an iPSC produced by the methods described herein. In some embodiments, the present disclosure provides a differentiated cell derived from an iPSC described herein. In some embodiments, the differentiated cell is a muscle cell, a neural cell, a cell of the central nervous system, an ocular cell, a chondrocyte, an osteocyte, a tendon cell, a renal cell, a cardiomyocyte, a hepatocyte, an islet cell, a keratinocyte, a T-cell, or a NK-cell.

In some embodiments, the present disclosure provides a method for reprogramming and editing the genome of a cell, the method comprising: (i) contacting the cell with a recombinant circular RNA comprising a protein-coding sequence, wherein the protein-coding sequence encodes at least one reprogramming factor, and (ii) contacting the cell with an enzyme capable of editing the DNA or RNA of the cell, or a nucleic acid encoding the same.

In some embodiments, the present disclosure provides a method for reprogramming and editing the genome of a cell, the method comprising simultaneously contacting the cell with: (i) a recombinant circular RNA comprising a protein-coding sequence, wherein the protein-coding sequence encodes at least one reprogramming factor, and (ii) an enzyme capable of editing the DNA or RNA of the cell, or a nucleic acid encoding the same.

In some embodiments, the at least one reprogramming factor is a human or a humanized reprogramming factor.

In some embodiments, the at least one reprogramming factor is Oct3/4, and wherein the Oct3/4 has the amino acid sequence of SEQ ID NO: 7, or a sequence at least 90% or at least 95% identical thereto. In some embodiments, the recombinant circular RNA comprises a nucleic acid sequence of SEQ ID NO: 8, or a sequence at least 90% or at least 95% identical thereto.

In some embodiments, the at least one reprogramming factor is Klf4, and wherein the Klf4 has the amino acid sequence of SEQ ID NO: 9 or 10, or a sequence at least 90% or at least 95% identical thereto. In some embodiments, the recombinant circular RNA comprises a nucleic acid sequence of SEQ ID NO: 11, or a sequence at least 90% or at least 95% identical thereto.

In some embodiments, the at least one reprogramming factor is Sox2, and wherein Sox2 has the amino acid sequence of SEQ ID NO: 12, or a sequence at least 90% or at least 95% identical thereto. In some embodiments, the recombinant circular RNA comprises a nucleic acid sequence of SEQ ID NO: 13, or a sequence at least 90% or at least 95% identical thereto.

In some embodiments, the at least one reprogramming factor is Nanog, and wherein the Nanog has the amino acid sequence of SEQ ID NO: 14 or 15, or a sequence at least 90% or at least 95% identical thereto. In some embodiments, the recombinant circular RNA comprises a nucleic acid sequence of SEQ ID NO: 16, or a sequence at least 90% or at least 95% identical thereto.

In some embodiments, the at least one reprogramming factor is Lin28A, and wherein the Lin28A has the amino acid sequence of SEQ ID NO: 17, or a sequence at least 90% or at least 95% identical thereto. In some embodiments, the recombinant circular RNA comprises a nucleic acid sequence of SEQ ID NO: 18, or a sequence at least 90% or at least 95% identical thereto.

In some embodiments, the at least one reprogramming factor is c-Myc, and wherein the c-Myc has the amino acid sequence of SEQ ID NO: 19 or 20, or a sequence at least 90% or at least 95% identical thereto. In some embodiments, the recombinant circular RNA comprises a nucleic acid sequence of SEQ ID NO: 21, or a sequence at least 90% or at least 95% identical thereto.

In some embodiments, the at least one reprogramming factor is L-Myc, and wherein the L-Myc has the amino acid sequence of any one of SEQ ID NO: 22-24, or a sequence at least 90% or at least 95% identical thereto. In some embodiments, the recombinant circular RNA comprises a nucleic acid sequence of SEQ ID NO: 25, or a sequence at least 90% or at least 95% identical thereto.

In some embodiments, the circular RNA is substantially non-immunogenic. In some embodiments, the circular RNA comprises one or more M-6-methyladenosine (m6A) residues. In some embodiments, the circular RNA comprises from about 200 nucleotides to about 5,000 nucleotides.

In some embodiments, the circular RNA comprises an internal ribosome entry site (IRES) operably linked to the protein-coding sequence. In some embodiments, the circular RNA encodes two or more reprogramming factors selected from Oct3/4, Klf4, Sox2, Nanog, Lin28, c-Myc, or L-Myc.

In some embodiments, the method comprises contacting the cell with a first circular RNA encoding Oct4 or a fragment or variant thereof, a second circular RNA encoding Sox2 or a fragment or variant thereof, a third circular RNA encoding Klf4 or a fragment or variant thereof, a fourth circular RNA encoding C-Myc or a fragment or variant thereof, a fifth circular RNA encoding Lin28 or a fragment or variant thereof, and a sixth circular RNA encoding Nanog or a fragment or variant thereof. In some embodiments, the method comprises contacting the cells with one or more circular RNAs encoding Oct3/4, Klf4, Sox2, Nanog, Lin28, c-Myc, and L-Myc, or fragments or variants thereof.

In some embodiments, the cell is not contacted with any ancillary factors selected from E3, K3, B18R, or any micro RNAs (miRs). In some embodiments, the cell is directly contacted with the at least one circular RNA. In some embodiments, the cell is contacted with each of the at least one circular RNAs once. In some embodiments, the method comprises contacting the cell with each of the at least one of the circular RNAs two, three, four, or more times. In some embodiments, the method comprises contacting the cell with each of the at least one circular RNA fewer than four times. In some embodiments, the method comprises contacting the cell with each of the at least one circular RNAs from 2 to 4 times.

In some embodiments, the concentration of the at least one circular RNAs is at least 3 pg RNA/cell. In some embodiments, the concentration of the at least one circular RNAs is from about 5 pg RNA/cell to about 15 pg RNA/cell.

In some embodiments, the contacting the cell is performed by electroporation.

In some embodiments, the method comprises contacting the cell with an RNA polynucleotide encoding one or more viral proteins selected from E3, K3, or B18R. In some embodiments, (a) the B18R has a sequence of SEQ ID NO: 26, or a sequence at least 90% or at least 95% identical thereto; (b) the E3 has a sequence of SEQ ID NO: 27, or a sequence at least 90% or at least 95% identical thereto; and/or (c) the K3 has a sequence of SEQ ID NO: 28, or a sequence at least 90% or at least 95% identical thereto. In some embodiments, the method comprises contacting the cell with an microRNA (miR) selected from miR302a, miR302b, miR302c, miR302d, and miR367.

In some embodiments, the cell is not contacted with any viral proteins selected from E3, K3, B18R, or any micro RNAs (miRs) selected from miR302a, miR302b, miR302c, miR302d, and miR367.

In some embodiments, the enzyme is a transcription activator-like effector nuclease (TALEN), an argonaute endonuclease (NgAgo), a structure-guided endonuclease (SGN), an RNA-guided nuclease (RGN), an Adenosine deaminase acting on RNA (ADAR), or modified or truncated variants thereof. In some embodiments, the RGN is a Cas nuclease selected from Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or a modified or truncated variant thereof. In some embodiments, RGN is a Cas9 nuclease, and the Cas9 nuclease is isolated or derived from S. pyogenes or S. aureus. In some embodiments, the RGN is selected from any one of APG05083.1, APG07433.1, APG07513.1, APG08290.1, APG05459.1, APG04583.1, and APG1688.1, APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, APG07433.1, APG00969, APG03128, APG09748, APG00771, APG02789, APG09106, APG02312, APG07386, APG09980, APG05840, APG05241, APG07280, APG09866, and APG00868.

In some embodiments, the method further comprises contacting the cell with a guide RNA, or a nucleic acid encoding the same.

In some embodiments, the cell is contacted with the circular RNA before it is contacted with the enzyme or the nucleic acid encoding the same. In some embodiments, the cell is contacted with the recombinant circular RNA after it is contacted with the enzyme or the nucleic acid encoding the same. In some embodiments, the cell is contacted with an enzyme capable of editing the DNA or RNA of the cell, or a nucleic acid encoding the same, and a guide RNA, or a nucleic acid encoding the same. In some embodiments, the enzyme is capable of editing the DNA of the cell and wherein the enzyme and the guide RNA are complexed as a ribonucleoprotein prior to contact with the cell. In some embodiments, the contacting the cell is performed by electroporation.

In some embodiments, the present disclosure provides a cell generated by the methods described herein.

In some embodiments, the present disclosure provides a method for reprogramming a cell, the method comprising contacting a cell with one or more circular RNAs encoding six reprogramming factors consisting of Oct3/4, Klf4, Sox2, Nanog, Lin28, and c-Myc. In some embodiments, the method comprises contacting a cell with six circular RNAs each encoding a reprogramming factor from the group consisting of Oct3/4, Klf4, Sox2, Nanog, Lin28, and c-Myc.

In some embodiments, any one of the circular RNA or linear RNAs are conjugated to a lipid nanoparticle. In some embodiments, the cell is not contacted with one or more factors selected from E3, K3, B18R, or one or more micro RNAs (miRs).

In some embodiments, the present disclosure provides a composition comprising an isolated somatic cell comprising one or more circular RNAs encoding a reprogramming factor, wherein the reprogramming factor is selected from the group consisting of Oct3/4, Klf4, Sox2, Nanog, Lin28, and c-Myc.

In some embodiments, the somatic cell comprises one or more circular RNAs, wherein the one or more circular RNAs each encode a reprogramming factor selected from Oct3/4, Klf4, Sox2, Nanog, Lin28, and c-Myc. In some embodiments, the somatic cell comprises six circular RNAs, wherein each circular RNA encodes one of Oct3/4, Klf4, Sox2, Nanog, Lin28, and c-Myc. In some embodiments, the somatic cell comprises five circular RNAs, wherein each circular RNA encodes one of Oct3/4, Klf4, Sox2, Nanog, and Lin28. In some embodiments, the somatic cell comprises four circular RNAs, wherein each circular RNA encodes one of Oct3/4, Klf4, Sox2, and c-Myc. In some embodiments, the somatic cell comprises three circular RNAs, wherein each circular RNA encodes one of Oct3/4, Klf4, and Sox2.

In some embodiments, the present disclosure provides a suspension culture comprising one or more CD34+ cells, wherein the CD34+ cells comprise one or more circRNAs encoding a reprogramming factor. In some embodiments, the CD34+ cells comprise six circRNAs each encoding one reprogramming factor selected from the group consisting of Oct3/4, Klf4, Sox2, Nanog, Lin28, and c-Myc. In some embodiments, the CD34+ cell does not comprise an exogeneous nucleic acid encoding an ancillary factor selected from E3, K3, B18R, or a micro RNAs (miRs).

In some embodiments, the circular RNA is exogenous to the cell.

In some embodiments, the present disclosure provides a composition comprising one or more circular RNAs encoding the reprogramming factors consisting of Oct3/4, Klf4, Sox2, Nanog, Lin28, and c-Myc. In some embodiments, the present disclosure provides a composition comprising two or more circular RNAs encoding the reprogramming factors consisting of Oct3/4, Klf4, Sox2, Nanog, Lin28, and c-Myc. In some embodiments, the present disclosure provides a composition comprising six circular RNAs each encoding one of the reprogramming factors consisting of Oct3/4, Klf4, Sox2, Nanog, Lin28, and c-Myc. In some embodiments, the present disclosure provides a composition comprising five circular RNAs each encoding one of the reprogramming factors consisting of Oct3/4, Klf4, Sox2, Nanog, and Lin28. In some embodiments, the present disclosure provides a composition comprising four circular RNAs each encoding one of the reprogramming factors consisting of Oct3/4, Klf4, Sox2, and c-Myc. In some embodiments, the present disclosure provides a composition comprising three circular RNAs each encoding one of the reprogramming factors consisting of Oct3/4, Klf4, and Sox2.

In some embodiments, the present disclosure provides a kit comprising a composition described herein. In some embodiments, the present disclosure provides a cell comprising a composition described herein. In some embodiments, the cell is a eukaryotic cell. In some embodiments, the cell is a mammalian cell. In some embodiments, the cell is a human cell. In some embodiments, the cell is a CD34+ cell, a T cell, or an NK cell.

In some embodiments, the present disclosure provides a CD34+ cell comprising one or more circular RNAs encoding one or more reprogramming factors selected from Oct3/4, Klf4, Sox2, Nanog, Lin28, and either one of c-Myc, or L-Myc. In some embodiments, the reprogramming factors consist of all six of Oct3/4, Klf4, Sox2, Nanog, Lin28, and c-Myc. In some embodiments, the reprogramming factors consist of all six of Oct3/4, Klf4, Sox2, Nanog, Lin28, and L-Myc. In some embodiments, the reprogramming factors consist of Oct3/4, Klf4, Sox2, Nanog, and Lin28. In some embodiments, the reprogramming factors consist of Oct3/4, Klf4, Sox2, and c-Myc. In some embodiments, the reprogramming factors consist of Oct3/4, Klf4, and Sox2. In some embodiments, the cells exhibit at least one stemness marker selected from SSEA-3, SSEA-4, TRA-1-60, TRA-1-81, TRA-2-49/6E, Alkaline phosphatase, Sox2, E-cadherin, UTF-1, Oct4, Rex1, Nanog, or a combination thereof. In some embodiments, the one or more circular RNAs is exogenous to the cells.

In some embodiments, the CD34+ further comprises one or more genetic modifications. In some embodiments, the one or more genetic modification comprises a gene knockout. In some embodiments, the one or more genetic modification comprises a gene knock-in.

In some embodiments, the present disclosure provides an induced pluripotent stem cell (iPSC) derived from a CD34+ cell of described herein. In some embodiments, the cell is hypoimmunogenic.

In some embodiments, the present disclosure provides a differentiated cell generated from an iPSC described herein.

In some embodiments, the present disclosure provides a method of treating a disease or condition comprising administering to a subject in need thereof an iPSC or the differentiated cell described herein.

In some embodiments, the present disclosure provides a method of transdifferentiating a somatic cell comprising contacting the cell with one or more exogenous circular RNAs. In some embodiments, the present disclosure provides a transdifferentiated cell produced by the methods described herein.

In some embodiments, the present disclosure provides a method of differentiating a cell from an induced pluripotent stem cell (iPSC) comprising contacting the iPSC with one or more circular RNAs. In some embodiments, the present disclosure provides a differentiated cell produced by the methods described herein.

Other objects, advantages and features of the present invention will become apparent from the following specification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing an exemplary protocol for circularizing linear RNA generated using chemical synthesis or in vitro transcription (IVT) to generate circular RNAs. First, linear RNA is prepared. The 5′ end of the linear RNA is then phosphorylated by amplification using primers specific to the flanking sequence. The 5′ and 3′ ends are subsequently ligated using T4 RNA ligase. The circular RNA is purified, or linear side products are denatured enzymatically. The circular RNA may then be contacted with (e.g., transfected into) cells and/or conjugated to a lipid nanoparticle.

FIG. 2A-2G is a schematic showing exemplary methods for circularizing linear RNA, including enzymatic ligation of a 5′ phosphate with a 3′-OH terminus (FIG. 2A), chemical ligation of a phosphate with OH-terminus (the 5′ or the 3′ end can be phosphorylated) (FIG. 2B); chemical ligation of a 3′ thiophosphate with a tosylated 5′ end (FIG. 2C); chemical ligation of a 3′-thiophosphate with a iodinated 5′-end (FIG. 2D); chemical ligation of a 3′-aldehyde with a 50 oxoamine (oxime circularization) (FIG. 2E); chemical ligation of a 5′- or 3′-azide with a 3′- or 5′-alkyne (Click circularization) (FIG. 2F); circularization by metal chelation (M=Zn²⁺ or Fe²⁺, (=terpyridine)) (FIG. 2G).

FIG. 3 is a schematic showing an illustrative method for circularizing linear RNA. In the intron-exon construct shown, a group I catalytic intron of the T4 phage Td gene is bisected in such a way to preserve structural elements critical for ribozyme folding. Exon fragment 2 (E2) is then ligated upstream of exon fragment 1 (E1), and a coding region roughly 1.1 kb in length is inserted between the exon-exon junction. During splicing, the 3′ hydroxyl group of a guanosine nucleotide engages in a transesterification reaction at the 5′ splice site, resulting in circularization of the intervening region and excision of the 3′ intron.

FIG. 4 provides a scheme of a timeline and process for the reprogramming of CD34+ cells in suspension.

FIG. 5 provides representative images of iPSC colony formation during CD34+ cell reprogramming.

FIG. 6A-FIG. 6C provide immunofluorescence images of Tra-1-81+ and Oct4+ cells after reprogramming with various amounts of the C14 reprogramming cocktail. FIG. 6A provides images of iPSCs made by treatment of CD34+ cells in suspension with 5.2 pg of the C14 cocktail transfected on day 0 and day 2. FIG. 6B provides images of iPSCs made by treatment of CD34+ cells in suspension with 13 pg of the C14 cocktail transfected on day 0 and day 2. FIG. 6C provides images of iPSCs made by treatment of CD34+ cells in suspension with 5.2 pg of the C14 cocktail transfected on day 0, day 1, day 2, and day 3.

FIG. 7 provides representative phase contrast images of circRNA-reprogrammed iPSC clones at passage 1.

FIG. 8 shows the Tra-1-81+ and Oct4+ staining results for a 1-step simultaneous editing and reprogramming protocol.

FIG. 9 provides representative phase contrast images of iPSC clones derived from the 1-step simultaneous reprogramming and editing protocol.

FIG. 10 shows Tra-1-81+ and Oct4+ staining results for a 2-step editing and reprogramming protocol.

FIG. 11 provides a scheme of a timeline and experimental design for reprogramming of CD34+ cells in suspension.

FIG. 12A-FIG. 12C provide phase contrast images showing the morphological progression of CD34+ cell reprogramming using C14 (FIG. 12A), C6 (FIG. 12B), and C11 (FIG. 12C) circRNA cocktails.

FIG. 13A-FIG. 13B show results of CD34+ cell reprogramming. FIG. 13A provides a representative image of a plate of iPSC-like colonies with varying plating densities. FIG. 13B provides a comparison of the reprogramming efficiencies observed early (d8-d10) during reprogramming with different circRNA cocktails.

FIG. 14 provides a scheme of the experimental design for optimization of the electroporation protocol for CD34+ cells.

FIG. 15 shows the kinetics of nGFP protein expression after electroporation as determined by IncuCyte imaging.

FIG. 16 illustrates flow cytometry analysis of nGFP protein levels one day after the first transfection by electroporation.

FIG. 17 provides the results of nGFP protein expression in fibroblasts (HDFs) after RNA transfection with RNAiMAX (50 ng/24-well) as analyzed by IncuCyte.

FIG. 18 provides the results of nGFP protein expression in CD34+ cells after RNA transfection of suspension cultured cells using Neon® (250 ng/6-well) as analyzed by IncuCyte.

FIG. 19 provides a scheme of the experimental design for assessing a reduced number of reprogramming factors for reprogramming of CD34+ cells in suspension.

FIG. 20A-FIG. 20B show phase contrast images of iPSC clones generated with reprogramming cocktails C6, C5, C4, and C3. FIG. 20A shows phase contrast images of iPSC clones illustrating the morphological progression over time with reprogramming cocktails C6, C5, C4, and C3. FIG. 20B shows a comparison of the iPSC morphology on Day 14 with the C6, C5, C4, and C3 reprogramming cocktails.

FIG. 21 shows phase contrast images of iPSC clones reprogrammed with two different concentrations of the C6 cocktail.

FIG. 22 shows phase contrast images demonstrating a lack of iPSC clone formation after reprogramming with two transfections of the S6 or L6 reprogramming cocktails.

FIG. 23 shows phase contrast images of iPSC clones reprogrammed with the C6 reprogramming cocktails compared to a lack of iPSC clone formation with the S6 or L6 reprogramming cocktails on Day 14.

FIG. 24A-FIG. 24B show reprogramming efficiencies achieved with different reprogramming cocktails and concentrations thereof. FIG. 24A provides the reprogramming efficiencies for CD34+ cells observed with various reprogramming cocktails. FIG. 24B provides the reprogramming efficiencies for CD34+ cells observed with various concentrations of the C6 reprogramming cocktail.

FIG. 25 shows staining with pluripotency markers Tra-1-81 (green) and OCT4 (red) after transfection with various concentrations of the C6 cocktail.

FIG. 26 shows staining with pluripotency markers Tra-1-81 (green) and OCT4 (red) after transfection with C3, C4, or C5 cocktails.

FIG. 27 provides a schematic of the experimental design for characterizing circRNA reprogrammed iPSCs in long-term in vitro culturing conditions up to 20 cell passages.

FIG. 28A-FIG. 28B show phase contrast images of iPSC clones generated with reprogramming cocktails C14, C6, C4, and C3. FIG. 28A shows phase contrast images illustrating the morphology of iPSC clones derived from mixed donor CD34+ cells at cell passages 10, 15, and 20 with reprogramming cocktails C14, C6, and C4.

FIG. 28B shows phase contrast images illustrating the morphology of iPSC clones derived from single donor CD34+ cells at cell passages 10, 15, and 20 with reprogramming cocktails C6, C4, and C3.

FIG. 29A-FIG. 29B provides the results of the percent viability of iPSCs derived from cord blood CD34+ cells with reprogramming cocktails C14, C6, C4 and C3 as analyzed by Incucyte. FIG. 29A provides the results of the percent viability of iPSCs derived from mixed donor cord blood CD34+ cells with reprogramming cocktails C14, C6, and C4 at each of cell passages 4-20, as analyzed by Incucyte. FIG. 29B provides the results of the percent viability of iPSCs derived from single donor cord blood CD34+ cells with reprogramming cocktails C6, C4, and C3 at each of cell passages 4-18, as analyzed by Incucyte.

FIG. 30A-FIG. 30B provides the results of the population doubling time of iPSCs derived from cord blood CD34+ cells with reprogramming cocktails C14, C6, C4 and C3 as analyzed by Incucyte. FIG. 30A provides the results of the population doubling time of iPSCs derived from mixed donor cord blood CD34+ cells with reprogramming cocktails C14, C6, and C4 at each of cell passages 5-20, as analyzed by Incucyte. FIG. 30B provides the results of the population doubling time of iPSCs derived from single donor cord blood CD34+ cells with reprogramming cocktails C6, C4, and C3 at each of cell passages 4-20, as analyzed by Incucyte.

FIG. 31A-FIG. 31B provides the results of the cumulative population doublings of iPSCs derived from cord blood CD34+ cells with reprogramming cocktails C14, C6, C4 and C3 as analyzed by Incucyte. FIG. 31A provides the results of the cumulative population doublings of iPSCs derived from mixed donor cord blood CD34+ cells with reprogramming cocktails C14, C6, and C4 at starting at cell passages 5-8, through cell passage 20, as analyzed by Incucyte. FIG. 31B provides the results of the cumulative population doublings of iPSCs derived from single donor cord blood CD34+ cells with reprogramming cocktails C6, C4, and C3 at each of cell passages 4-20, as analyzed by Incucyte.

FIG. 32 shows illustrative fluorescent images of the staining of the three primary germ cell layers with DAPI for counterstaining, markers SOX17 and FOXA2 for endoderm, T (brachyury) and FOXA2 for mesoderm and PAX6 for ectoderm, after reprogramming with C14, C6, C4 or C3 cocktail.

### DETAILED DESCRIPTION

### Overview

The present disclosure provides compositions and methods for reprogramming of cells comprising contacting the cells with circular RNA polynucleotides (circRNA) encoding one or more reprogramming factors. In various embodiments, the methods disclosed herein provide surprisingly efficient reprogramming, which enables reprogramming to occur with lower numbers of transfections and with the use of minimal reprogramming factors. Importantly, these features of the presently disclosed methods enable the reprogramming of hard to transfect cells including blood cells (e.g., CD34+ cells, T cells, B cells, NK cells, NKT cells, peripheral blood mononucleocytes, cord blood mononucleocytes) directly in suspension culture. This is a major advance in reprogramming technology, as blood cells are known to be refractory to transfection. See Warren et al., Molecular Therapy. 2019; 27(4): 729-734. As is shown herein, the harsh methods utilized in the prior art to transfect more robust cell types like fibroblasts are not suitable for use in reprogramming CD34+ cells. Furthermore, the methods disclosed herein do not work for linear RNA species-rather, we surprisingly demonstrate that circRNA is uniquely able to reprogram these sensitive cells. The present disclosure further provides methods and compositions for the simultaneous reprogramming and editing of cells comprising contacting the cells with (i) circRNAs encoding one or more reprogramming factors; (ii) an enzyme capable of editing the DNA or RNA of the cell; and (iii) optionally a guide RNA polynucleotide. Such simultaneous reprogramming and editing may be used to reprogram and edit blood-derived cells (e.g., CD34+ cells) directly in suspension culture using circular RNAs.

The reprogramming methods utilizing circRNA polynucleotides described herein provide advantages over reprogramming methods currently known in the art. As demonstrated herein, use of circRNAs encoding reprogramming factors unexpectedly reduces the total number of exogenous factors required to reprogram a cell. For example, the methods described herein achieve cell reprogramming by contacting the cell with circRNAs encoding the reprogramming factors Oct3/4, Sox2, Klf4, cMyc, Nanog, and Lin28, without the need for additional ancillary factors such as viral proteins including E3, K3, and B18R (Kogut et al., Nat Commun 9, 745 (2018). https://doi.org/10.1038/s41467-018-03190-3; Poleganov et al., Hum Gene Ther. 2015 November; 26(11): 751-66. doi: 10.1089/hum.2015.045) and/or exogenous miRNAs including miR302 (See Ying et al. Methods Mol Biol. 2018; 1733:283-304. doi: 10.1007/978-1-4939-7601-0_23) and miR367 (See Anokye-Danso et al. Cell Stem Cell. 2011; 8:376-388; Kogut et al., Nat Commun 9, 745 (2018)). The reprogramming methods disclosed herein, which do not require these ancillary factors, not only decrease the cost of manufacturing iPSCs by reducing the number of reagents needed, but also improve the safety of the iPSCs generated by avoiding potentially undesirable effects of introducing these ancillary factors (See e.g., Ventura and Jacks, MicroRNAs and cancer: short RNAs go a long way. Cell 2009; 586-91 describing the expression of certain microRNAs and their involvement in tumorigenesis; and Yu et al. Human microRNA clusters: genomic organization and expression profile in leukemia cell lines. Biochem Biophys Res Commun 2006; 349:59-68 describing the over-expression of the miR302/367 cluster in leukemic cell lines).

Furthermore, the present disclosure describes successful reprogramming of CD34+ cells in suspension using a reduced number of reprogramming factors than has been previously described. For example, the present disclosure demonstrates successful reprogramming with Oct3/4, Sox2, and Klf4 (without Nanog, Lin28A or LIN28B, and/or c-Myc), Oct3/4, Sox2, Klf4, and cMyc (without Nanog or Lin28A or Lin28B), and Oct3/4, Sox2, Klf4, Nanog, and Lin28A or LIN28B (without c-Myc). In addition to reducing the number of ancillary factors required, the present disclosure describes reduction in the number of transfections needed to successfully reprogram blood cells, including CD34+ cells, in suspension. Each of these improvements enhances the feasibility and success rate of the manufacturing process for reprogramming and importantly reduces stress on the starting cell population.

Furthermore, the compositions and methods described herein enable the “footprint-free” reprogramming of cells in suspension without requiring the use of viruses or integrating plasmids. Thus, the compositions and methods described herein enable easier manufacture of safe iPSC technologies than are available in the art. Previously described reprogramming methods utilize viral vectors or DNA plasmids to deliver the necessary transgenes to blood cells. These viruses either persist in the cell or integrate into the genome, and thus their clinical use is limited due to safety concerns. DNA plasmids can also integrate into the genome, resulting in significant numbers of manufacturing failures. Delivering RNA opens the possibility of “footprint free” reprogramming of cells, but previously described methods utilizing RNA have been unsuccessful in suspension cells. In particular, blood cells tend to be refractory to transfection with cationic reagents, which presents a major hurdle to their reprogramming with RNA polynucleotides, wherein cultures typically need to be transfected multiple times in order to sustain expression of RNA-encoded transgenes (Chong, Yeap and Ho, Transfection types, methods and strategies: a technical review (2021) PeerJ 9: e11165). Electroporation can achieve efficient mRNA delivery into blood cells, but the harshness of this procedure raises similar difficulties when repeat dosing of the RNA polynucleotides is necessary.

While mRNA reprogramming of endothelial progenitor cells that can be differentiated from peripheral blood CD34+ cells has recently been reported, this requires a 2-4 week differentiation process and reprogramming in an adherent cell culture. Therefore, there is still an unmet demand for an RNA-based reprogramming protocol that works with true blood lineages.

The present disclosure exemplifies reprograming of blood cells (e.g., CD34+ cells) in suspension. Reprogramming in suspension cultures provides many advantages over the adherent culture methods known in the art. For example, cells that can be grown in suspension are easy to source. There are numerous blood banks that are accessible for specific haplotypes, blood cell types, age etc., including those cord blood banks. Further, the methods described herein enable reprogramming of cells obtained directly from patient blood draws, rather than relying on cells obtained from biopsy samples that need to be grown in an adherent culture. As such, cells can likely be sourced from a wider range of donors, enabling the selection of a large spectrum of healthy donors potentially minimizing the use of starting cell types with pre-existing genomic abnormalities (e.g. skin fibroblasts). Furthermore, fibroblasts from older adults have been shown to be harder to reprogram (Narayan et al. OCT4 and SOX2 Work as Transcriptional Activators in Reprogramming Human Fibroblasts. Cell Rep. 2017; 20(7): 1585-1596. doi: 10.1016/j.celrep.2017.07.071); however, no such correlation with age has been found with reprogramming of blood cells (Hokayem et al. Blood Derived Induced Pluripotent Stem Cells (iPSCs): Benefits, Challenges and the Road Ahead. J Alzheimers Dis Parkinsonism. 2016 October; 6(5): 275. doi: 10.4172/2161-0460.1000275. Epub 2016 Oct. 25) Obtaining source material directly from blood draws also reduces time, reagent costs, invasiveness to patients, and reduces the degree of manipulation that the starting material must undergo in order to successfully obtain reprogrammed iPSCs. For example, many adherent cell types (e.g. fibroblasts, keratinocytes, endothelial progenitors) have to be characterized and expanded several passages in vitro before reprogramming.

Furthermore, the methods of the present disclosure enable long-term propagation of the circRNA-reprogrammed iPSCs (see Examples 7 and 8). The iPSCs generated according to the methods of the present disclosure are stable in their morphology, remain viable, undergo cell division at a consistent rate, and maintain cell identity which closely resembles ESCs after multiple cell passages and under long term cell culturing conditions (see Example 7). These iPSCs also remain genetically and epigenetically stable (see Example 8).

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

Unless the context indicates otherwise, it is specifically intended that the various features described herein can be used in any combination. Moreover, in some embodiments, any feature or combination of features set forth herein can be excluded or omitted. To illustrate further, if, for example, the specification indicates that a particular amino acid can be A, G, I, L and/or V, this language also indicates that the amino acid can be any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in some embodiments the amino acid is not A, G or I; is not A; is not G or V; etc., as if each such possible disclaimer is expressly set forth herein.

All publications, patent applications, patents, GenBank or other accession numbers and other references mentioned herein are incorporated by reference in their entirety for all purposes.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

The following terms are used in the description herein and the appended claims.

The singular forms “a,” “an” and “the” are intended to include the plural forms as well, unless the context clearly indicates otherwise.

The term “about” as used herein when referring to a measurable value such as an amount of the length of a polynucleotide or polypeptide, dose, time, temperature, and the like, is meant to encompass variations of ±20%, ±10%, ±5%, ±1%, ±0.5%, or even ±0.1% of the specified amount.

As used herein, “and/or” refers to and encompasses any and all possible combinations of one or more of the associated listed items, as well as the lack of combinations when interpreted in the alternative (“or”).

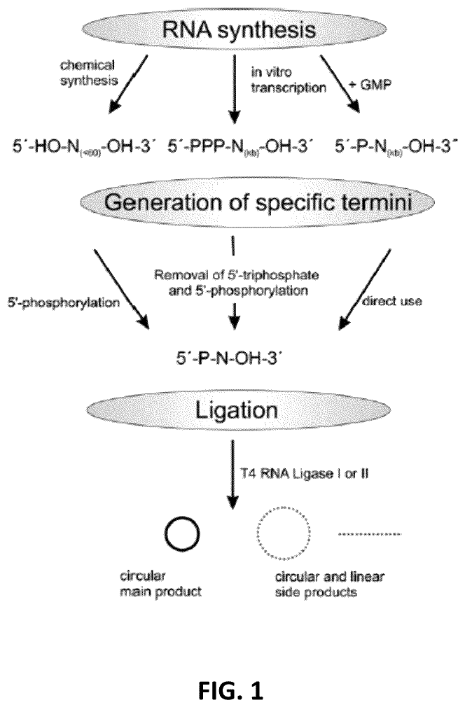

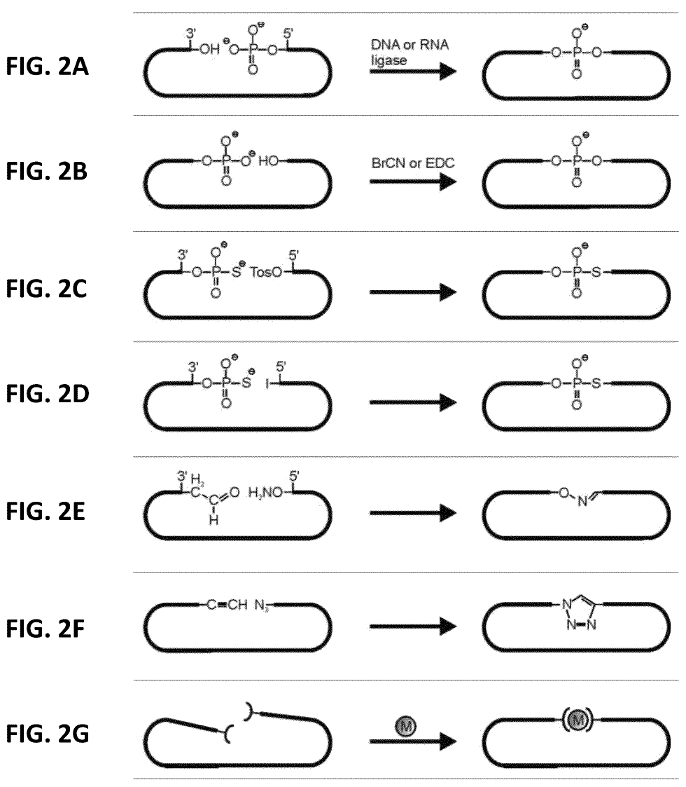

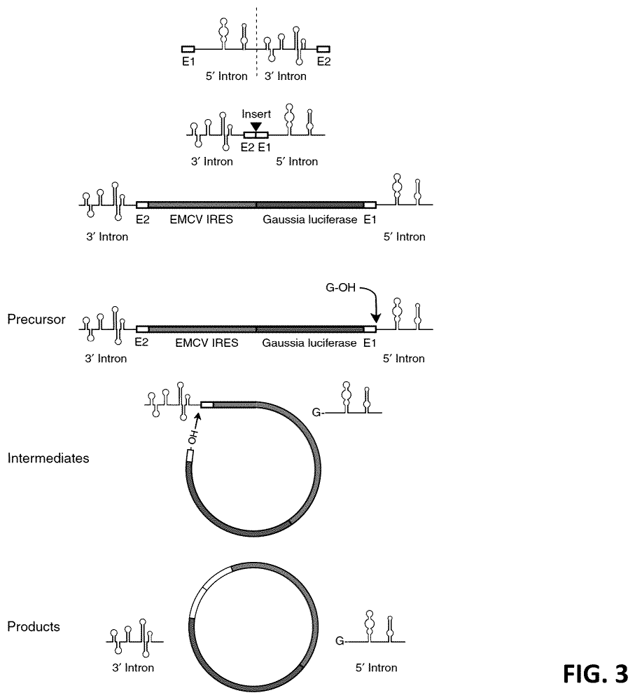

As used herein, “circular RNA” or “circRNA” refers to a type of single-stranded RNA which, unlike the better known linear RNA, forms a covalently closed continuous loop. Herein, any protein name preceded by “circ” refers to a circular RNA encoding that gene. RNAs may be circularized in a cell, by the cellular splicing machinery. For example, circular RNAs may be generated when the pre-mRNA splicing machinery “backsplices” to join a splice donor to an upstream splice acceptor, thereby producing a circular RNA that has covalently linked ends. Alternatively, circular RNAs may be generated in vitro, for example by circularization of a linear RNA produced by in vitro transcription (IVT). There are three general strategies for in vitro RNA circularization: chemical methods using cyanogen bromide or a similar condensing agent, enzymatic methods using RNA or DNA ligases (e.g., T4 RNA ligase I or II), and ribozymatic methods using self-splicing introns. A ribozymatic method utilizing a permuted group I catalytic intron is applicable for long RNA circularization and requires only the addition of GTP and Mg²⁺ as cofactors. This permuted intron-exon (PIE) splicing strategy consists of fused partial exons flanked by half-intron sequences. In vitro, these constructs undergo the double transesterification reactions characteristic of group I catalytic introns, but because the exons are already fused they are excised as covalently 5′ to 3′ linked circles (See FIG. 3). An illustrative protocol for circularizing linear RNA is provided in FIG. 1 and a list of illustrative linear RNA circularization strategies is provided in FIG. 2A-2G.

The terms “linear RNA” and “linear mRNA” are used interchangeably herein, as will be evident to a person of ordinary skill in the art based on context.

As used herein, “pluripotent” refers to a cell with the capacity, under different conditions, to differentiate to more than one differentiated cell type, and to differentiate to cell types characteristic of all three germ cell layers. In some embodiments, pluripotency may be evidenced by the expression of one or more pluripotent stem cell markers.

As used herein, the terms “induced pluripotent stem cells” and “iPSCs” refer to pluripotent cells that are generated from various differentiated (i.e., multipotent or non-pluripotent) somatic cells. iPSCs are substantially genetically identical to their respective differentiated somatic cell of origin and display characteristics similar to higher potency cells, such as embryonic stem (ES) cells, including the capacity to indefinitely self-renew in culture and the capacity to differentiate into other cell types. In some embodiments, iPSCs exhibit morphological (i.e., round shape, large nucleoli and scant cytoplasm) and growth properties (i.e., doubling time) akin to ES cells. In some embodiments, iPSCs express pluripotent cell-specific markers (e.g., Oct-4, SSEA-3, SSEA-4, Tra-1-60, Tra-1-81, but not SSEA-1).

As used herein, a “differentiated cell” or “somatic cell” is any cell that is not, in its native form, pluripotent as that term is defined herein. The term “somatic cell” also encompasses progenitor cells that are multipotent (e.g., can produce more than one cell type) but not pluripotent (e.g., can produce cells from all three germ layers).

The term “reprogramming” as used herein refers to a process of altering the differentiation state of a cell, such as a somatic cell, multipotent cell or progenitor cell. In some embodiments, reprogramming a cell may comprise converting a cell from a first cell type to a second cell type. In some embodiments, reprogramming may comprise altering the phenotype of a differentiated cell to a pluripotent phenotype. In some embodiments, reprogramming may refer to a process of “induced differentiation” or “transcription factor-directed differentiation” wherein an iPSC is converted into a differentiated cell.

As used herein, the term “reprogramming factor” refers to any factor or combination of factors that promotes the reprogramming of a cell. A reprogramming factor may be, for example, a transcription factor. Illustrative reprogramming factors for producing iPSCs from differentiated cells include Oct3/4, Klf4, Sox2, Nanog, Lin28 (e.g., Lin28A or Lin28B), c-Myc, and L-Myc. Illustrative reprogramming factors and combinations thereof for producing reprogrammed cells are provided in Tables 1 and 3.

As used herein, “transdifferentiation” refers to a type of cellular reprogramming wherein one somatic cell type is directly converted into a second somatic cell type. In some embodiments, transdifferentiation may refer to “direct reprogramming” or “direct cell-fate conversion” wherein a somatic cell of a first cell type is converted into a somatic cell of a second cell type without going through an intermediate pluripotent state or progenitor cell type.

As used herein, “Internal ribosome entry site” or “IRES” is an RNA element that allows for initiation of translation in a cap-independent manner. An IRES may be, for example, a viral IRES or a mammalian IRES (e.g., a human IRES).

A “nucleotide triphosphate” or “NTP” is a molecule comprising a nitrogenous base bound to a 5-carbon sugar (either ribose or deoxyribose), with three phosphate groups bound to the sugar.

As used herein, a “modified NTP” is a NTP that has been chemically modified to confer favorable properties to a nucleic acid comprising the NTP. Such favorable properties may include, for example, reduced immunogenicity, increased stability, chemical functionality, or modified binding affinity.

The term “modified RNA” (e.g., “modified linear RNA” or “modified circular RNA”) is used to describe an RNA molecule which comprises one or more modified NTPs.

The term “vector” refers to a carrier for a nucleic acid (i.e., a DNA or RNA molecule), which can be used to introduce the nucleic acid into a cell. An “expression vector” is a vector that comprises a sequence encoding a protein or an RNA (e.g., a circular RNA) and the necessary regulatory regions needed for expression of the sequence in a cell. In some embodiments, the sequence encoding a protein or an RNA is operably linked to another sequence in the vector. The term “operably linked” means that the regulatory sequences necessary for expression of the sequence encoding a protein or an RNA are placed in the nucleic acid molecule in the appropriate positions relative to the sequence to effect expression of the protein or RNA.

As used herein, the terms “lipid nanoparticle” and “LNP” describe lipid-based particles in the submicron range. LNPs can have the structural characteristics of liposomes and/or may have alternative non-bilayer types of structures. LNPs may be conjugated to nucleic acids (e.g., DNA or RNA molecules) and used to deliver the nucleic acid to cells.

The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polypeptide sequence or at least one nucleic acid difference (e.g., nucleic acid substitution, nucleic acid insertion, nucleic acid deletion) relative to a reference polynucleotide, e.g. a native polynucleotide or polypeptide sequence. For example, a variant may be a polynucleotide having a sequence identity of 50% or more, 60% or more, or 70% or more with a full length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g., an identity of 75% or 80% or more, such as 85%, 90%, or 95% or more, for example, 98% or 99% identity with the full length native polypeptide sequence. Variants may also include variant fragments of a reference, e.g., native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

Methods of determining sequence similarity or identity between two or more nucleic acid sequences or amino acid sequences are known in the art. For example, sequence similarity or identity may be determined using the local sequence identity algorithm of Smith & Waterman, Adv. Appl. Math. 2, 482 (1981), by the sequence identity alignment algorithm of Needleman & Wunsch J Mol. Biol. 48, 443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al. Nucl. Acid Res. 12, 387-395 (1984), or by inspection.

Another suitable algorithm is the BLAST algorithm, described in Altschul et al. J. Mol. Biol. 215, 403-410, (1990) and Karlin et al. Proc. Natl. Acad. Sci. USA 90, 5873-5787 (1993). A particularly useful BLAST program is the WU-BLAST-2 program which was obtained from Altschul et al. Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

### Circular RNAs

Provided herein are circular RNAs. In particular embodiments, the circular RNAs encode reprogramming factors that are (alone or in combination with other reprogramming factors) capable of reprogramming differentiated cells into iPSCs, capable of differentiating iPSCs into differentiated cells, and/or capable of differentiating one differentiated cell type into another differentiated cell type.

In some embodiments, a circular RNA comprises from about 200 nucleotides to about 5,000 nucleotides. In some embodiments, the circular RNA comprises from about 200 to about 1,000 nucleotides. In some embodiments, the circular RNA comprises from about 1,000 nucleotides to about 2,500 nucleotides. In some embodiments, the circular RNA comprises from about 2,500 nucleotides to about 5,000 nucleotides. In some embodiments, the circular RNA comprises more than about 5,000 nucleotides.

In some embodiments, a circular RNA comprises one or more open reading frames. In some embodiments, a circular RNA comprises one or more protein-coding sequences. In some embodiments, a circular RNA does not comprise an open reading frame, and/or a protein-coding sequence.

In embodiments wherein the circular RNA comprises more than one protein-coding nucleic acid sequence, each sequence may be separated by a sequence encoding a self-cleaving peptide, such as a 2A peptide. Illustrative 2A peptides include, but are not limited to, EGRGSLLTCGDVEENPGP (SEQ ID NO: 1), ATNFSLLKQAGDVEENPGP (SEQ ID NO: 2), QCTNYALLKLAGDVESNPGP (SEQ ID NO: 3), and VKQTLNFDLLKLAGDVESNPGP (SEQ ID NO: 4). In some embodiments, each protein-encoding nucleic acid sequence may be separated by an IRES.

Circular RNAs lack a 5′ 7-methylguanosine cap structure which is required for efficient translation of linear mRNAs. For a circular RNA to be translated, therefore, an alternative mechanism of recruiting the ribosome may be used. For example, an internal ribosome entry site (IRES) may be used, which directly binds initiation factors or the ribosome itself. Accordingly, in some embodiments, a circular RNA comprises an internal ribosome entry site (IRES). In some embodiments, the IRES engages a eukaryotic ribosome. In some embodiments, the IRES is operatively linked to a protein-encoding nucleic acid sequence.

Examples of IRES sequences include sequences derived from a wide variety of viruses, for example from leader sequences of picornavirus UTR's (such as the encephalomyocarditis virus (EMCV)), the polio leader sequence, the hepatitis A virus leader, the hepatitis C virus IRES, human rhinovirus type 2 IRES, an IRES element from the foot and mouth disease virus, a giardiavirus IRES, and the like. A variety of nonviral IRES sequences may also be used, including, but not limited to IRES sequences from yeast, as well as the human angiotensin II type 1 receptor IRES, fibroblast growth factor IRESs, vascular endothelial growth factor IRES, and insulin-like growth factor 2 IRES. Additional IRES sequences suitable for use in the circular RNAs described herein include those described in the database available at http://iresite.org/.

In some embodiments, the circular RNA comprises intronic elements that flank the protein-encoding sequence. Intronic elements may be backspliced by cellular splicing machinery to yield a circular RNA that is covalently closed. Accordingly, in some embodiments, a circular RNA comprises a first intronic element located 5′ to the protein-encoding sequence, and a second intronic element located 3′ to the protein-encoding sequence.

In some embodiments, a circular RNA is generated by circularizing a linear RNA. In some embodiments, a linear RNA may be self-circularizing, for example if it comprises self-splicing introns. Because circular RNAs do not have 5′ or 3′ ends, they may be resistant to exonuclease-mediated degradation and may be more stable than most linear RNAs in cells.

In some embodiments, the intronic elements are selected from any known intronic element(s), in any combination and in any multiples and/or ratios. Examples of intronic elements include those described in the circBase circular RNA database (Glazar et al. RNA 20:1666-1670 (2014); and www.circbase.org) and in Rybak-Wolf et al. Mol. Cell 58(5): 870-885 (2015), each of which are incorporated by reference herein in their entirety. In some embodiments, the intronic element is a mammalian intron or a fragment thereof. In some embodiments, the intronic element is a non-mammalian intron (e.g., a self-splicing group I intron, a self-splicing group II intron, a spliceosomal intron, or a tRNA intron), or a fragment thereof.

In some embodiments, the circular RNA comprises one or more additional elements which improves the stability and/or translation of the protein-encoding sequence from the circular RNA. For example, in some embodiments, the circular RNA may comprise a Kozak sequence. One example of a Kozak consensus sequence is: RCC (AUG) G (SEQ ID NO: 5), with the start codon in parentheses, and the “R” at position −3 representing a purine (A or G). Another example of a Kozak consensus sequence is RXY (AUG) (SEQ ID NO: 6), where R is a purine (A or G), Y is either C or G, and X is any base.

In some embodiments, a circular RNA comprises a first intronic element, a protein-encoding sequence, and a second intronic element. In some embodiments, a circular RNA comprises an IRES and a protein-encoding sequence. In some embodiments, a circular RNA comprises a first intronic sequence, an IRES, a protein-encoding sequence, and a second intronic sequence.

In some embodiments, a circular RNA comprises a sequence encoding a reprogramming factor (e.g., a transcription factor). In some embodiments, a circular RNA comprises a first intronic element, a sequence encoding a reprogramming factor, and a second intronic element.

In some embodiments, a circular RNA comprises an IRES and a sequence encoding a reprogramming factor. In some embodiments, a circular RNA comprises a first intronic sequence, an IRES, a sequence encoding a reprogramming factor, and a second intronic sequence. In some embodiments, a circular RNA comprises an IRES and a sequence encoding a reprogramming factor. In some embodiments, a circular RNA comprises a first intronic element, an IRES, a sequence encoding a reprogramming factor, and a second intronic element. See also US 2020/0080106, which is incorporated herein by reference. Circular RNAs may also comprise modified bases and/or NTPs. In some embodiments, the circular RNAs comprise modified NTPs. In some embodiments, the circular RNAs are modified circular RNAs.

Modified bases include synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines s such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone.

In some embodiments, the circular RNAs comprise modified backbones. Examples of modified RNA backbones include those that comprise phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl-phosphoramidates, thionophosphoramidates, thionoalkylphosphonates, thionoalkyl-phosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

In some embodiments, the circular RNAs may be modified by chemically linking to the RNA one or more moieties or conjugates which enhance the activity, cellular distribution or cellular uptake. For example, a circular RNA may be conjugated to intercalators, reporter molecules, polyamines, polyamides, polyethylene glycols, polyethers, groups that enhance the pharmacodynamic properties of oligomers, or groups that enhance the pharmacokinetic properties of oligomers. In some embodiments, the circular RNAs may be conjugated to cholesterols, lipids, phospholipids, biotin, phenazine, folate, phenanthridine, anthraquinone, acridine, fluoresceins, rhodamines, coumarins, or dyes. Groups that enhance the pharmacodynamic properties include groups that improve RNA uptake, enhance oligomer resistance to degradation, and/or strengthen sequence-specific hybridization with RNA. Groups that enhance the pharmacokinetic properties include groups that improve oligomer uptake, distribution, metabolism or excretion. The circular RNAs may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. In some embodiments, a circular RNA is conjugated to a lipid nanoparticle (LNP).

In some embodiments, the circular RNA is part of a complex. In some embodiments, a complex comprises a circular RNA and a lipid nanoparticle (LNP). In some embodiments, the circular RNA and the LNP are conjugated. In some embodiments, the circular RNA and the LNP are covalently conjugated. In some embodiments, the circular RNA and the LNP are non-covalently conjugated. In some embodiments, the methods described herein comprise contacting a cell with one or more circular RNAs that have been complexed with an LNP.

The LNP may comprise, for example, one or more cationic lipids, non-cationic lipids, and/or PEG-modified lipids. In some embodiments, the LNP may comprise at least one of the following cationic lipids: C12-200, DLin-KC2-DMA, DODAP, HGT4003, ICE, HGT5000, or HGT5001. In some embodiments, the LNP comprises cholesterol and/or a PEG-modified lipid. In some embodiments, the LNP comprises DMG-PEG2K. In some embodiments, the LNP comprises one of the following: C12-200, DOPE, cholesterol, DMG-PEG2K; DODAP, DOPE, cholesterol, DMG-PEG2K; HGT5000, DOPE, cholesterol, DMG-PEG2K, HGT5001, DOPE, or DMG-PEG2K. In some embodiments, the LNP comprises polyethyleneimine (PEI).

In some embodiments, the circular RNA is substantially non-immunogenic. In some embodiments, a circular RNA is considered non-immunogenic if it does not induce the expression or activity of one or more interferon-regulated genes (e.g., one or more genes described at www.interferome.org). In some embodiments, the interferon-regulated genes are selected from IFN-alpha, IFN-beta, and/or TNF-alpha. Various modifications can be made to the circular RNA to reduce the immunogenicity thereof. For example, in some embodiments, the circular RNA may be modified to comprise one or more M-6-methyladenosine (m⁶A), 5-methyl-cytosine (5mC), or pseudouridine residues.

In some embodiments, the circular RNAs described herein are less immunogenic than linear RNA. For example, in some embodiments, a circular RNA does not substantially induce the expression and/or activity of one or more interferon-regulated genes. In some embodiments, a circular RNA induces the expression and/or activity of one or more interferon-regulated genes about 10%, about 20%, about 30%, about 40%, about 50%, about 60%, about 70%, about 80%, about 90%, or about 100% less than a linear RNA.

In some embodiments, the circular RNAs described herein have a longer cellular half-life than linear RNA. For example, a circular RNA may have a half-life that is about 10%, about 20%, about 30%, about 40%, about 50%, about 60%, about 70%, about 80%, about 90%, or about 100%, 2-fold, 3-fold, 4-fold, 5-fold, 10-fold or more, longer than that of a linear RNA. In some embodiments, a circular RNA may have a half-life that is about 4 hours, about 12 hours, about 18 hours, about 24 hours, about 2 days, about 3 days, about 4 days, about 5 days, about 10 days, or about 10 days longer than that of a linear RNA.

In some embodiments, the circular RNAs do not replicate in the cells. In some embodiments, the circular RNAs are risk-free for genome integration. As such, the circular RNAs described herein provide for “foot-print free” modifications of cells, wherein the genomic DNA of the cell remains unmodified by the circular RNA compositions.

Circular RNAs may be generated using any suitable method know in the art. In some embodiments, circular RNAs are generated using in vitro transcription (IVT), according to standard protocols and/or by using commercially-available kits (e.g., the MAXIscript® or MEGAscript® kits from ThermoFisher®). For example, an illustrative IVT protocol uses a purified linear DNA template (i.e., a DNA molecule encoding a circular RNA as described herein), ribonucleotide triphosphates, a buffer system that includes DTT and magnesium ions, and an appropriate phage RNA polymerase to produce a circular RNA. The DNA template contains a double-stranded promoter region where the phage polymerase binds and initiates RNA synthesis. Reaction conditions (e.g., the type of nucleotide salt, type and concentration of salt in the transcription buffer, enzyme concentration and pH) are optimized for the particular polymerase used and for the entire set of components, in order to achieve optimal yields. Large-scale IVT reactions can produce up to 120-180 μg RNA per microgram template in a 20 μl reaction. In some embodiments, circular RNAs may be generated using RNA synthesis, according to standard protocols.

Various methods for circularizing RNAs are known in the art. For example, an illustrative protocol for circularizing linear RNA is provided in FIG. 1 and a list of illustrative linear RNA circularization strategies is provided in FIG. 2A-2G. In some embodiments, an RNA is self-circularizing, for example, if it contains self-splicing introns.

Also provided herein are nucleic acids (i.e., DNA molecules) encoding the circular RNAs described herein, and vectors comprising the same.

### Circular RNAs Encoding Reprogramming Factors

In some embodiments, a circular RNA encodes a reprogramming factor. In some embodiments, the reprogramming factor is a human or humanized reprogramming factor. In some embodiments, the reprogramming factor is a transcription factor.

In some embodiments, the reprogramming factor may be, for example, any one of the reprogramming factors listed in in Table 1. In some embodiments, the reprogramming factor is a fragment or variant of any one of the reprogramming factors listed in Table 1. In some embodiments, the reprogramming factor has at least 90%, at least 95%, or at least 99% sequence identity to any one of the reprogramming factors listed in Table 1.

TABLE 1
Exemplary Reprogramming Factors

| | NCBI REFSEQ mRNA |
|---|---|
| Human Gene Symbol | Accession Number(s) |
| POU2F1 | NM_002697 |
| POU2F2 | NM_002698 |
| POU2F3 | NM_014352 |
| POU3F1 | NM_002699 |
| POU3F2 | NM_005604 |
| POU3F3 | NM_006236 |
| POU3F4 | NM_000307 |
| POU5F1 (encodes OCT4) | NM_002701, NM_203289 |
| POU5F2 | NM_153216 |
| POU6F1 | NM_002702, NR_026893 |
| POU6F2 | NM_007252 |
| SOX1 | NM_005986 |
| SOX2 | NM_003106 |
| SOX3 | NM_005634 |
| SOX4 | NM_003107 |
| SOX5 | NM_006940, NM_152989, |
| | NM_178010 |
| SOX6 | NM_017508, NM_033326, |
| | NM_001145811, |
| | NM_001145819 |
| SOX7 | NM_031439 |
| SOX8 | NM_014587 |
| SOX9 | NM_000346 |
| SOX10 | NM_006941 |
| SOX11 | NM_003108 |
| SOX12 | NM_006943 |
| SOX13 | NM_005686 |
| SOX14 | NM_004189 |
| SOX15 | NM_006942 |
| SOX17 | NM_022454 |
| SOX18 | NM_018419 |
| SOX21 | NM_007084 |
| SOX30 | NM_178424, NM_007017 |
| KLF1 | NM_006563 |
| KLF2 | NM_016270 |
| KLF3 | NM_016531 |
| KLF4 | NM_004235 |
| KLF5 | NM_001730 |
| KLF6 | NM_001300 |
| KLF7 | NM_003709 |
| KLF8 | NM_007250, NM_001159296 |
| KLF9 | NM_001206 |
| KLF10 | NM_005655, NM_001032282 |
| KLF11 | NM_003597 |
| KLF12 | NM_007249 |
| KLF13 | NM_015995 |
| KLF14 | NM_138693 |
| KLF15 | NM_014079 |
| KLF16 | NM_031918 |
| KLF17 | NM_173484 |
| POU5F1P1 | NR_002304 |
| MYC | NM_002467 |
| MYCL1 | NM_005376, NM_001033081, |
| | NM_001033082 |
| MYCN | NM_005378 |
| NANOG | NM_024865 |
| LIN28A | NM_024674 |
| LIN28B | NM_001004317 |
| THAP11 | NM_020457 |
| TERT | NM_198253, NM_198255 |
| MYOD1 | NM_002478 |
| ASCL1 | NM_004316 |
| SPI1 | NM_003120, NM_001080547 |
| CEBPA | NM_004364 |
| CEBPB | NM_005194 |
| NEUROG3 | NM_020999 |
| PDX1 | NM_000209 |
| MAFA | NM_201589 |
| ESRRB | NM_004452.2 |
| NKX3-1 | NM_006167 |
| GATA3 | NM_001002295 |

In some embodiments, the reprogramming factor is any one of the following reprogramming factors: Oct4, Sox2, Klf4, c-Myc, Lin28 (e.g., Lin28A or Lin28B), Nanog, SalI4, Utf1, p53, p21, p16^{lnk4a}, GLIS1, L-Myc, TGF-beta, MDM2, REM2, Cyclin D1, SV40 large T antigen, DOT1L, CX43, MBD3, SIRT6, TCL1a, RARy, SNAIL, Lrh-1, or RCOR2, or a combination thereof. In some embodiments, the reprogramming factor is a fragment or variant of any one of the following reprogramming factors: Oct4, Sox2, Klf4, c-Myc, Lin28 (e.g., Lin28A or Lin28B), Nanog, SalI4, Utf1, p53, p21, p16^{lnk4a}, GLIS1, L-Myc, TGF-beta, MDM2, REM2, Cyclin D1, SV40 large T antigen, DOT1L, CX43, MBD3, SIRT6, TCL1a, RARy, SNAIL, Lrh-1, or RCOR2, or a combination thereof.

In some embodiments, the reprogramming factor is any one of Oct3/4, Klf4, Sox2, Nanog, Lin28 (e.g., Lin28A or Lin28B), c-Myc, and/or L-Myc, or a fragment or variant thereof. In some embodiments, the reprogramming factor is Oct3/4, Klf4, Sox2, Nanog, Lin28 (e.g., Lin28A or Lin28B), and/or c-Myc, or a fragment or variant thereof. In some embodiments, the reprogramming factor is Oct3/4, Klf4, and/or Sox2 or a fragment or variant thereof. In some embodiments, the reprogramming factor is Oct3/4, Klf4, Sox2, and/or c-Myc, or a fragment or variant thereof. In some embodiments, the reprogramming factor is Oct3/4, Klf4, Sox2, Nanog, and/or Lin28 (e.g., Lin28A or Lin28B), or a fragment or variant thereof. In some embodiments, the reprogramming factor is a human or a humanized reprogramming factor.

In some embodiments, a circular RNA encodes the reprogramming factor Oct3/4. In some embodiments, the encoded Oct3/4 has an amino acid sequence of SEQ ID NO: 7, or a sequence at least 90% or at least 95%, 96%, 97%, 98%, or 99% identical thereto. In some embodiments, the circular RNA encodes the reprogramming factor Oct3/4 and comprises or consists of the nucleic acid sequence of SEQ ID NO: 8. In some embodiments, the circular RNA encodes the reprogramming factor Oct3/4 and comprises a nucleic acid sequence that is at least 90% or at least 95%, 96%, 97%, 98%, or 99% identical to SEQ ID NO: 8.

In some embodiments, a circular RNA encodes the reprogramming factor Klf4. In some embodiments, the encoded Klf4 has the amino acid sequence of SEQ ID NO: 9 or 10, or a sequence at least 90% or at least 95%, 96%, 97%, 98%, or 99% identical thereto. In some embodiments, the circular RNA encodes the reprogramming factor Klf4 and comprises or consists of the nucleic acid sequence of SEQ ID NO: 11. In some embodiments, the circular RNA encodes the reprogramming factor Klf4 and comprises a nucleic acid sequence that is at least 90% or at least 95%, 96%, 97%, 98%, or 99% identical to SEQ ID NO: 11.

In some embodiments, a circular RNA encodes the reprogramming factor Sox2. In some embodiments, the Sox2 has the amino acid sequence of SEQ ID NO: 12, or a sequence at least 90% or at least 95%, 96%, 97%, 98%, or 99% identical thereto. In some embodiments, the circular RNA encodes the reprogramming factor Sox2 and comprises or consists of the nucleic acid sequence of SEQ ID NO: 13. In some embodiments, the circular RNA encodes the reprogramming factor Sox2 and comprises a nucleic acid sequence that is at least 90% or at least 95%, 96%, 97%, 98%, or 99% identical to SEQ ID NO: 13.

In some embodiments, a circular RNA encodes the reprogramming factor Nanog. In some embodiments, the Nanog has the amino acid sequence of SEQ ID NO: 14 or 15, or a sequence at least 90% or at least 95%, 96%, 97%, 98%, or 99% identical thereto. In some embodiments, the circular RNA encodes the reprogramming factor Nanog and comprises or consists of the nucleic acid sequence of SEQ ID NO: 16. In some embodiments, the circular RNA encodes the reprogramming factor Nanog and comprises a nucleic acid sequence that is at least 90% or at least 95%, 96%, 97%, 98%, or 99% identical to SEQ ID NO: 16.

In some embodiments, a circular RNA encodes the reprogramming factor Lin28A. In some embodiments, the Lin28A has the amino acid sequence of SEQ ID NO: 17, or a sequence at least 90% or at least 95%, 96%, 97%, 98%, or 99% identical thereto. In some embodiments, the circular RNA encodes the reprogramming factor Lin28A and comprises or consists of the nucleic acid sequence of SEQ ID NO: 18. In some embodiments, the circular RNA encodes the reprogramming factor Lin28A and comprises a nucleic acid sequence that is at least 90% or at least 95%, 96%, 97%, 98%, or 99% identical to SEQ ID NO: 18.

In some embodiments, a circular RNA encodes the reprogramming factor c-Myc. In some embodiments, the c-Myc has the amino acid sequence of SEQ ID NO: 19 or 20, or a sequence at least 90% or at least 95%, 96%, 97%, 98%, or 99% identical thereto. In some embodiments, the circular RNA encodes the reprogramming factor c-Myc and comprises or consists of the nucleic acid sequence of SEQ ID NO: 21. In some embodiments, the circular RNA encodes the reprogramming factor c-Myc and comprises a nucleic acid sequence that is at least 90% or at least 95%, 96%, 97%, 98%, or 99% identical to SEQ ID NO: 21.

In some embodiments, a circular RNA encodes the reprogramming factor L-Myc. In some embodiments, the L-Myc has the amino acid sequence of any one of SEQ ID NO: 22-24, or a sequence at least 90% or at least 95%, 96%, 97%, 98%, or 99% identical thereto. In some embodiments, the circular RNA encodes the reprogramming factor L-Myc and comprises or consists of the nucleic acid sequence of SEQ ID NO: 25. In some embodiments, the circular RNA encodes the reprogramming factor L-Myc and comprises a nucleic acid sequence that is at least 90% or at least 95%, 96%, 97%, 98%, or 99% identical to SEQ ID NO: 25.

In some embodiments, a circular RNA comprises two or more protein-encoding nucleic acid sequences. For example, the circular RNA may comprise three, four, five, or six protein-encoding sequences. In some embodiments, at least one of the protein-encoding sequences encodes a reprogramming factor (e.g., a transcription factor). In some embodiments, the circular RNA comprises two, three, four, five, or six protein-encoding sequences encoding a combination of reprogramming factors according to any one of combinations 1-106 in Table 3.

In some embodiments, a circular RNA comprises two or more protein-encoding sequences, wherein at least one of the protein-encoding sequences encodes a reprogramming factor. In some embodiments, a circular RNA comprises two or more protein-encoding sequences, wherein at least one of the protein-encoding sequences encodes one or more of Oct3/4, Klf4, Sox2, Nanog, Lin28 (e.g., Lin28A or Lin28B), c-Myc, or L-Myc, or fragments or variants thereof. In some embodiments, a circular RNA comprises two or more protein-encoding sequences, wherein each of the protein-encoding sequences encodes a reprogramming factor selected from Oct3/4, Klf4, Sox2, Nanog, Lin28 (e.g., Lin28A or Lin28B), c-Myc, and L-Myc, or fragments or variants thereof.

In some embodiments, a circular RNA comprises a sequence encoding Oct3/4, Klf4, Sox2, Nanog, Lin28 (e.g., Lin28A or Lin28B), c-Myc, or L-Myc. In some embodiments, a circular RNA comprises a first intronic element, a sequence encoding Oct3/4, Klf4, Sox2, Nanog, Lin28 (e.g., Lin28A or Lin28B), c-Myc, or L-Myc, and a second intronic element.

In some embodiments, a circular RNA comprises an IRES and a sequence encoding Oct3/4, Klf4, Sox2, Nanog, Lin28 (e.g., Lin28A or Lin28B), c-Myc, or L-Myc. In some embodiments, a circular RNA comprises a first intronic element, an IRES, a sequence encoding Oct3/4, Klf4, Sox2, Nanog, Lin28 (e.g., Lin28A or Lin28B), c-Myc, or L-Myc, and a second intronic element.

### RNA Polynucleotides Encoding Ancillary Factors

In some embodiments, the present disclosure provides RNA polynucleotides (e.g., linear RNA polynucleotides or circular RNAs) encoding an exogenous factor that aids in cellular reprogramming. In some embodiments, the exogenous factor is an RNA, such as a micro RNA (miRNA) or a long non-coding RNA (e.g., LINCRNA-ROR). miRNAs such as the miRNA302(a-d) cluster and miR367 have been shown to improve the efficiency of reprogramming when used in conjunction with reprogramming factors (See U.S. Pat. Nos. 8,791,248; 8,852,940; Poleganov et al., Human Gene Therapy. November 2015.751-766). For example, the miRNA may be any one of the miRNA302 family (e.g., miR302d, miR302a, miR302c and miR302b), miR367, miR766, miR200c, miR369, miR372, Let7, miR19a/b) or a fragment or variant thereof.

In some embodiments, the ancillary factor is a circular RNA that is enriched in human stem cells (e.g., human ESCs), such as circBIRC6, circCORO1C, or circMAN1A2. circBIRC6, circCORO1C and circMAN1A2. These circular RNAs are thought to act as a “miR sponge”. Thus, they may have a regulatory role in promoting pluripotency by counteracting certain miRNAs (e.g. miR34a and/or miR145) that are known to suppress expression of the pluripotency-associated transcription factors NANOG, SOX2 and OCT4 (Yu et al. Nat Commun 8, 1149 (2017)).

In some embodiments, the ancillary factor is one or more viral proteins that inhibit the innate immune response. The viral proteins may be, for example, inhibitors of RIG-1 (retinoic acid-inducible gene I) or PKR (protein kinase R) pathways. Exemplary viral proteins are provided below in Table 2. Exemplary viral proteins suitable for use in the methods described herein include, but are not limited to, B18R, E3, or K3 from vaccinia virus. An exemplary B18R sequence is provided as SEQ ID NO: 26. In some embodiments, the B18R protein has a sequence that is at least 90%, or at least 95% identical to SEQ ID NO: 26. An exemplary E3 sequence is provided as SEQ ID NO: 27. In some embodiments, the E3 protein has a sequence that is at least 90%, or at least 95% identical to SEQ ID NO: 27. An exemplary K3 sequence is provided as SEQ ID NO: 28. In some embodiments, the K3 protein has a sequence that is at least 90%, or at least 95% identical to SEQ ID NO: 28.

TABLE 2
Viral proteins for suppressing an innate immune response

| Protein | Virus |
|---|---|
| Gamma 34.5 | Herpes simplex virus (HSV) |
| VP35 | Ebola virus |
| Influenza NS1 | Influenza virus |
| pTRS1/pIRS1 | Human cytomegalovirus (CMV) |
| m142/m143 | Murine CMV |
| NSs | Rift Valley fever virus (RVFV) |
| E3L | Vaccinia virus |
| MC159L | Poxvirus |
| NSP3 | Rotavirus group C |
| NSP5 | Rotavirus group 1 |
| Us11 | Herpes simplex virus |
| SM | Epstein-Barr virus |
| OVIFNR | Parapoxvirus |
| Crm1 | Poxvirus |
| L(pro) | Foot-and-mouth disease virus |
| Us11 | Herpex simplex virus |
| E6 | Papilloma virus |
| Large T antigen | SV-40 |
| LANA2 | Herpes Virus |
| BILF1 | Epstein-Barr virus |
| NS5A | Hepatitis C virus |
| P58 | Influenza virus |
| SM | Epstein-Barr virus |
| vIRF-2 | Human herpes virus-8 |
| PK2 | Baculovirus |
| TAT | HIV-1 |
| K3L | Vaccinia virus, Iridoviridae |
| S-HDAg | Hepatitis D virus |
| E2 | Hepatitis C virus |
| C8L | Swinepox virus |

### Circular RNA Compositions

In some embodiments, the present disclosure provides compositions of circular RNAs encoding reprogramming factors. In some embodiments, the composition further comprises a buffer. The buffer may comprise, for example, 1-10 mM sodium citrate. In some embodiments the PH of the buffer is about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12. In some embodiments, the pH of the buffer is about 6.5.

In some embodiments, the composition comprises two or more circular RNAs, each encoding a reprogramming factor selected from Oct3/4, Klf4, Sox2, Nanog, Lin28 (e.g., Lin28A or Lin28B), c-Myc, and L-Myc. In some embodiments, the composition comprises two or more circular RNAs, each encoding a reprogramming factor selected from Oct3/4, Klf4, Sox2, Nanog, Lin28 (e.g., Lin28A or Lin28B), and c-Myc. In some embodiments, the composition comprises two or more circular RNAs, each encoding a reprogramming factor selected from Oct3/4, Klf4, Sox2, Nanog, and Lin28 (e.g., Lin28A or Lin28B). In some embodiments, the composition comprises two or more circular RNAs, each encoding a reprogramming factor selected from Oct3/4, Klf4, Sox2, and c-Myc. In some embodiments, the composition comprises two or more circular RNAs, each encoding a reprogramming factor selected from Oct3/4, Klf4, and Sox2. In some embodiments, the composition comprises two or more circular RNAs, each encoding a reprogramming factor selected from the combinations provided in Table 3. In Table 3, each row represents a different combination of circular RNAs, wherein “X” indicates that the circular RNA is contacted with the cell. For example, in combination no. 1, the composition comprises a circular RNA encoding Oct3/4 and a circular RNA encoding Klf4. In combination no. 104, the composition comprises a circular RNAs encoding Oct3/4, Klf4, Sox2, and Nanog, Lin28 (e.g., Lin28A or Lin28B), and L-Myc.

TABLE 3
Exemplary reprogramming factor combinations

| | | | | | Circ | | |
|---|---|---|---|---|---|---|---|
| Combination | Circ | | | Circ | Lin28 (A | Circ | Circ |
| No. | Oct3/4 | circKlf4 | circSox2 | Nanog | or B) | C-Myc | L-Myc |
| 1 | X | X | | | | | |
| 2 | X | | X | | | | |
| 3 | X | | | X | | | |
| 4 | X | | | | X | | |
| 5 | X | | | | | X | |
| 6 | X | | | | | | X |
| 7 | | X | X | | | | |
| 8 | | X | | X | | | |
| 9 | | X | | | X | | |
| 10 | | X | | | | X | |
| 11 | | X | | | | | X |
| 12 | | | X | X | | | |
| 13 | | | X | | X | | |
| 14 | | | X | | | X | |
| 15 | | | X | | | | X |
| 16 | | | | X | X | | |
| 17 | | | | X | | X | |
| 18 | | | | X | | | X |
| 19 | | | | | X | X | |
| 20 | | | | | X | | X |
| 21 | | | | | | X | X |
| 22 | X | X | X | | | | |
| 23 | X | X | | X | | | |
| 24 | X | X | | | X | | |
| 25 | X | X | | | | X | |
| 26 | X | X | | | | | X |
| 27 | X | | X | X | | | |
| 28 | X | | X | | X | | |
| 29 | X | | X | | | X | |
| 30 | X | | X | | | | X |
| 31 | X | | | X | X | | |
| 32 | X | | | X | | X | |
| 33 | X | | | X | | | X |
| 34 | X | | | | X | X | |
| 35 | X | | | | X | | X |
| 36 | X | | | | | X | X |
| 37 | | X | X | X | | | |
| 38 | | X | X | | X | | |
| 39 | | X | X | | | X | |
| 40 | | X | X | | | | X |
| 41 | | X | | X | X | | |
| 42 | | X | | X | | X | |
| 43 | | X | | X | | | X |
| 44 | | X | | | X | X | |
| 45 | | X | | | X | | X |
| 46 | | X | | | | X | X |
| 47 | | | X | X | X | | |
| 48 | | | X | X | | X | |
| 49 | | | X | X | | | X |
| 50 | | | X | | X | X | |
| 51 | | | X | | X | | X |
| 52 | | | X | | | X | X |
| 53 | | | | X | X | X | |
| 54 | | | | X | X | | X |
| 55 | | | | X | | X | X |
| 56 | | | | | X | X | X |
| 57 | X | X | X | X | | | |
| 58 | X | X | X | | X | | |
| 59 | X | X | X | | | X | |
| 60 | X | X | X | | | | X |
| 61 | X | X | | X | X | | |
| 62 | X | X | | X | | X | |
| 63 | X | X | | X | | | X |
| 64 | X | X | | | X | X | |
| 65 | X | X | | | X | | X |
| 66 | X | X | | | | X | X |
| 67 | X | | X | X | X | | |
| 68 | X | | X | X | | X | |
| 69 | X | | X | X | | | X |
| 70 | X | | X | | X | X | |
| 71 | X | | X | | X | | X |
| 72 | X | | X | | | X | X |
| 73 | X | | | X | X | X | |
| 74 | X | | | X | X | | X |
| 75 | X | | | X | | X | X |
| 76 | X | | | | X | X | X |
| 77 | | X | X | X | X | | |
| 78 | | X | X | X | | X | |
| 79 | | X | X | X | | | X |
| 80 | | X | | X | X | X | |
| 81 | | X | | X | X | | X |
| 82 | | X | | X | | X | X |
| 83 | | X | | | X | X | X |
| 84 | | | X | X | X | X | |
| 85 | | | X | X | X | | X |
| 86 | | | | X | X | X | X |
| 87 | X | X | X | X | X | | |
| 88 | X | X | X | X | | X | |
| 89 | X | X | X | X | | | X |
| 90 | X | X | X | | X | X | |
| 91 | X | X | X | | X | | X |
| 92 | X | X | X | | | X | X |
| 93 | X | | X | X | X | X | |
| 94 | X | | X | X | X | | X |
| 95 | X | X | | X | X | X | |
| 96 | X | X | | X | X | | X |
| 97 | X | X | | | X | X | X |
| 98 | | X | X | X | X | X | |
| 99 | | X | | X | X | X | X |
| 100 | X | X | X | X | X | X | |
| 101 | X | | X | X | X | X | X |
| 102 | X | X | | X | X | X | X |
| 103 | X | X | X | | X | X | X |
| 104 | X | X | X | X | X | | X |
| 105 | | X | X | X | X | X | X |
| 106 | X | X | X | X | X | X | X |

In some embodiments, the composition comprises two circular RNAs, one of which encodes Oct3/4, and the other of which encodes a reprogramming factor selected from the group consisting of Klf4, Sox2, Lin28 (e.g., Lin28A or Lin28B), Nanog, and c-Myc or L-Myc. In some embodiments, the composition comprises two circular RNAs each encoding a reprogramming factor selected from Oct3/4 and Lin28 (e.g., Lin28A or Lin28B), or fragments or variants thereof. In some embodiments, the composition comprises two circular RNAs each encoding a reprogramming factor selected from Oct3/4 and Sox2, or fragments or variants thereof.

In some embodiments, the composition comprises three circular RNAs, each encoding a reprogramming factor selected from the group consisting of Oct3/4, Klf4, Sox2 or fragments or variants thereof. In some embodiments, the composition does not comprise a circular RNA encoding a reprogramming factor selected from Nanog, Lin28 (e.g., Lin28A or Lin28B), and/or c-Myc or fragments or variants thereof. In some embodiments, the composition consists of three circular RNAs, each encoding a reprogramming factor selected from the group consisting of Oct3/4, Klf4, Sox2 or fragments or variants thereof. In some embodiments, the composition comprises three circular RNAs, each encoding a reprogramming factor selected from the group consisting of Oct3/4, Klf4, Lin28 (e.g., Lin28A or Lin28B) or fragments or variants thereof. In some embodiments, the composition does not comprise a circular RNA encoding a reprogramming factor selected from Nanog, Sox2, and/or c-Myc or fragments or variants thereof. In some embodiments, the composition consists of three circular RNAs, each encoding a reprogramming factor selected from the group consisting of Oct3/4, Klf4, Lin28 (e.g., Lin28A or Lin28B) or fragments or variants thereof. In some embodiments, the composition comprises three circular RNAs, each encoding a reprogramming factor selected from the group consisting of Oct3/4, Sox2, Lin28 (e.g., Lin28A or Lin28B) or fragments or variants thereof. In some embodiments, the composition does not comprise a circular RNA encoding a reprogramming factor selected from Nanog, Klf4, and/or c-Myc or fragments or variants thereof. In some embodiments, the composition consists of three circular RNAs, each encoding a reprogramming factor selected from the group consisting of Oct3/4, Sox2, Lin28 (e.g., Lin28A or Lin28B) or fragments or variants thereof.

In some embodiments, the composition comprises four circular RNAs, each encoding a reprogramming factor selected from the group consisting of Oct3/4, Klf4, Sox2, and c-Myc or fragments or variants thereof. In some embodiments, the composition does not comprise a circular RNA encoding a reprogramming factor selected from Nanog and/or Lin28 (e.g., Lin28A or Lin28B) or fragments or variants thereof. In some embodiments, the composition consists of four circular RNAs, each encoding a reprogramming factor selected from the group consisting of Oct3/4, Klf4, Sox2, and c-Myc or fragments or variants thereof. In some embodiments, the composition comprises five circular RNAs, each encoding a reprogramming factor selected from the group consisting of Oct3/4, Klf4, Sox2, Nanog, and/or Lin28 (e.g., Lin28A or Lin28B) or fragments or variants thereof. In some embodiments, the composition does not comprise a circular RNA encoding c-Myc or fragments or variants thereof. In some embodiments, the composition consists of five circular RNAs, each encoding a reprogramming factor selected from the group consisting of Oct3/4, Klf4, Sox2, Nanog, and/or Lin28 (e.g., Lin28A or Lin28B) or fragments or variants thereof. In some embodiments, the composition comprises six circular RNAs, each encoding a reprogramming factor selected from the group consisting of Oct3/4, Klf4, Sox2, Nanog, Lin28 (e.g., Lin28A or Lin28B), c-Myc or fragments or variants thereof. In some embodiments, the composition consists of six circular RNAs, each encoding a reprogramming factor selected from the group consisting of Oct3/4, Klf4, Sox2, Nanog, Lin28 (e.g., Lin28A or Lin28B), c-Myc or fragments or variants thereof.

In some embodiments, the composition comprises one or more circular RNAs encoding one or more reprogramming factors and further comprises at least one additional ancillary factor that can aid in cellular reprogramming. In some embodiments, the composition further comprises an RNA polynucleotide (e.g., linear RNA polynucleotides or circular RNAs) encoding the ancillary factor. In some embodiments, the ancillary factor is selected from an miRNA (e.g., miRNA302 family (e.g., miR302d, miR302a, miR302c and miR302b), miR367, miR766, miR200c, miR369, miR372, Let7, miR19a/b), a long non-coding RNA (e.g., LINCRNA-ROR), and exogenous circRNA (e.g., circBIRC6, circCORO1C, or circMAN1A2. circBIRC6, circCORO1C and circMAN1A2), or a viral protein (e.g., those described in Table 2). In some embodiments, the composition consists of one or more circular RNAs encoding one or more reprogramming factors and does not comprise any additional RNA polynucleotide (e.g., linear RNA polynucleotides or circular RNAs) encoding an ancillary factor that can aid in cellular reprogramming.

### Methods for Reprogramming Cells

In some embodiments, the present disclosure provides methods for reprogramming somatic cells. In some embodiments, the present disclosure provides methods for reprogramming somatic cells to produce iPSCs. In particular embodiments, the present disclosure provides methods for reprogramming somatic cells in suspension culture to produce iPSCs. In some embodiments, a method of producing an iPSC comprises contacting a somatic cell (e.g., contacting the cell in a suspension culture) with at least one of the circular RNAs or composition thereof described herein and maintaining the cell under conditions under which a reprogrammed cell (e.g., an iPSC) is obtained. Herein, the circular RNAs that are introduced into the cells according to the methods described herein are exogenous to the cells. In other words, the circular RNAs introduced to the cells do not naturally occur in the cells to which they are introduced.

In some embodiments, the somatic cell is a prokaryotic cell. In some embodiments, the somatic cell is a eukaryotic cell. In some embodiments, the somatic cell is an animal cell. In some embodiments, the somatic cell is a mammalian cell (e.g., a murine, bovine, simian, porcine, equine, ovine, or human cell). In some embodiments, the somatic cell is a human cell. In some embodiments, the somatic cell is a yeast, fungi, or plant cell.

In some embodiments, the somatic cell is a fibroblast, a peripheral blood-derived cell, an endothelial progenitor cell, a cord-blood derived cell, a hepatocyte, a keratinocyte, a melanocyte, an adipose-tissue derived cell, or a urine-derived cell (e.g., a renal epithelial progenitor cell). In some embodiments, the cell is an epithelial cell, an endothelial cell, a neuronal cell, an adipose cell, a cardiac cell, a skeletal muscle cell, an immune cell, a hepatic cell, a splenic cell, a lung cell, a circulating blood cell, a gastrointestinal cell, a renal cell, a chondrocyte, an ocular cell, a neural cell, a cell of the central nervous system, an osteocyte, a bone marrow cell, a progenitor cell, or a pancreatic cell. In some embodiments, the cell is isolated from any somatic tissue including, but not limited to, brain, liver, lung, gut, stomach, intestine, fat, muscle, uterus, skin, spleen, endocrine organ, bone, etc.

In some embodiments, the somatic cell is an adherent cell. In some embodiments, the cell is a non-adherent cell. In some embodiments, the somatic cell reprogrammed in a suspension cell culture. In some embodiments, the somatic cell is a blood cell (e.g., a T cell, a B cell, an NK cell, an NKT cell, a peripheral blood mononucleocyte, or a cord blood mononucleocyte). In some embodiments, the blood cell is reprogrammed in a suspension cell culture. In some embodiments, the somatic cell is a T cell or an NK cell. In some embodiments, the T cell or an NK cell is reprogrammed in a suspension cell culture. In some embodiments, the somatic cell is a CD34+ cell. In some embodiments, the CD34+ cell is reprogrammed in a suspension cell culture.

In some embodiments, the somatic cell is contacted once with a circular RNA or composition thereof. In some embodiments, the somatic cell is contacted once with a plurality of different circular RNAs or one or more compositions thereof. In some embodiments, the somatic cell is contacted once with at least two different circular RNAs or one or more compositions thereof (e.g., 2, 3, 4, 5, or 6 circular RNAs or one or more compositions thereof). In some embodiments, the contacting is performed in suspension culture. In some embodiments the cell is contacted with the circular RNA more than once (e.g., 2, 3, 4, 5, 6, 7, 8, 9, or 10 times). In some embodiments the cell is contacted with a plurality of circular RNA more than once (e.g., 2, 3, 4, 5, 6, 7, 8, 9, or 10 times). In some embodiments, the somatic cell is contacted with the circular RNA or composition thereof from 2 to 6 times. In some embodiments, the somatic cell is contacted with the circular RNA or composition thereof twice. In some embodiments, the somatic cell is contacted with the circular RNA or composition thereof three times. In some embodiments, the somatic cell is contacted with the circular RNA or composition thereof four times. In some embodiments, the somatic cell is contacted with the circular RNA or composition thereof fewer than four times. In some embodiments, the contacting is performed at effective intervals. The effective intervals may be, for example, once per day, once every other day, once every three days, once per week, once every two weeks, or once per month. In some embodiments, the circular RNA is contacted with the cells for the duration of the reprogramming process, such that the contact is continuous throughout the reprogramming process. In some embodiments, a somatic cell is contacted (e.g., in a suspension culture) with a plurality of circular RNAs one or more times, wherein the number of contacts differs for at least two circular RNAs. For example, in some embodiments, a somatic cell is contacted (e.g., in a suspension culture) with a plurality of circular RNAs, wherein the cell is contacted with at least one of the circular RNAs more than once and the cell is contacted with at least one other of the circular RNAs only once. In some embodiments, a somatic cell is contacted (e.g., in a suspension culture) with a plurality of circular RNAs, wherein the cell is contacted with at least one of the circular RNAs more than twice and the cell is contacted with at least one other of the circular RNAs only once or only twice.

In some embodiments, the contacting comprises transfecting a circular RNA into the cell. In various embodiments, the methods described herein comprise directly contacting the cell with the circRNA or composition thereof. In other words, circular RNAs synthesized or manufactured in vitro are contacted directly with the cell (and thereby introduced into the cell) without the aid of a viral vector or DNA plasmid.

In some embodiments, the methods of reprogramming cells (e.g., methods of producing iPSCs) comprise contacting a somatic cell with a circular RNA or composition thereof, wherein the RNA is present at a concentration of at least 3 pg/cell. In some embodiments, the RNA is present at a concentration from about 3 pg/cell to about 15 pg/cell, about 4 pg/cell to about 15 pg/cell, about 5 pg/cell to about 15 pg/cell, about 6 pg/cell to about 15 pg/cell, about 7 pg/cell to about 15 pg/cell, about 8 pg/cell to about 15 pg/cell, about 9 pg/cell to about 15 pg/cell, about 10 pg/cell to about 15 pg/cell, about 11 pg/cell to about 15 pg/cell, about 12 pg/cell to about 15 pg/cell, about 13 pg/cell to about 15 pg/cell, or about 14 pg/cell to about 15 pg/cell. In some embodiments, the RNA is present at a concentration from about 3 pg/cell to about 14 pg/cell, about 4 pg/cell to about 14 pg/cell, about 5 pg/cell to about 4 pg/cell, about 6 pg/cell to about 14 pg/cell, about 7 pg/cell to about 14 pg/cell, about 8 pg/cell to about 14 pg/cell, about 9 pg/cell to about 14 pg/cell, about 10 pg/cell to about 14 pg/cell, about 11 pg/cell to about 14 pg/cell, about 12 pg/cell to about 14 pg/cell, or about 13 pg/cell to about 14 pg/cell. In some embodiments, the RNA is present at a concentration from about 3 pg/cell to about 13 pg/cell, about 4 pg/cell to about 13 pg/cell, about 5 pg/cell to about 13 pg/cell, about 6 pg/cell to about 13 pg/cell, about 7 pg/cell to about 13 pg/cell, about 8 pg/cell to about 13 pg/cell, about 9 pg/cell to about 13 pg/cell, about 10 pg/cell to about 13 pg/cell, about 11 pg/cell to about 13 pg/cell, or about 12 pg/cell to about 13 pg/cell. In some embodiments, the RNA is present at a concentration from about 3 pg/cell to about 12 pg/cell, about 4 pg/cell to about 12 pg/cell, about 5 pg/cell to about 12 pg/cell, about 6 pg/cell to about 12 pg/cell, about 7 pg/cell to about 12 pg/cell, about 8 pg/cell to about 12 pg/cell, about 9 pg/cell to about 12 pg/cell, about 10 pg/cell to about 12 pg/cell, or about 11 pg/cell to about 12 pg/cell. In some embodiments, the RNA is present at a concentration from about 3 pg/cell to about 11 pg/cell, about 4 pg/cell to about 11 pg/cell, about 5 pg/cell to about 11 pg/cell, about 6 pg/cell to about 11 pg/cell, about 7 pg/cell to about 11 pg/cell, about 8 pg/cell to about 11 pg/cell, about 9 pg/cell to about 11 pg/cell, or about 10 pg/cell to about 11 pg/cell. In some embodiments, the RNA is present at a concentration from about 3 pg/cell to about 10 pg/cell, about 4 pg/cell to about 10 pg/cell, about 5 pg/cell to about 10 pg/cell, about 6 pg/cell to about 10 pg/cell, about 7 pg/cell to about 10 pg/cell, about 8 pg/cell to about 10 pg/cell, or about 9 pg/cell to about 10 pg/cell. In some embodiments, the RNA is present at a concentration from about 3 pg/cell to about 9 pg/cell, about 4 pg/cell to about 9 pg/cell, about 5 pg/cell to about 9 pg/cell, about 6 pg/cell to about 9 pg/cell, about 7 pg/cell to about 9 pg/cell, or about 8 pg/cell to about 9 pg/cell. In some embodiments, the RNA is present at a concentration from about 3 pg/cell to about 8 pg/cell, about 4 pg/cell to about 8 pg/cell, about 5 pg/cell to about 8 pg/cell, about 6 pg/cell to about 8 pg/cell, or about 7 pg/cell to about 8 pg/cell. In some embodiments, the RNA is present at a concentration from about 3 pg/cell to about 7 pg/cell, about 4 pg/cell to about 7 pg/cell, about 5 pg/cell to about 7 pg/cell, or about 6 pg/cell to about 7 pg/cell. In some embodiments, the RNA is present at a concentration from about 3 pg/cell to about 6 pg/cell, about 4 pg/cell to about 6 pg/cell, or about 5 pg/cell to about 6 pg/cell. In some embodiments, the RNA is present at a concentration from about 3 pg/cell to about 5 pg/cell, or about 4 pg/cell to about 5 pg/cell. In some embodiments, the RNA is present at a concentration from about 3 pg/cell to about 4 pg/cell. In some embodiments, the RNA is present at a concentration of about 1 pg/cell, 2 pg/cell, 3 pg/cell, 4 pg/cell, 5 pg/cell, 6 pg/cell, 7 pg/cell, 8 pg/cell, 9 pg/cell, 10 pg/cell, 11 pg/cell, 12 pg/cell, 13 pg/cell, 14 pg/cell, or about 15 pg/cell.

In some embodiments, the circular RNA is transfected into the cell using lipid-mediated transfection. Lipid-mediated transfection stimulates active uptake of nucleic acids by endocytosis. An exemplary lipid-mediated transfection reagent is Lipofectamine® (e.g., Lipofectamine® RNAiMAX®, from ThermoFisher®). In some embodiments, a method for transfecting a cell comprises the steps of (i) diluting the RNA and the transfection reagent in separate tubes, (ii) combining the RNA with the transfection reagent to form complexes, (iii) adding the complexes to the cells, (iv) assaying the cells for protein expression. Detection of protein expression in cells can be achieved by several techniques including Western blot analysis, immunocytochemistry, and fluorescence-mediated detection (e.g., FACS), among others.

In some embodiments, the contacting comprises electroporating a circular RNA or composition thereof into the cell. Electroporation delivers nucleic acids by transiently opening holes in the cell membrane while the cell is in a solution in which the nucleic acid is present at high concentration. In some embodiments, the electroporation uses the Neon® electroporation system.

In some embodiments, the contacting comprises incubating the cells with circRNA-LNP complexes.

In some embodiments, the contacting comprises one or more techniques such as ballistic transfection (i.e., gene gun or biolistic transfection), magnetofection, peptide-mediated transfection (either non-covalent peptide/RNA nanoparticle-based transfection such as the N-TER™ Transfection System from Sigma-Aldrich or by covalent attachment of the peptide to the RNA), and/or microinjection. Combinations of these techniques used in succession or simultaneously can also be used.

In some embodiments, a method of reprogramming a cell (e.g. a method of producing an iPSC) comprises contacting a somatic cell with at least one circular RNA encoding a reprogramming factor (e.g., a transcription factor), and maintaining the cell under conditions under which a reprogrammed cell (e.g. an iPSC) is obtained. The reprogramming factor may be, for example, any of the reprogramming factors shown in Table 1. In some embodiments, the cell is contacted with multiple circular RNAs, wherein each circular RNA encodes a reprogramming factor selected from the reprogramming factors shown in Table 1.

In some embodiments, the present disclosure provides a method of reprogramming a cell, wherein the cell is contacted with a combination of circular RNAs according to any one of combinations 1-106 of as shown in Table 3A below.

TABLE 3A
Exemplary reprogramming factor combinations

| | | | | | Circ | | |
|---|---|---|---|---|---|---|---|
| Combination | Circ | | | Circ | Lin28 (A | Circ | Circ |
| No. | Oct3/4 | circKlf4 | circSox2 | Nanog | or B) | C-Myc | L-Myc |
| 1 | X | X | | | | | |
| 2 | X | | X | | | | |
| 3 | X | | | X | | | |
| 4 | X | | | | X | | |
| 5 | X | | | | | X | |
| 6 | X | | | | | | X |
| 7 | | X | X | | | | |
| 8 | | X | | X | | | |
| 9 | | X | | | X | | |
| 10 | | X | | | | X | |
| 11 | | X | | | | | X |
| 12 | | | X | X | | | |
| 13 | | | X | | X | | |
| 14 | | | X | | | X | |
| 15 | | | X | | | | X |
| 16 | | | | X | X | | |
| 17 | | | | X | | X | |
| 18 | | | | X | | | X |
| 19 | | | | | X | X | |
| 20 | | | | | X | | X |
| 21 | | | | | | X | X |
| 22 | X | X | X | | | | |
| 23 | X | X | | X | | | |
| 24 | X | X | | | X | | |
| 25 | X | X | | | | X | |
| 26 | X | X | | | | | X |
| 27 | X | | X | X | | | |
| 28 | X | | X | | X | | |
| 29 | X | | X | | | X | |
| 30 | X | | X | | | | X |
| 31 | X | | | X | X | | |
| 32 | X | | | X | | X | |
| 33 | X | | | X | | | X |
| 34 | X | | | | X | X | |
| 35 | X | | | | X | | X |
| 36 | X | | | | | X | X |
| 37 | | X | X | X | | | |
| 38 | | X | X | | X | | |
| 39 | | X | X | | | X | |
| 40 | | X | X | | | | X |
| 41 | | X | | X | X | | |
| 42 | | X | | X | | X | |
| 43 | | X | | X | | | X |
| 44 | | X | | | X | X | |
| 45 | | X | | | X | | X |
| 46 | | X | | | | X | X |
| 47 | | | X | X | X | | |
| 48 | | | X | X | | X | |
| 49 | | | X | X | | | X |
| 50 | | | X | | X | X | |
| 51 | | | X | | X | | X |
| 52 | | | X | | | X | X |
| 53 | | | | X | X | X | |
| 54 | | | | X | X | | X |
| 55 | | | | X | | X | X |
| 56 | | | | | X | X | X |
| 57 | X | X | X | X | | | |
| 58 | X | X | X | | X | | |
| 59 | X | X | X | | | X | |
| 60 | X | X | X | | | | X |
| 61 | X | X | | X | X | | |
| 62 | X | X | | X | | X | |
| 63 | X | X | | X | | | X |
| 64 | X | X | | | X | X | |
| 65 | X | X | | | X | | X |
| 66 | X | X | | | | X | X |
| 67 | X | | X | X | X | | |
| 68 | X | | X | X | | X | |
| 69 | X | | X | X | | | X |
| 70 | X | | X | | X | X | |
| 71 | X | | X | | X | | X |
| 72 | X | | X | | | X | X |
| 73 | X | | | X | X | X | |
| 74 | X | | | X | X | | X |
| 75 | X | | | X | | X | X |
| 76 | X | | | | X | X | X |
| 77 | | X | X | X | X | | |
| 78 | | X | X | X | | X | |
| 79 | | X | X | X | | | X |
| 80 | | X | | X | X | X | |
| 81 | | X | | X | X | | X |
| 82 | | X | | X | | X | X |
| 83 | | X | | | X | X | X |
| 84 | | | X | X | X | X | |
| 85 | | | X | X | X | | X |
| 86 | | | | X | X | X | X |
| 87 | X | X | X | X | X | | |
| 88 | X | X | X | X | | X | |
| 89 | X | | X | X | | | X |
| 90 | X | X | X | | X | X | |
| 91 | X | X | X | | X | | X |
| 92 | X | X | X | | | X | X |
| 93 | X | | X | X | X | X | |
| 94 | X | | X | X | X | | X |
| 95 | X | X | | X | X | X | |
| 96 | X | X | | X | X | | X |
| 97 | X | X | | | X | X | X |
| 98 | | X | X | X | X | X | |
| 99 | | X | | X | X | X | X |
| 100 | X | X | X | X | X | X | |
| 101 | X | | X | X | X | X | X |
| 102 | X | X | | X | X | X | X |
| 103 | X | X | X | | X | X | X |
| 104 | X | X | X | X | X | | X |
| 105 | | X | X | X | X | X | X |
| 106 | X | X | X | X | X | X | X |

In some embodiments, the present disclosure provides a method of reprogramming a cell in suspension comprising contacting the cell with a combination of circular RNAs according to any one of combinations 1-106 as shown in Table 3A above. In some embodiments, the circular RNAs of such a combination (i.e., one of combinations 1-106) are contacted to the cell in suspension a plurality of times. In some embodiments, at least one of circular RNAs of such a combination (i.e., one of combinations 1-106) are contacted to the cell in suspension a plurality of times and at least one of the circular RNAs of such a combination is contacted to the cell in suspension one time. In some embodiments, one or more of the circular RNAs of such a combination is contacted with the cells in suspension one time, two times, three times, or four times.

In some embodiments, the present disclosure provides a method of reprogramming a blood cell (e.g., T cells, B cells, NK cells, NKT cells, peripheral blood mononucleocytes, cord blood mononucleocytes, CD34+ cells) comprising contacting the cell with a combination of circular RNAs according to any one of combinations 1-106 of as shown in Table 3A above. In some embodiments, the blood cell is a T cell. In some embodiments, the circular RNAs of such a combination (i.e., one of combinations 1-106) are contacted to the T cell in suspension a plurality of times. In some embodiments, at least one of circular RNAs of such a combination (i.e., one of combinations 1-106) are contacted to the T cell in suspension a plurality of times and at least one other of the circular RNAs of such a combination is contacted to the T cell in suspension one time. In some embodiments, one or more of the circular RNAs of such a combination is contacted with the T cells in suspension one time, two times, three times, or four times.

In some embodiments, the blood cell is an NK cell. In some embodiments, the circular RNAs of such a combination (i.e., one of combinations 1-106) are contacted to the NK cell in suspension a plurality of times. In some embodiments, at least one of circular RNAs of such a combination (i.e., one of combinations 1-106) are contacted to the NK cell in suspension a plurality of times and at least one of the circular RNAs of such a combination is contacted to the NK cell in suspension one time. In some embodiments, one or more of the circular RNAs of such a combination is contacted with the NK cells in suspension one time, two times, three times, or four times.

In some embodiments, the present disclosure provides a method of reprogramming a CD34+ cell in suspension comprising contacting the CD34+ cell with a combination of circular RNAs according to any one of combinations 1-106 of as shown in Table 3A above. In some embodiments, the circular RNAs of such a combination (i.e., one of combinations 1-106) are contacted to the CD34+ cell in suspension a plurality of times. In some embodiments, at least one of circular RNAs of such a combination (i.e., one of combinations 1-106) are contacted to the CD34+ cell in suspension a plurality of times and at least one other of the circular RNAs of such a combination is contacted to the CD34+ cell in suspension one time. In some embodiments, one or more of the circular RNAs of such a combination is contacted with the CD34+ cells in suspension one time, two times, three times, or four times.

In some embodiments, the reprogramming factor is Oct3/4, Klf4, Sox2, Nanog, Lin28 (e.g., Lin28A or Lin28B), c-Myc, or L-Myc. In some embodiments, the reprogramming factor is Oct3/4. In some embodiments, the reprogramming factor is Klf4. In some embodiments, the reprogramming factor is Sox2. In some embodiments, the reprogramming factor is Nanog. In some embodiments, the reprogramming factor is Lin28 (e.g., Lin28A or Lin28B). In some embodiments, the reprogramming factor is c-Myc. In some embodiments, the reprogramming factor is L-Myc.

In some embodiments, a method of reprogramming a cell (e.g. a method of producing an iPSC) comprises contacting a somatic cell with more than one circular RNA, wherein each circular RNA encodes a reprogramming factor selected from Oct3/4, Klf4, Sox2, Nanog, Lin28 (e.g., Lin28A or Lin28B), c-Myc, and L-Myc, and maintaining the cell under conditions under which a reprogrammed cell (e.g., an iPSC) is obtained. In some embodiments, the cell is contacted with at least 2, at least 3, at least 4, at least 5, at least 6, at least 7, at least 8, at least 9, at least 10, or more circular RNAs, each encoding a reprogramming factor selected from Oct3/4, Klf4, Sox2, Nanog, Lin28 (e.g., Lin28A or Lin28B), c-Myc, or L-Myc.

In some embodiments, a method of reprogramming a cell (e.g., a method of producing an iPSC) comprises contacting a somatic cell with 6 circular RNAs encoding the reprogramming factors Oct3/4, Klf4, Sox2, Nanog, Lin28 (e.g., Lin28A or Lin28B), and c-Myc. In some embodiments, a method of reprogramming a cell (e.g., a method of producing an iPSC) comprises contacting a somatic cell with 6 circular RNAs encoding the reprogramming factors Oct3/4, Klf4, Sox2, Nanog, Lin28 (e.g., Lin28A or Lin28B), and L-Myc.

In some embodiments, a method of reprogramming a cell (e.g., a method of producing an iPSC) comprises contacting a somatic cell with 5 circular RNAs encoding the reprogramming factors Oct3/4, Klf4, Sox2, Nanog, and Lin28 (e.g., Lin28A or Lin28B). In some embodiments, the method does not comprise contacting the somatic cell with a circular RNA encoding c-Myc or L-Myc.

In some embodiments, a method of reprogramming a cell (e.g., a method of producing an iPSC) comprises contacting a somatic cell with 4 circular RNAs encoding the reprogramming factors Oct3/4, Klf4, Sox2, and c-Myc. In some embodiments, a method of reprogramming a cell (e.g., a method of producing an iPSC) comprises contacting a somatic cell with 4 circular RNAs encoding the reprogramming factors Oct3/4, Klf4, Sox2, and L-Myc. In some embodiments, the method does not comprise contacting the somatic cell with a circular RNA encoding Nanog and/or Lin28 (e.g., Lin28A or Lin28B).

In some embodiments, a method of reprogramming a cell (e.g., a method of producing an iPSC) comprises contacting a somatic cell with 3 circular RNAs encoding the reprogramming factors Oct3/4, Klf4, and Sox2. In some embodiments, the method does not comprise contacting the somatic cell with a circular RNA encoding Nanog, Lin28 (e.g., Lin28A or Lin28B), c-Myc, and/or L-myc.

In some embodiments, the method of reprogramming a somatic cell (e.g., producing an iPSC) comprises contacting the somatic cell with one or more circular RNAs encoding one or more reprogramming factors and further comprises contacting the cell with at least one additional RNA polynucleotide (e.g., linear RNA polynucleotides or circular RNAs) encoding an ancillary factor that can aid in cellular reprogramming. In some embodiments, the ancillary factor is selected from an miRNA (e.g., miRNA302(a-d), miR367, miR766, miR200c, miR369, miR372, Let7, miR19a/b), a long non-coding RNA (e.g., LINCRNA-ROR), and exogenous circRNA (e.g., circBIRC6, circCORO1C, or circMAN1A2. circBIRC6, circCORO1C and circMAN1A2), or a viral protein (e.g., those described in Table 2). In some embodiments, the method of reprogramming a somatic cell (e.g., producing an iPSC) comprises contacting the somatic cell with one or more circular RNAs encoding one or more reprogramming factors and further comprises contacting the cell with at least one additional exogenous factor selected from vitamin C, valproic acid, CHIR99021, Parnate, SB431542, PD0325901, BIX-01294, Lithium Maxadilan, 8-Br-CAMP, A-83-01, Tiazovivin, Y-27632, EPZ004777, and DAPT.

In some embodiments, the method of reprogramming a somatic cell (e.g., producing an iPSC) comprises contacting the somatic cell with one or more circular RNAs encoding one or more reprogramming factors and does not comprise contacting the cell with any additional RNA polynucleotide (e.g., linear RNA polynucleotides or circular RNAs) encoding an ancillary factor that can aid in cellular reprogramming. In some embodiments, the method of reprogramming a somatic cell (e.g., producing an iPSC) comprises contacting the somatic cell with one or more circular RNAs encoding one or more reprogramming factors and does not comprise contacting the cell with an ancillary factor that aids in cellular reprogramming.

Some illustrative combinations of RNAs for use in a method of reprogramming a cell are shown below in Table 4. In Table 4, each row represents a different combination that may be contacted with a cell, wherein “X” indicates that the RNA is contacted with the cell. For example, in combination no. 1, the cell is contacted with a circular RNA encoding a reprogramming factor. In combination no. 15, the cell is contacted with a circular RNA encoding a reprogramming factor, a circular RNA that does not encode any protein or miRNA, a circular or linear RNA encoding a miRNA, and a circular or linear RNA encoding a viral protein.

TABLE 4
Combinations of RNAs for use in a method of reprogramming a cell

| | Circular RNA | | | |
|---|---|---|---|---|
| | encoding a | Circular or linear | | |
| | reprogramming | RNA that does not | Circular or linear | |
| | factor or | encode any protein | RNA encoding a | Circular or linear |
| | combination | or miRNA (e.g., | miRNA (e.g., | RNA encoding a |
| | thereof (See, | circBIRC6, | miR302d, miR302a, | viral protein (e.g., |
| Combination | e.g., Table 1 | circCORO1c, | miR302c, miR302b, | B18R, E3, K3 or |
| No. | and Table 3A) | circMAN1A2) | or miR367) | Table 2) |
| 1 | X | | | |
| 2 | | X | | |
| 3 | | | X | |
| 4 | | | | X |
| 5 | X | X | | |
| 6 | X | | X | |
| 7 | X | | | X |
| 8 | | X | X | |
| 9 | | X | | X |
| 10 | | | X | X |
| 11 | X | X | X | |
| 12 | X | X | | X |
| 13 | X | | X | X |
| 14 | | X | X | X |
| 15 | X | X | X | X |

The contacting may be performed by any of the methods described above, such as by transfection, electroporation, and/or the use of circRNA-LNP complexes. In some embodiments, the contacting comprises incubating the cell with one or more circular RNAs, such as circular RNAs encoding reprogramming factors.

In some embodiments, the methods for producing iPSCs may comprise maintaining the cell under conditions under which a reprogrammed iPSC is obtained. Such conditions are known to those of skill in the art and may vary by cell type. As one example, somatic cells may first be placed into a flask with the appropriate medium so that they are about 75% to about 90% confluent on the day that they are contacted with the circRNAs (Day 0). The cells may then be contacted with the circRNAs (e.g., by transfection). The transfected cells may be plated onto culture disks and incubated overnight. For the next 10-14 days, the media may be changed as required. In some embodiments, media may be supplemented with one or more additional agents to enhance cellular reprogramming. The cells may be monitored for the emergence of iPSC colonies, and iPSC colonies are picked and transferred into separate dishes for expansion.

To confirm the pluripotency of the iPSCs, isolated clones can be tested for the expression of one or more stem cell markers. Stem cell markers can be selected from, for example, Oct4, Lin28 (e.g., Lin28A or Lin28B), SOX2, SSEA4, SSEA3, TRA-1-81, TRA-1-60, CD9, Nanog, FbxI5, EcatI, EsgI, Eras, Gdf3, Fgf4, Cripto, DaxI, Zpf296, Slc2a3, RexI, UtfI, and Nat1. Methods for detecting the expression of such markers can include, for example, RT-PCR and immunological methods that detect the presence of the encoded polypeptides.

In some embodiments, the pluripotency of the cell is confirmed by measuring the ability of the cells to differentiate to cells of each of the three germ layers. In some embodiments, teratoma formation in immunocompromised rodents can be used to evaluate the pluripotent character of the isolated clones.

In some embodiments, circRNA reprogramming requires less frequent and/or a smaller number of transfections (as compared to linear RNA-based approaches) to achieve iPSC reprogramming. For example, circRNA reprogramming may require about 10%, about 20%, about 30%, about 40%, about 50%, about 60%, about 70%, about 80%, about 90%, or about 100% fewer transfections, as compared to linear RNA-based approaches, to achieve reprogramming.

In some embodiments, circRNA reprogramming results in enhanced reprogramming efficiency compared to linear RNA-based approaches. “Reprogramming efficiency” refers to a quantitative or qualitative measure of iPSC generation from a starting population of cells. Read-outs of reprogramming efficiency include quantitation of the number of iPSC colonies present at a particular timepoint during a reprogramming protocol (as an assessment of the rate of colony formation) or at the completion of a reprogramming protocol (as an assessment of the total number of iPSC colonies generated during a particular protocol). iPSC colonies can be identified quantitatively (such as by staining with markers of pluripotency and counting the number of stained cells) or qualitatively by assessment of morphological characteristics (e.g., tightly-packed cells with each cell in the colony having a more or less uniform shape and diameter, colonies comprising a clearly-defined border, and cells within iPSC colonies comprising a high nuclear to cytoplasmic ratio and prominent nucleoli). Reprogramming efficiency may also include an assessment of the relative maturity of iPSCs colonies between various reprogramming protocols. Maturation of iPSC colonies can be determined by the morphological characteristics noted above.

An increase in reprogramming efficiency refers to an increase in one or more read-outs of reprogramming efficiency when two or more reprogramming protocols are compared. For example, and as detailed in the Examples, reprogramming with circRNA-encoded reprogramming factors results in an increase in reprogramming efficiency compared to reprogramming with linear RNA-encoded reprogramming factors.

In some embodiments, increased reprogramming efficiency comprises an increase in the total number of iPSC colonies present at the end of a first reprogramming protocol compared to the total number of iPSC colonies present at the end of a second and/or third reprogramming protocol. In some embodiments, increased reprogramming efficiency comprises an increase in the total number of iPSC colonies present at a particular timepoint in a first reprogramming protocol compared to the total number of iPSC colonies present at the same timepoint in a second and/or third reprogramming protocol (i.e., an increase in the rate of iPSC colony formation).

### Methods for Transdifferentiating Cells

Provided herein are methods for transdifferentiating cells using circular RNAs. In some embodiments, a method of directly converting a cell from a first cell type to a second cell type comprises contacting the cell with a circular RNA or composition thereof as described herein and maintaining the cell under conditions under which the cell is converted to the second cell type. In some embodiments, the cell does not enter an intermediate pluripotent state. In some embodiments, the cell is converted directly from the first cell type to the second cell type, without becoming a progenitor cell.

In some embodiments, the circular RNA encodes one or more transdifferentiation factors that are capable of transdifferentiating cells from a first cell type to a second cell type. In some embodiments, a reprogramming factor (e.g., the reprogramming factors in Table 1) can function as a transdifferentiation factor in a given context or when used in combination with other transdifferentiation factors. For example, Sox2 functions as a reprogramming factor when used in reprogramming of a somatic cell to an iPSC, but functions as a transdifferentiation factor when used in transdifferentiation of first somatic cell (e.g., a fibroblast) to second somatic cell (e.g., a neural stem cell or a cardiomyocyte). In some embodiments, the circular RNA encodes MyoD, C/EBPα, C/EBPβ, Pdx1, Ngn3, Mafa, Pdx1, Hnf4a, Foxa1, Foxa2, Foxa3, AscI1 (also known as Mash1), Brn2, Myt1I, miR-124, Brn2, Myt1I, AscI1, Nurr1, Lmx1a, AscI1, Brn2, Myt1I, Lmx1a, FoxA2, Oct4, Sox2, Klf4 and c-Myc, Tbx5, Mef2c, Gata-4, and/or Mesp1. In some embodiments, the circular RNA encodes one or more reprogramming factors listed in Table 1.

In some embodiments, the first cell type is an iPSC. In some embodiments, the first cell type is a differentiated fibroblast.

In some embodiments, the second cell type is a muscle cell, a neuron, a cardiomyocyte, a hepatocyte, a renal cell, a chondrocyte, an osteocyte, an islet, a keratinocyte, a T-cell, or a NK-cell. In some embodiments, the second cell type is a muscle cell, a neuron, a cardiomyocyte, a hepatocyte, an islet cell, a keratinocyte, a T-cell, or a NK-cell.

In some embodiments, a method of directly converting a cell from a first cell type to a second cell type comprises contacting the cell with multiple circular RNAs, wherein each circular RNA encodes a transdifferentiation factor according to one of the combinations listed in Table 5.

In some embodiments, a method of directly converting a cell from a first cell type to a second cell type comprises contacting the cell with multiple circular RNAs wherein each circular RNA encodes a transdifferentiation factor listed in Table 5. In some embodiments, the cell is contacted with at least 2, at least 3, at least 4, at least 5, at least 6, at least 7, at least 8, at least 9, at least 10, or more circular RNAs.

In some embodiments, a method of directly converting a cell from a first cell type to a second cell type comprises contacting the cell with two circular RNAs and maintaining the cell under conditions under which the cell is converted to the second cell type. In some embodiments, the first and second circular RNAs each encode a transdifferentiation factor listed in Table 5, wherein the first and second circular RNAs do not encode the same transdifferentiation factor.

In some embodiments, a method of directly converting a cell from a first cell type to a second cell type comprises contacting the cell with three circular RNAs and maintaining the cell under conditions under which the cell is converted to the second cell type. In some embodiments, the first, second, and third circular RNAs each encode a transdifferentiation factor listed in Table 5, wherein the first, second, and third circular RNAs do not encode the same transdifferentiation factor.

In some embodiments, a method of directly converting a cell from a first cell type to a second cell type comprises contacting the cell with four circular RNAs and maintaining the cell under conditions under which the cell is converted to the second cell type. In some embodiments, the first, second, third, and fourth circular RNAs each encode a transdifferentiation factor listed in Table 5, wherein the first, second, third, and fourth circular RNAs do not encode the same transdifferentiation factor.

In some embodiments, a method of directly converting a cell from a first cell type to a second cell type comprises contacting the cell with five circular RNAs and maintaining the cell under conditions under which the cell is converted to the second cell type. In some embodiments, the first, second, third, fourth, and fifth circular RNAs each encode a transdifferentiation factor listed in Table 5, wherein the first, second, third, fourth and fifth circular RNAs do not encode the same transdifferentiation factor.

In some embodiments, a method of directly converting a cell from a first cell type to a second cell type comprises contacting the cell with six circular RNAs and maintaining the cell under conditions under which the cell is converted to the second cell type. In some embodiments, the first, second, third, fourth, fifth, and sixth circular RNAs each encode a transdifferentiation factor listed in Table 5, wherein the first, second, third, fourth, fifth and sixth circular RNAs do not encode the same transdifferentiation factor.

In some embodiments, a method of directly converting a cell from a first cell type to a second cell type comprises contacting the cell with seven circular RNAs and maintaining the cell under conditions under which the cell is converted to the second cell type. In some embodiments, the first, second, third, fourth, fifth, and sixth circular RNAs each encode a transdifferentiation factor listed in Table 5, wherein the first, second, third, fourth, fifth, and sixth circular RNAs do not encode the same transdifferentiation factor.

In some embodiments, a method of directly converting a cell from a first cell type to a second cell type comprises contacting the cell with multiple circular RNAs and maintaining the cell under conditions under which the cell is converted to the second cell type. In some embodiments, each of the circular RNAs each encode a transdifferentiation factor listed in Table 5, wherein none of the circular RNAs encode the same transdifferentiation factor.

In some embodiments, a cell is contacted with a circular RNA encoding one or more reprogramming factors listed in Table 5. In some embodiments, a method of directly converting a cell from a first cell type as shown in Table 5 to a second cell type as shown in Table 5 comprises contacting the cell with the circular RNA encoding one or more reprogramming factors listed in Table 5 and maintaining the cell under conditions under which the cell is converted to the second cell type. The first cell type may be, for example, any of the cell types listed in Table 5. The second cell type may be, for example, any of the cell types listed in Table 5.

In some embodiments, the present disclosure provides a composition comprising one or more circular RNAs, wherein each circular RNA encodes one or more of the transdifferentiation factors listed in Table 5. In some embodiments, the present disclosure provides a composition comprising a plurality of circular RNAs, each circular RNA encoding at least one transdifferentiation factor listed in Table 5.

In some embodiments, a method for transdifferentiating a cell comprises contacting a cell with one or more circular RNAs, wherein each of the circular RNAs encodes a transdifferentiation factor listed in Table 5. In some embodiments, a method for transdifferentiating a cell comprises contacting a cell with one or more circular RNAs, wherein each of the circular RNAs encodes a transdifferentiation factor listed in Table 5, and wherein the cell is any one of the “first cell type” listed in Table 5.

In some embodiments, a method for transdifferentiating a cell comprises contacting the first cell type listed Column A of any Combination No. shown in Table 5 with the corresponding transdifferentiation factor(s) shown in Column B of that same transdifferentiation combination to produce the second cell type shown in Column C of that same Combination No., wherein at least one transdifferentiation factor shown in Column B is encoded by a circular RNA. In some embodiments, all of the transdifferentiation factor(s) shown in Column B for a given transdifferentiation combination are encoded by one or more circularized RNA(s). In some embodiments, a first cell type is transdifferentiated to a second cell type using the transdifferentiation factors listed in Column B for any one of Combination Nos. 1-151. In some embodiments, the first cell type is any one of the cell types listed Column A for any one of Combination Nos. 1-151. In some embodiments, the second cell type is any one of the second cell types listed in Column C for any one of Combination Nos. 1-151.

TABLE 5
Exemplary transdifferentiation factors for converting
a cell from a first cell type to a second cell type

| Combination | Column A | Column B | Column C |
|---|---|---|---|
| No | First cell type | Transdifferentiation factor(s) | Second cell type |
| 1. | Fibroblast | MyoD | Myocyte |
| 2. | B-cell | C/EBPα, C/EBPβ | Macrophage |
| | Column A | Column B | Column C |
| 3. | Pancreatic duct cell | Pdx1 | Beta cell |
| 4. | Pancreatic exocrine cell | Ngn3, Mafa, Pdx1 | |
| 5. | Hepatocyte | Exendin-4, Pdx1 | |
| 6. | Fibroblast | Hnf4α, Foxa1 or Foxa2 or | Hepatocyte |
| | | Foxa3 | |
| 7. | Fibroblast | Ascl1 (also known as Mash1), | Neuron |
| | | Brn2, Myt1l, miR-124, Brn2, | |
| | | Myt1l | |
| 8. | Astrocyte | Pax6, neurogenin 2, Ascl1 | |
| 9. | Fibroblast | Ascl1, Nurr1, Lmx1a, Ascl1, | Dopaminergic neuron |
| | | Brn2, Myt1l, Lmx1a, FoxA2 | |
| 10. | Fibroblast | Oct4, Sox2, Klf4 and c-Myc, | Cardiomyocyte |
| | | Tbx5, Mef2c, Gata-4, Mesp1 | |
| 11. | Human Adult Dermal | Brn2, Mty1l, miRNA-124 | Neurons |
| | Fibroblast | | |
| 12. | Human Adult Peripheral | Ascl1, Brn2, Myt1l, Ngn2 | |
| | Blood Mononuclear | | |
| | Cells | | |
| 13. | Human Striatum | Ascl1, Brn2, Myt1l | |
| | Astrocytes | | |
| 14. | Murine Embryonic and | Ascl1, Brn2, Myt1l | |
| | Postnatal Fibroblasts | | |
| 15. | Human Neonatal | Foxa2, Hnf4α, C/EBPβ, c-Myc | Hepatocytes |
| | Fibroblasts | | |
| 16. | Human Embryonic | Hnf1α, Hnf4α, Foxa3 | |
| | Fibroblasts | | |
| 17. | Human Adult | ETV2 | Endothelial Cells |
| | Fibroblasts | | |
| 18. | Murine Amniotic Cells | Sox17 | |
| 19. | Human Newborn | Oct4, Sox2, KLF4, c-Myc | |
| | Dermal | bFGF, BME | |
| | and Lung Fibroblasts | | |
| 20. | Murine Embryonic | Myod1 | |
| | Fibroblasts | | |
| 21. | Human Dermal | Myod1 | |
| | Fibroblasts | SB431542, Chir99021, EGF, | |
| | | IGF1 | |
| 22. | Human Dermal | Cartilage-derived | Chondrocytes |
| | Fibroblasts | morphogenetic protein 1 | |
| 23. | Mouse Dermal | c-Myc, KLF4, Sox9 | |
| | Fibroblast | | |
| 24. | Murine Adult Pancreatic | Pdx1, Ngn3, Mafa | Pancreatic β-Cells |
| | Exocrine Cells | | |
| 25. | Human Pancreatic | MAPK, STAT3 | |
| | Exocrine Cells | | |
| 26. | Murine Cardiac | Gata4, Mef2c, Tbx5 | Cardiomyocytes |
| | Fibroblasts | | |
| 27. | Murine Cardiac | miRNA-1, miRNA-133, | |
| | Fibroblasts | miRNA-208, miRNA-499 | |
| 28. | Murine Myoblasts | Myod1 | Adipocytes |
| 29. | Murine Adipose Tissue- | Runx2 | |
| | Derived Stem Cells | | |
| 30. | Murine Preadipocytes | Runx2, MKP-1 | |
| 31. | Astrocytes | Pax6, Mash1, or Ngn2 | Glutamatergic |
| | | | neurons |
| 32. | Embryonic fibroblasts | Brn2, Ascl1, and Myt1l | Neuronal cells |
| | and Hepatocytes | | |
| 33. | Astrocytes | Dlx2; Dlx2 and Ascl1 | GABAergic neurons |
| 34. | Embryonic fibroblasts | Ascl1, Lmx1a, and Nurr1 | Dopaminergic neurons |
| | and Adult skin | | |
| | fibroblasts | | |
| 35. | Fetal fibroblasts and | BRN2, ASCL1, MYT1L, and | Neuronal cells |
| | Postnatal foreskin | NEUROD1 | |
| | fibroblasts | | |
| 36. | Embryonic fibroblasts | ASCL1, BRN2, MYT1L, | Neuronal cells |
| | and Postnatal | LMX1A, and FOXA2 | |
| | fibroblasts | | |
| 37. | Embryonic fibroblasts | Brn4/Pou3f4, Sox2, Klf4, c- | Neural stem cells |
| | | Myc, and E47/Tcf3 | |
| 38. | Embryonic fibroblasts | Sox2 | Neural stem cells |
| | and Fetal foreskin | | |
| | fibroblasts | | |
| 39. | Sertoli cells | Pax6, Ngn2, Hes1, Id1, Ascl1, | Neural stem cells |
| | | Brn2, c-Myc, and Klf4 | |
| 40. | Fibroblasts (IMR90 | MASH1, NGN2, SOX2, | Dopaminergic neurons |
| | cells) | NURR1, and PITX3 + A | |
| | | dominant-negative P53 | |
| 41. | Non-sensory cochlear | Ascl1; Ascl1 and Neurod | Neuronal cells |
| | epithelial cells | | |
| 42. | Astrocytes | Brn4 | Neuronal cells |
| 43. | Skin fibroblasts | Brn2, Sox2, and Foxa2 | Dopaminergic |
| | | | precursors |
| 44. | Adult skin fibroblasts | NEUROG2, SOX11, ISL1, and | Motor neurons |
| | | LHX3 | |
| 45. | Fibroblasts (3T6 cells) | Ascl1, Brn4, and Tcf3 | Neuronal cells |
| 46. | Umbilical cord blood | SOX2 and HMGA2 | Neural stem cells |
| | cells | | |
| 47. | Fibroblasts (3T6 cells) | Ascl1, Brn2, and Foxa1 | Neuronal cells |
| 48. | Resident glial cells | Ascl1, Lmx1a, and Nurr1 | Neuronal cells |
| 49. | Fibroblast-like cells | ASCL1 and PAX6 | Neuronal cells |
| | from retinal tissues | | |
| 50. | Pluripotent stem cell- | Brn2, Ascl1, Myt1l and Neurod | Neuronal cells |
| | derived cardiomyocytes | | |
| 51. | Fibroblasts | ASCL1, ISL1, NEUROD1, | Motor neurons |
| | | BRN2, HB9, LHX3, MYT1L, | |
| | | and NGN2 | |
| 52. | Fibroblasts | SOX2, GATA3, and | Neurocytes |
| | | NEUROD1 | |
| 53. | Dermal fibroblasts | SOX2 and PAX6 | Neural precursor cells |
| 54. | Embryonic fibroblasts | Ptf1a | Neural stem cells |
| | and Newborn foreskin | | |
| | fibroblasts | | |
| 55. | Adult fibroblasts | SOX2; SOX2 and PAX6; | Neural precursor cells |
| | | SOX2 and LMX1A; SOX2, | |
| | | LMX1A, and FOXA2 | |
| 56. | Spiral ganglion non- | Ascl1 and Neurod | Neuronal cells |
| | neuronal cells | | |
| 57. | Umbilical cord | SOX2, ASCL1, and | Neuronal cells |
| | mesenchymal stem | NEUROG2 | |
| | cells | | |
| 58. | Pericytes | ASCL1 and SOX2 | Neuronal cells |
| 59. | Cord blood CD133(+) | FOXM1, SOX2, MYC, SALL4, | Neuronal cells |
| | cells | and STAT6 | |
| 60. | Hepatocytes | Suz12, Ezh2, Meis1, Sry, | Neuronal cells |
| | | Smarca4, Esr1, Pparg, and | |
| | | Stat3 | |
| 61. | Peripheral CD34(+) | AR, SOX2, SMAD3, MYC, | Neural stem cells |
| | cells | JUN, WT1, TAL1, SPI1, and | |
| | | RUNX1 | |
| 62. | Urine epithelial-like cells | POU3F2, SOX2, BACH1, AR, | Neural stem cells |
| | | PBX1, and NANOG | |
| 63. | Muller glia cells | Bmi1, Spi1, Lmo2, and Cebpd | Neural stem cells |
| 64. | Astrocytes and Foreskin | Ascl1, Phox2b, Ap-2a, Gata3, | Noradrenergic |
| | fibroblasts | Hand2, Nurr1, and Phox2a | neurons |
| 65. | Bone marrow-derived | MSI1, NGN2, and MBD2 | Neural precursor cells |
| | cells, Fibroblasts, and | | |
| | Keratino-cytes | | |
| 66. | Microglial cells | Neurod 1 | Neuronal cells |
| 67. | Cardiac fibroblasts | Gata4, Mef2c, and Tbx5 | Cardiomyocytes |
| 68. | Cardiac fibroblasts | Gata4, Mef2c, Tbx5, and | Cardiomyocytes |
| | | Hand2 | |
| 69. | Cardiac fibroblasts and | Mef2c and Tbx5 + Myocd or | Cardiomyocytes |
| | Embryonic fibroblasts | Gata4 | |
| 70. | Cardiac fibroblasts | GATA4, MEF2C, TBX5, | Cardiomyocytes |
| | | MESP1, and MYOCD | |
| 71. | Embryonic stem cells- | GATA4, MEF2C, TBX5, | Cardiomyocytes |
| | derived fibroblasts | ESRRG, MESP1, ZFPM2, and | |
| | | MYOCD | |
| 72. | Adult fibroblasts | Gata4, Hand2, Mef2c, Tbx5, | Cardiomyocytes |
| | | and Znf281 | |
| 73. | Embryonic fibroblasts | Hnf4a and Foxa1, Foxa2, or | Hepatocytes |
| | and Adult dermal | Foxa3 | |
| | fibroblasts | | |
| 74. | Caudal fibroblasts | Gata4, Hnf1a, and Foxa3 + | Hepatocytes |
| | | p19^{Arf} knockdown | |
| 75. | Fetal and adult | FOXA3, HNF1A, and HNF4A + | Hepatocytes |
| | fibroblasts and Adipose | SV40 large T antigen | |
| | tissue-derived | | |
| | mesenchymal stem | | |
| | cells | | |
| 76. | Fibroblasts (BJ and | HNF1A and Any two of the | Hepatocytes |
| | MRC-5 cells) | three factors: FOXA1, FOXA3, | |
| | Hepatocytes cells) | and HNF4A | |
| 77. | Liver cells in mouse | Foxa3, Gata4, Hnf1a, and | Hepatocytes |
| | models of chronic liver | Hnf4a | |
| | disease | | |
| 78. | Fetal lung fibroblasts | ATF5, PROX1, FOXA2, | Hepatocytes |
| | | FOXA3, and HNF4A | |
| 79. | Fibroblasts | OCT4, FOXA2, HNF1A, and | Hepatocytes |
| | | GATA3 | |
| 80. | Embryonic fibroblasts | Foxa3, Hnf1a, and Gata4 | Hepatocytes |
| 81. | Embryonic fibroblasts | Hnf4a, Foxa3, Klf4, and c-Myc | Hepatocytes |
| 82. | Liver cells in vivo | Pdx1 | β cells |
| 83. | Pancreatic exocrine | Ngn3, Pdx1, and Mafa | β cells |
| | cells in vivo | | |
| 84. | Hepatocytes | Ngn3 | Islet cells |
| 85. | Liver cells | PDX1, PAX4, and MAFA | β cells |
| 86. | Cultured adult | Pdx1, Ngn3, and Mafa | β cells |
| | pancreatic duct cells | | |
| 87. | Gallbladder cells | Pdx1, Ngn, Mafa, and Pax6 | β cells |
| 88. | T precursor cells | Cebpa or Cebpb | Macrophages |
| 89. | T precursor cells | Pu.1 | Dendritic cells |
| 90. | B cells | Pax5 knockout | T cells |
| 91. | Fibroblasts (3T3 cells), | Pu.1 and Cebpa or Cebpb | Macrophage-like cells |
| | Embryonic fibroblasts, | | |
| | and Adult skin | | |
| | fibroblasts | | |
| 92. | B cells | Gata1, Scl, and Cebpa | Erythroid cells |
| 93. | Fibroblasts (3T3 cells) | Nfe2, Mafg, and Mafk | Megakaryocyte |
| | and Adult dermal | | |
| | fibroblasts | | |
| 94. | Skin fibroblasts | Spl1, Cebpa, Mnda, and Irf8 | Monocytes |
| 95. | Embryonic fibroblasts | Erg, Gata2, Lmo2, Runx1c, | Hematopoietic |
| | and Adult ear skin | and Scl | progenitor cells |
| | fibroblasts | | |
| 96. | Fibroblasts | Pu.1, Irf8, and Batf3 | Antigen-presenting |
| | | | dendritic cells |
| 97. | Neonatal foreskin | c-MYC, KLF4, and SOX9 | Chondrogenic cells |
| | fibroblasts | | |
| 98. | Dermal fibroblasts | OCT3/4 and OCT6 or OCT9 + | Osteoblasts |
| | | L-MYC, c-MYC, or N-MYC | |
| 99. | Fibroblasts | RUNX2, OCT4, OSTERIX, | Osteoblasts |
| | | and L-MYC | |
| 100. | Gingival fibroblasts and | OCT4, OSTERIX, and L-MYC | Osteoblasts |
| | Adult dermal fibroblasts | | |
| 101. | Embryonic fibroblasts | c-Myc, Oct4, and hLMP3 | Osteoblasts |
| 102. | Fibroblasts (C3H10T1/2 | Myod | Myoblasts |
| | cells) | | |
| 103. | Dermal fibroblasts | MYOD1 and MYCL | Myoblasts |
| 104. | Embryonic fibroblasts | Mef2b and Pitx1 + Pax3 or | Skeletal muscle |
| | | Pax7 | progenitor cells |
| 105. | Adult fibroblasts | Pax7, Mef2b, and Myod | Skeletal muscle |
| | | | progenitor cells |
| 106. | Embryonic fibroblasts | Prdm16 and Cebpb | Brown fat cells |
| | and Newborn foreskin | | |
| | fibroblasts | | |
| 107 | Embryonic fibroblasts | Nr5a1, Wt1, Dmrt1, Gata4, | Sertoli cells |
| | | and Sox9 | |
| 108. | Iris-derived cells | CRX, RAX, and | Photoreceptor cells |
| | | NEUROD | |
| 109. | Embryonic fibroblasts | Mitf, Sox10, and Pax3 | Melanocytes |
| | and Adult tail-tip dermal | | |
| | fibroblasts | | |
| 110. | Adipose tissue-derived | SOX18 | Endothelial cells |
| | stromal cells | | |
| 111. | Dermal fibroblasts | CRX, RAX, OTX2, and | Photoreceptor cells |
| | | NEUROD | |
| 112. | Embryonic fibroblasts | Foxn1 | Thymic epithelial cells |
| 113. | Fibroblasts | NF-κB and LEF-1 | Sweat gland cells |
| 114. | Amniotic fluid stem cells | OCT4 | Pluripotent stem cells |
| 115. | Cardiac mesenchymal | Klf4 and c-Myc | Adipocytes |
| | progenitors | | |
| 116 | Embryonic fibroblasts, | Emx2, Hnf1b, Hnf4a, and | Renal tubular |
| | Adult tail-tip dermal | Pax8 | epithelial cells |
| | fibroblasts, Postnatal | | |
| | foreskin fibroblasts, and | | |
| | Fetal dermal fibroblasts | | |
| 117. | Endothelial progenitor | MYOCD | Smooth muscle cells |
| | cells | | |
| 118 | Embryonic stem cells | Cdx2, Arid3a, and Gata3 | Trophoblast stem cells |
| 119. | Embryonic fibroblasts | Dmrt1, Gata4, and Nr5a1 | Leydig cells |
| | and Adult tail-tip dermal | | |
| | fibroblasts | | |
| 120 | Postnatal dermal | ER71/ETV2 (ETS | Endothelial cells |
| | fibroblasts | variant 2) | |
| 121. | Dermal fibroblasts | PPARG2 | Adipocytes |
| 122. | Embryonic fibroblasts | Hnf4a, Foxa3, Gata6, and | Intestine progenitor |
| | and Umbilical vein | Cdx2 | cells |
| | endothelial cells | | |
| 123. | Embryonic fibroblasts | Myocd, Gata6, and Mef2c | Smooth muscle cells |
| | and Adult dermal | | |
| | fibroblasts | | |
| 124. | Epidermal cells | Foxc1 | Sweat gland cells |
| 125 | Renal proximal tubular | SNAI2, EYA1, and SIX1 | Nephron progenitor |
| | epithelial (HK2) cells | | cells |
| 126. | Fibroblasts (BJ and | HNF1A and Any two of the | Hepatocytes |
| | MRC-5 cells) | three factors: FOXA1, FOXA3, | |
| | | and HNF4A | |
| 127. | Non-sensory cochlear | Ascl1; Ascl1 and Neurod | Neuronal cells |
| | epithelial cells | | |
| 128. | Cardiac fibroblasts | Gata4, Mef2c, and Tbx5 | Cardiomyocytes |
| 129. | Astrocytes | Sox2 | Neural stem cells |
| 130. | Fetal and embryonic | Ascl1, Brn2, and Myt1l | Neuronal cells |
| | fibroblasts and Brain | | |
| | cells in vivo | | |
| 131. | Peripheral blood mono- | CRX, RAX1, and NEUROD1 | Photoreceptor cells |
| | nuclear cells | | |
| 132. | Gingival fibroblasts and | OCT4, OSTERIX, and L-MYC | Osteoblasts |
| | Adult dermal fibroblasts | | |
| 133 | Fibroblasts | OCT4, FOXA2, HNF1A, and | Hepatocytes |
| | | GATA3 | |
| 134. | Fibroblasts | SOX2, GATA3, and | Neurocytes |
| | | NEUROD1 | |
| 135. | Fibroblasts (3T6 cells) | Ascl1, Brn2, and Foxa1 | Neuronal cells |
| 136. | Mesenchymal stem | Hnf4a and Foxa3 | Hepatocytes |
| | cells | | |
| 137. | Dermal fibroblasts | ETV2 | Endothelial pro-genitor |
| | | | cells |
| 138. | Fibroblasts (3T6 cells) | Ascl1, Brn4, and Tcf3 | Neuronal cells |
| 139. | Mesenchymal stem | SOX2 | Neural stem cells |
| | cells and Dermal | | |
| | fibroblasts | | |
| 140. | Adult fibroblasts | SOX2; SOX2 and PAX6; | Neural precursor cells |
| | | SOX2 and LMX1A; SOX2, | |
| | | LMX1A, and FOXA2 | |
| 141 | Dermal fibroblasts | SOX2 and PAX6 | Neural precursor cells |
| 142. | Embryonic fibroblasts | Hnf4a and Foxa3 | Hepatocytes |
| 143. | Foreskin fibroblasts | ASCL1 + miR-124 + P53 | Neuronal cells |
| | | knock-down | |
| 144 | Bone marrow-derived | MSI1, NGN2, and MBD2 | Neural precursor cells |
| | cells, Fibroblasts, and | | |
| | Keratinocytes | | |
| 145 | Renal proximal tubular | SNAI2, EYA1, and SIX1 | Nephron progenitor |
| | epithelial (HK2) cells | | cells |
| 146. | Cardiac fibroblasts | miR-1, miR-133, miR-208, and | Cardiomyocytes |
| | | miR-499 | |
| 147. | Cardiac fibroblasts and | Gata4, Mef2c, and Tbx5 + | Cardiomyocytes |
| | Embryonic fibroblasts | miR-133; Gata4, Mef2c, Tbx5, | |
| | | Mesp1, and Myocd + miR-133 | |
| 148. | Fibroblasts | GATA4, MEF2C, TBX5, | Cardiomyocytes |
| | | ESRRG, MESP1, | |
| | | MYOCARDIN, ZFPM2, and | |
| | | HAND2 + miR-1 | |
| 149 | Adult fibroblasts | miR-9/9* and miR-124 | Neuronal cells |
| 150. | Adult fibroblasts | ISL1 and LHX3 + miR-9/9* and | Spinal cord motor |
| | | miR-124 | neurons |
| 151. | Brain vascular pericytes | ASCL1, MYT1L, BRN2, and | Cholinergic neuronal |
| | | TLX3 + miR-124 | cells |

The contacting may be performed by any of the methods described above (e.g., by transfection, electroporation, and/or the use of circRNA-LNP complexes).

In some embodiments, the cells are contacted with the circular RNA once. In some embodiments the cells are contacted with the circular RNA more than once, e.g., 2, 3, 4, 5, 6, 7, 8, 9, or 10 times. In some embodiments, the contacting is performed at effective intervals. The effective intervals may be, for example, once per day, once every other day, once every three days, once per week, once every two weeks, or once per month.

As explained above, the methods of directly converting a cell from a first cell type to a second cell type may comprise maintaining the cell under conditions under which the cell is converted to the second cell type. Such conditions are known to those of skill in the art and may vary by cell type. As one example, after the cells have been contacted with one or more circular RNAs they can be cultured in standard media which is optionally supplemented with various reprogramming factors. The cells will be monitored to observe morphology, and the presence of markers characteristic of the second cell type.

Also provided herein are transdifferentiated cells produced using the methods described herein.

Also provided herein are compositions comprising a transdifferentiated cell, wherein the transdifferentiated cell comprises one or more circular RNAs encoding a transdifferentiation factor. In some embodiments, the transdifferentiation factor is any one of the transdifferentiation factors or combinations of transdifferentiation factors listed in Table 5. In some embodiments, the transdifferentiated cell is any one of the second cell types listed in Table 5. In some embodiments, the transdifferentiated cell is derived from a first cell type that is any one of the first cell types listed in Table 5.

Differentiation of iPSCs Using Circular RNAs

Also provided is an iPSC produced using the methods described herein. In some embodiments, the iPSC expresses one or more of Oct4, SOX2, Lin 28, SSEA4, SSEA3, TRA-1-81, TRA-1-60, CD9, Nanog, FbxI5, EcatI, EsgI, Eras, Gdf3, Fgf4, Cripto, DaxI, Zpf296, Slc2a3, RexI, UtfI, and Nat1.

Also provided herein is a differentiated cell derived from an iPSC produced using the methods described herein. Methods for differentiating an iPSC are known to those of skill in the art. In some embodiments, the differentiated cell is a muscle cell, a neural cell, a cell of the central nervous system, an ocular cell, a chondrocyte, an osteocyte, a tendon cell, a cardiomyocyte, a hepatocyte, a kidney cell, an islet cell, a keratinocyte, a T-cell, or a NK-cell. In some embodiments, the differentiated cell is a cell type belonging but not limited to, for example—the muscle, neural, ocular, cartilage, bone, connective tissue, heart, liver, kidney, pancreas, skin, or hematopoietic lineages.

In some embodiments, an iPSC described herein (or an iPSC produced using a method that is not described herein) may be differentiated by contacting the iPSC with one or more circular RNAs encoding a differentiation factor. For example, in some embodiments, an iPSC is contacted with a circular RNA, or a DNA molecule encoding the same, which encodes a differentiation factor capable of differentiating the iPSC into a cell type of interest, such as a T-cell. In some embodiments, the differentiation factor is selected from RORA, HLF, MYB, KLF4, ERG, SOX4, LUC, HOXA9, HOXA10, and HOXA5. In some embodiments, the iPSC is contacted with at least one, at least two, at least three, at least four, at least five, at least six, at least seven, at least eight, at least nine, at least ten, or at least eleven circular RNAs, wherein each circular RNA encodes a differentiation factor selected from RORA, HLF, MYB, KLF4, ERG, SOX4, LUC, HOXA9, HOXA10, and HOXA5. In some embodiments, an iPSC is contacted with at least one, at least two, at least three, at least four, or at least five circular RNAs, wherein each circular RNA encodes a differentiation factor selected from HOXA9, ERG, RORA, SOX4, or MYB. In some embodiments, the iPSC is contacted with a plurality of circular RNAs, wherein each circular RNA encodes at least one of HOXA9, ERG, RORA, SOX4, or MYB. In some embodiments, the iPSC is contacted with at least one circular RNA, wherein the circRNA encodes one or more of the differentiation factors listed in Table 6. In some embodiments, the iPSC is additionally contacted with an EZH1 shRNA. The EZH1 shRNA expression may facilitate a switch from lineage restricted hematopoietic progenitors to progenitors with multi-lymphoid potential.

In some embodiments, an iPSC is differentiated into a CD34+CD38− cell. In some embodiments, contacting the iPSC with one or more of the circular RNAs encoding one or more of the following differentiation factors differentiates the iPSC into a CD34+CD38− cell: RORA, HLF, MYB, KLF4, ERG, SOX4, LUC, HOXA9, HOXA10, or HOXA5.

In some embodiments a CD34+CD45+ myeloid precursor cell is contacted with a circular RNA, or a DNA molecule encoding the same, that encodes one or more of RORA, HLF, MYB, KLF4, ERG, SOX4, LUC, HOXA9, HOXA10, or HOXA5. In some embodiments a CD34+CD45+ myeloid precursor cell is contacted with a circular RNA, or a DNA molecule encoding the same, that encodes one or more of HOXA9, ERG, RORA, SOX4, or MYB. In some embodiments, contacting with one or more circular RNAs iPSC as described above transdifferentiates the CD34+CD45+ cell into a CD34+CD38− cell. In some embodiments, the cells resulting after the contacting are self-renewing HSPCs (hematopoetic stem and progenitor cells) with erythroid and lymphoid potential.

In some embodiments, the iPSC produced using the methods described herein is younger as compared to an iPSC produced using traditional methods, such as use of a viral vector encoding a reprogramming factor or transfection of a linear RNA encoding a reprogramming factor. As described herein, “younger” refers to the fact that the cell is reprogrammed faster (i.e., within about 5, about 6, about 7, or about 8 days after transfection) as compared to traditional methods (i.e., about 9 days or more).

In some embodiments, the iPSC expresses different levels of one or more biomarkers as compared to an iPSC produced using traditional methods. For example, in some embodiments, the iPSC expresses lower levels of markers associated with cellular stress and/or cell death (apoptosis), as compared to an iPSC produced using traditional methods. For example, in some embodiments, the iPSC expresses lower levels of one or more heat shock proteins or caspases.

In some embodiments, the genome of the iPSC has different epigenetic modifications as compared to an iPSC produced using traditional methods. For example, in some embodiments, the iPSC may comprise altered levels of DNA methylations and/or histone modifications.

In some embodiments, a T-cell is contacted with one or more circular RNAs (or DNA molecules encoding the same) which encode factors that can improve the efficacy of the T-cell. In this context, improving the efficacy refers to promoting survival of the T-cell, and/or its anti-tumor activity when used in an immune-oncology setting. For example, the T-cell may be contacted with one or more circular RNAs that encode IL-12, IL-18, IL-15, or IL-7.

In some embodiments, a T-cell is contacted with one or more circular RNAs (or DNA molecules encoding the same) which improve the ability of the T-cell to home to a tumor tissue. For example, the T-cell may be contacted with one or more circular RNAs that encode CXCR2, CCR2B, or heparanase.

In some embodiments, a T-cell is contacted with one or more circular RNAs (or DNA molecules encoding the same) which help improve survival and/or promote the switch to a central memory phenotype. For example, the T-cell may be contacted with one or more circular RNAs that encode Suv39h1.

### Combination Methods for Reprogramming and Editing the Genome of a Cell

By combining methods for generating iPSCs with methods for genome editing thereof, the diagnostic and therapeutic power of iPSCs is enhanced. As used herein, the terms “genome editing” and “editing the genome” refer to modification of a specific locus of a nucleic acid (e.g., a DNA or an RNA) of a cell. Genome editing can correct pathology-causing genetic mutations derived from diseased patients and similarly can be used to induce specific mutations in disease-free wild-type cells (such as iPSCs). Accordingly, the instant disclosure provides combination methods for reprogramming and editing the genome of a cell. In some embodiments, the circular RNAs described herein may be used in methods for reprogramming and editing the genome of a cell.

Genome editing may comprise, for example, inducing a double stranded DNA break in the region of gene modification. In some embodiments, a locus of the DNA is replaced with an exogenous sequence by supplementation with a targeting vector. Any one of the following enzymes may be used to edit the DNA of a cell: a zinc-finger nuclease, a homing endonuclease, a TALEN (transcription activator-like effector nuclease), a NgAgo (argonaute endonuclease), a SGN (structure-guided endonuclease), a RGN (RNA-guided nuclease), or modified or truncated variants thereof. In some embodiments, the RNA-guided nuclease is an RNA-guided nuclease disclosed in any one of WO 2019/236566 (e.g., APG05083.1, APG07433.1, APG07513.1, APG08290.1, APG05459.1, APG04583.1, and APG1688.1 RNA-guided nucleases), WO 2021/030344 (e.g., APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, and APG07433.1 RNA-guided nucleases), and WO 2020/139783 (APG00969, APG03128, APG09748, APG00771, APG02789, APG09106, APG02312, APG07386, APG09980, APG05840, APG05241, APG07280, APG09866, APG00868 RNA-guided nucleases), each of which is incorporated herein by reference in its entirety. In some embodiments, the RNA-guided nuclease is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or modified or truncated variants thereof.

In some embodiments, a Cas9 nuclease is used to edit the genome of a cell. Cas9 is a large multifunctional protein having two putative nuclease domains, the HNH and RuvC-like. The HNH and the RuvC-like domains cleave the complementary 20-nucleotide sequence of the crRNA and the DNA strand opposite the complementary strand respectively. Several variants of the CRISPR-Cas9 system exists, and any one of these variants may be used in the methods disclosed herein: (1) The original CRISPR-Cas9 system functions by inducing DNA double-stranded breaks which are triggered by the wild-type Cas9 nuclease directed by a single RNA. (2) The nickase variant of Cas9(D10A mutant) which is generated by the mutation of either the Cas9 HNH or the RuvC-like domain is directed by paired guide RNAs. (3) Engineered nuclease variant of Cas9 with enhanced specificity (eSpCas9). (4) Catalytically dead Cas9 (dCas9) variant is generated by mutating both domains (HNH and RUvC-like). dCas9, when merged with a transcriptional suppressor or activator can be used to modify transcription of endogenous genes (CRISPRa or CRISPRi) or when fused with fluorescent protein can be used to image genomic loci. (5) CRISPR-Cas9 fused with cytidine deaminase, results in a variant which induces the direct conversion of cytidine to uridine, hence circumventing the DNA double-stranded break. In some embodiments, the Cas9 nuclease is isolated or derived from S. pyogenes or S. aureus.

Cas9, and other RNA-guided nucleases, require a RNA guide sequence (“guide RNA” or “gRNA”) to target a specific locus. In some embodiments, the gRNA is a single-guide (“sgRNA”). The sgRNA may comprise a spacer sequence and a scaffold sequence. The spacer sequence is complementary to the target cleavage sequence, and directs the enzyme thereto. The scaffold region binds to the RNA-guided nuclease enzyme.

Exemplary enzymes which may be used to edit the RNA of a cell include, but are not limited to, enzymes of the ADAR (adenosine deaminase acting on RNA) family. For example, the enzyme may be human ADAR1, ADAR2, or ADAR3, or a modified or truncated variant thereof. In some embodiments, the enzyme may be an ADAR from squid (e.g., Loligo pealeii) such as sqADAR2, or a modified or truncated variant thereof. In some embodiments, the enzyme may be an ADAR from C. elegans (e.g., ceADAR1 or ceADAR2) or D. melanogaster (e.g., dADAR), or a modified or truncated variant thereof.

In some embodiments, a method for reprogramming and editing the genome of a cell comprises (i) contacting a cell with a circular RNA comprising a protein-coding sequence, wherein the protein-coding sequence encodes at least one reprogramming factor, and (ii) contacting the cell with an enzyme capable of editing the DNA or RNA of the cell.

In some embodiments, a method for reprogramming and editing the genome of a cell comprises contacting a cell with (i) a circular RNA comprising a protein-coding sequence, wherein the protein-coding sequence encodes at least one reprogramming factor, and (ii) a nucleic acid encoding an enzyme capable of editing the DNA or RNA of the cell.

In some embodiments, cell is contacted with the circular RNA before it is contacted with the enzyme or the nucleic acid encoding the same. In some embodiments, the cell is contacted with the circular RNA after it is contacted with the enzyme or the nucleic acid encoding the same. In some embodiments, the cell is contacted with the circular RNA at approximately the same time that it is contacted with the enzyme or the nucleic acid encoding the same.

In some embodiments, the methods for reprogramming and editing the genome of a cell further comprise contacting the cell with a nucleic acid encoding a guide RNA, or a guide RNA.

A composition for reprogramming and editing the genome of a cell may comprise, for example, a circular RNA (or a DNA molecule encoding the same) and an enzyme capable of editing DNA or RNA (or a DNA or RNA molecule encoding the same). In some embodiments, the circular RNA comprises a protein-coding sequence. In some embodiments, the circular RNA does not encode a protein. In some embodiments, the circular RNA is circBIRC6 (SEQ ID NO: 29), circCORO1C (SEQ ID NO: 30), or circMAN1A2 (SEQ ID NO: 31).

### Combination Methods for Transdifferentiating and Editing the Genome of a Cell

The circular RNAs described herein may be also used in methods for transdifferentiating and editing the genome of a cell. Accordingly, provided herein are compositions and methods for transdifferentiating and editing the genome of a cell.

In some embodiments, a method for transdifferentiating and editing the genome of a cell comprises contacting a cell with (i) a circular RNA comprising a protein-coding sequence, wherein the protein-coding sequence encodes at least one transdifferentiation factor, and (ii) an enzyme capable of editing the DNA or RNA of the cell. In some embodiments, the transdifferentiation factor is selected from any of those listed in Table 5.

In some embodiments, a method for transdifferentiating and editing the genome of a cell comprises contacting a cell with (i) a circular RNA comprising a protein-coding sequence, wherein the protein-coding sequence encodes at least one transdifferentiation factor, and (ii) a nucleic acid encoding an enzyme capable of editing the DNA or RNA of the cell

The enzymes used to edit DNA or RNA in a method of transdifferentiating and editing the genome of a cell may be any of the enzymes listed above.

In some embodiments, cell is contacted with the circular RNA before it is contacted with the enzyme or the nucleic acid encoding the same. In some embodiments, the cell is contacted with the circular RNA after it is contacted with the enzyme or the nucleic acid encoding the same. In some embodiments, the cell is contacted with the circular RNA at approximately the same time that it is contacted with the enzyme or the nucleic acid encoding the same.

In some embodiments, the methods for transdifferentiating and editing the genome of a cell further comprise contacting the cell with a nucleic acid encoding a guide RNA, or a guide RNA.

A composition for transdifferentiating and editing the genome of a cell may comprise, for example, a circular RNA (or a DNA molecule encoding the same) and an enzyme capable of editing DNA or RNA (or a DNA or RNA molecule encoding the same). In some embodiments, the circular RNA comprises a protein-coding sequence. In some embodiments, the circular RNA does not encode a protein. In some embodiments, the circular RNA is circBIRC6 (SEQ ID NO: 29), circCORO1C (SEQ ID NO: 30), or circMAN1A2 (SEQ ID NO: 31). In some embodiments, the circular RNA encodes a reprogramming factor disclosed herein. In some embodiments, the circular RNA encodes one or more Oct3/4, Klf4, Sox2, Nanog, Lin28 (e.g., Lin28A or Lin28B), c-Myc, and L-Myc. In some embodiments, the circular RNA encodes one or more of the transdifferentiation factors listed in Table 5.

### Additional Methods

As will be understood by those of skill in the art, the circular RNAs described herein, and related compositions, may be useful for one or more of the following methods.

In some embodiments, provided herein is a method reprogramming a cell which produces reduced cell death as compared to a method using linear RNA, the method comprising contacting a cell with a circular RNA, a complex, a vector, or a composition as described herein, and maintaining the cell under conditions under which the protein is expressed. In some embodiments, the reprogramming-induced cell death is reduced by at least 10%, at least 20%, at least 30%, at least 40%, at least 50%, at least 60%, at least 70%, at least 80%, at least 90%, at least 100%, at least 200%, at least 500% or more relative to a reprogramming method using linear RNA. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of reducing time from reprogramming to picking, the method comprising contacting a cell with a circular RNA, a complex, a vector or a composition described herein, and maintaining the cell under conditions under which the protein is expressed, wherein the time from reprogramming to picking is reduced relative to a reprogramming method using linear RNA. As used herein, the term “picking” refers to manual selection if iPSC colonies by mechanical dissociation. In some embodiments, the time is reduced by at least 10%, at least 20%, at least 30%, at least 40%, at least 50%, at least 60%, at least 70%, at least 80%, at least 90%, at least 100%, at least 200%, at least 500% or more relative to a reprogramming method using linear RNA. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of reducing the number of transfections induce to effect reprogramming of a cell, the method comprising contacting a cell with a circular RNA, a complex, a vector, or a composition described herein, and maintaining the cell under conditions under which the protein is expressed. In some embodiments, the number of transfections is reduced relative to a method using linear RNA. In some embodiments, the number of transfections to induce reprogramming of the cell is 1, 2, 3, 4, 5, 6, or 7. In some embodiments, the number of transfections is reduced by at least 10%, at least 20%, at least 30%, at least 40%, at least 50%, at least 60%, at least 70%, at least 80%, at least 90%, at least 100%, at least 200%, at least 500% or more relative to a method using linear RNA. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28, and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is method of increasing duration of protein expression in a cell, the method comprising contacting a cell with a circular RNA, a complex, a vector, or a composition described herein, and maintaining the cell under conditions under which the protein is expressed. In some embodiments, the duration of protein expression is increased relative to a method comprising transfection of the cell with a linear RNA encoding the same protein. In some embodiments, the duration of protein expression is increased by at least 10%, at least 20%, at least 30%, at least 40%, at least 50%, at least 60%, at least 70%, at least 80%, at least 90%, at least 100%, at least 200%, at least 500% or more relative to a method comprising transfection of the cell with a linear RNA encoding the same protein. In some embodiments, the duration of protein expression is increased by at least 1 hour, at least 4 hours, at least 8 hours, at least 12 hours, at least 1 day, at least 2 days, at least 3 days, at least 4 days, at least 5 days, at least 6 days, at least 1 week, at least 2 weeks, at least 3 weeks, or longer relative to a method comprising transfection of the cell with a linear RNA encoding the same protein. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of improving cellular reprogramming efficiency, the method comprising contacting a cell with circular RNA, a complex, a vector, or a composition described herein, and maintaining the cell under conditions under which the protein is expressed, wherein the efficacy of cellular reprogramming is increased relative to a cellular reprogramming method in which linear RNA is used. In some embodiments, cellular reprogramming efficiency is increased by at least 10%, at least 20%, at least 30%, at least 40%, at least 50%, at least 60%, at least 70%, at least 80%, at least 90%, at least 100%, at least 200%, at least 500% or more relative to a method in which linear RNA is used. Enhanced cellular reprogramming efficiency may be observed based on qualitative and/or qualitative assessments including, but not limited to, reduced cell death, reduced immune response induced stress as measured by IFN-gamma secretion, reduced stress response gene induction. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of increasing the number of reprogrammed cell colonies formed after reprogramming, the method comprising contacting a cell with circular RNA, a complex, a vector, or a composition, and maintaining the cell under conditions under which the protein is expressed, wherein the number of reprogrammed cell colonies formed after reprogramming is increased relative to a cellular reprogramming method in which linear RNA is used. In some embodiments, the number of reprogrammed cell colonies is increased by at least 10%, at least 20%, at least 30%, at least 40%, at least 50%, at least 60%, at least 70%, at least 80%, at least 90%, at least 100%, at least 200%, at least 500% or more relative to a method in which linear RNA is used. In some embodiments, the increased number of colonies may be observed about 7, about 8, about 9, about 10, about 11, about 12, about 13, about 14, about 15, about 16, about 17, about 18, about 19, or about 20 days post-transfection with one or more circRNAs encoding a transcription factor. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of reprogramming cells in suspension, the method comprising contacting a cell in suspension with a circular RNA, a complex, a vector, or a composition described herein, and maintaining the cell under conditions under which the protein is expressed. In some embodiments, the cells express CD34 (i.e., they are CD34+). In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B) (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a method of improving morphological maturation of reprogrammed colonies, the method comprising contacting a cell in suspension with a circular RNA, a complex, a vector, or a composition described herein, and maintaining the cell under conditions under which the protein is expressed, wherein the morphological maturation is improved relative to a cellular reprogramming method in which linear RNA is used. Improved morphological maturation may include, for example, more tightly-packed colonies, colonies where more cells have a uniform shape and diameter, colonies comprising a clearly-defined border, and cells within iPSC colonies comprising a higher nuclear to cytoplasmic ratio and/or prominent nucleoli. In some embodiments, the morphological maturation of the reprogrammed colonies is improved by at least 10%, at least 20%, at least 30%, at least 40%, at least 50%, at least 60%, at least 70%, at least 80%, at least 90%, at least 100%, at least 200%, at least 500% or more relative to a method in which linear RNA is used. In some embodiments, the cell is contacted with a combination of circular RNAs, wherein the combination of circular RNAs is selected from: (i) circOct3/4, circKlf4, circSox2, circNanog, circLin28 (e.g., Lin28A or Lin28B), and circ c-Myc; (ii) circOct3/4, circKlf4, circSox2, circNanog, and circLin28 (e.g., Lin28A or Lin28B); (iii) circOct3/4, circKlf4, circSox2, circNanog, circLin28 (e.g., Lin28A or Lin28B), and circL-Myc; (iv) circOct3/4, circKlf4, circSox2, circNanog, and circLin28 (e.g., Lin28A or Lin28B); (v) circOct3/4, circKlf4, circSox2, and circC-Myc; (vi) circOct3/4, circKlf4, circSox2, and circL-Myc; or (vii) circOct3/4, circKlf4, and circSox2. In some embodiments, the cell is contacted with circMyoD.

Also provided herein is a suspension culture comprising one or more CD34-expressing cells, wherein the CD34-expressing cells comprise one or more circRNAs encoding a reprogramming factor. In some embodiments, the reprogramming factor is selected from Oct3/4, Klf4, Sox2, Nanog, Lin28 (e.g., Lin28A or Lin28B), c-Myc, and L-Myc.

Also provided herein is a method for inducing a mesenchymal-to-epithelial transition (MET) of a somatic cell to an iPSC comprising contacting the somatic cell with one or more circular RNA encoding a reprogramming factor.

Also provided herein is a method for inducing a mesenchymal-to-epithelial transition (MET) of a somatic cell to an iPSC comprising contacting the somatic cell with one or more circular RNA encoding a reprogramming factor.

### Compositions, and Cells

Also provided herein are compositions comprising a circular RNA. In some embodiments, a composition comprises (i) a circular RNA and (ii) a carrier. In some embodiments, a composition comprises (i) a vector encoding a circular RNA and (ii) a carrier. Suitable carriers include, for example, sterile water, sterile buffer solutions (e.g., solutions buffered with phosphate, citrate or acetate, etc.), sterile media, polyalkylene glycols, hydrogenated naphthalenes (e.g., biocompatible lactide polymers), lactide/glycolide copolymer or polyoxyethylene/polyoxypropylene copolymers. In some embodiments, the carrier may comprise lactose, mannitol, substances for covalent attachment of polymers such as polyethylene glycol, complexation with metal ions or inclusion of materials in or on particular preparations of polymer compounds such as polylactate, polyglycolic acid, hydrogel or on liposomes, microemulsions, micelles, unilamellar or multilamellar vesicles, erythrocyte fragments or spheroplasts. In some embodiments, the pH of the carrier is in the range of 5.0 to 8.0, such as in the range of about 6.0 to about 7.0. In some embodiments, the carrier comprises salt components (e.g., sodium chloride, potassium chloride), or other components which render the solution, for example, isotonic. Further, the carrier may comprise additional components such as fetal calf serum, growth factors, human serum albumin (HSA), polysorbate 80, sugars or amino acids. Suitable vectors include plasmids (e.g., DNA plasmids) and viral vectors.

Also provided herein are cells comprising a circular RNA or a composition as described herein. In some embodiments, the cell is a prokaryotic cell. In some embodiments, the cell is a eukaryotic cell. In some embodiments, the cell is a mammalian cell (e.g., a murine, bovine, simian, porcine, equine, ovine, or human cell). In some embodiments, the cell is a human cell.

### Kits

Kits for reprogramming a cell and kits for producing iPSCs are also provided. In some embodiments, the kit comprises at least one circular RNA as described herein. In some embodiments, the kit comprises a vessel containing a circular RNA. In some embodiments, the kit comprises a plurality of vessels, wherein each vessel comprises a circular RNA. In some embodiments, a kit comprises a vessel comprising a plurality of circular RNA molecules, wherein each circular RNA molecule encodes a protein. In some embodiments, the kit also comprises a set of instructions for using the at least one circular RNA for reprogramming a cell.

In some embodiments, a kit comprises one or more circular RNAs wherein each circular RNA encodes at least one protein. In some embodiments, the kit may further comprise a linear or circular RNA that encodes a miRNA. In some embodiments, the kit may comprise a single vessel containing each of: (i) the one or more circular RNAs wherein each circular RNA encodes a protein, (ii) optionally, a circular RNA that does not encode any protein or miRNA (e.g., circBIRC6, circCORO1c, circMAN1A2), (iii) optionally, a linear or circular RNA that encodes a miRNA. In some embodiments, the kit may comprise a plurality of vessels, wherein each vessel comprises one of: (i) at least one circular RNA that encodes a protein, (ii) optionally, a circular RNA, or DNA molecule encoding the same, that does not encode any protein or miRNA, (iii) optionally, a linear or circular RNA that encodes a miRNA. In some embodiments, the kit also comprises a set of instructions for using the at least one circular RNA for reprogramming a cell.

In some embodiments, a kit for reprogramming somatic cells and/or generating iPSCs is provided. In some embodiments, the kit comprises at least one circular RNA encoding a reprogramming factor (e.g., a transcription factor). In some embodiments, the kit comprises a vessel containing a circular RNA. In some embodiments, the kit comprises a plurality of vessels, wherein each vessel comprises a circular RNA. In some embodiments, a kit comprises a vessel comprising a plurality of circular RNA molecules, wherein each circular RNA molecule encodes a transcription factor. In some embodiments, the kit also comprises a set of instructions for using the at least one circular RNA for reprogramming somatic cells and/or generating iPSCs.

In some embodiments, a kit comprises one or more circular RNAs wherein each circular RNA encodes at least one reprogramming factor. The reprogramming factors may be, for example, any one of the reprogramming factors listed in Table 1. In some embodiments, the kit may further comprise a circular RNA that does not encode any protein or miRNA, or a DNA molecule encoding the same. In some embodiments, the kit may further comprise a linear or circular RNA that encodes a miRNA, or a DNA molecule encoding the same. In some embodiments, the kit may comprise a single vessel containing each of: (i) the one or more circular RNAs wherein each circular RNA encodes a reprogramming factor, (ii) optionally, a circular RNA that does not encode any protein or miRNA (e.g., circBIRC6, circCORO1c, circMAN1A2), (iii) optionally, a linear or circular RNA that encodes a miRNA. In some embodiments, the kit may comprise a plurality of vessels, wherein each vessel comprises one of: (i) at least one circular RNA that encodes a reprogramming factor, (ii) optionally, a circular RNA that does not encode any protein or miRNA (e.g., circBIRC6, circCORO1c, circMAN1A2), (iii) optionally, a linear or circular RNA that encodes a miRNA. In some embodiments, the kit also comprises a set of instructions for using the at least one circular RNA for reprogramming a cell and/or generating an iPSC.

In some embodiments, a kit for transdifferentiating cells is provided. In some embodiments, the kit comprises at least one circular RNA encoding a reprogramming factor (e.g., a transcription factor). In some embodiments, the kit comprises a vessel containing a circular RNA. In some embodiments, the kit comprises a plurality of vessels, wherein each vessel comprises a circular RNA. In some embodiments, a kit comprises a vessel comprising a plurality of circular RNA molecules, wherein each circular RNA molecule comprises a sequence encoding a transdifferentiation factor. In some embodiments, the kit also comprises a set of instructions for using the at least one circular RNA for transdifferentiating cells.

In some embodiments, a kit comprises one or more circular RNAs wherein each circular RNA comprises a sequence that encodes at least one transdifferentiation factor. The transdifferentiation factors may be, for example, any one of the transdifferentiation factors listed in Table 5. In some embodiments, the kit may further comprise a circular RNA that does not encode any protein or miRNA. In some embodiments, the kit may further comprise a circular RNA that encodes a miRNA. In some embodiments, the kit may comprise a single vessel containing each of: (i) the one or more circular RNAs wherein each circular RNA encodes a transdifferentiation factor, (ii) optionally, a circular RNA that does not encode any protein or miRNA, (iii) optionally, a circular RNA that encodes a miRNA. In some embodiments, the kit may comprise a plurality of vessels, wherein each vessel comprises one of: (i) at least one circular RNA that encodes a transdifferentiation factor, (ii) optionally, a circular RNA that does not encode any protein or miRNA, (iii) optionally, a circular RNA that encodes a miRNA. In some embodiments, the kit also comprises a set of instructions for using the at least one circular RNA for expressing a transdifferentiation factor in a cell.

In some embodiments, a kit comprises a plurality of circular RNAs wherein each circular RNA encodes a reprogramming factor selected from Oct3/4, Klf4, Sox2, Nanog, Lin28 (e.g., Lin28A or Lin28B), c-Myc, and L-Myc, or a combination thereof. Each of the circular RNAs may be provided in separate vessels or may be provided in a single vessel.

In some embodiments, a kit comprises a plurality of circular RNAs wherein each of circular RNA encodes a reprogramming factor selected from Oct3/4, Sox2, and Klf4, or a combination thereof. Each of the circular RNAs may be provided in separate vessels or may be provided in a single vessel.

In some embodiments, a kit comprises a plurality of circular RNAs wherein each of circular RNA encodes a reprogramming factor selected from Oct3/4, Sox2, c-Myc, and Klf4, or a combination thereof. Each of the circular RNAs may be provided in separate vessels or may be provided in a single vessel.

In some embodiments, a kit comprises a plurality of circular RNAs, wherein each of circular RNA encodes a reprogramming factor selected from Oct3/4, Sox2, L-Myc, and Klf4, or a combination thereof. Each of the circular RNAs may be provided in separate vessels or may be provided in a single vessel.

In some embodiments, a kit comprises a plurality of circular RNAs wherein each circular RNA encodes a reprogramming factor selected from Oct3/4, Klf4, Sox2, Nanog, and Lin28 (e.g., Lin28A or Lin28B), or a combination thereof. Each of the circular RNAs may be provided in separate vessels or may be provided in a single vessel.

In some embodiments, a kit comprises a plurality of circular RNAs wherein each circular RNA encodes a reprogramming factor selected from Oct3/4, Klf4, Sox2, Nanog, Lin28 (e.g., Lin28A or Lin28B), and c-Myc, or a combination thereof. Each of the circular RNAs may be provided in separate vessels or may be provided in a single vessel.

In some embodiments, a kit comprises a plurality of circular RNAs wherein each circular RNA encodes a reprogramming factor selected from Oct3/4, Klf4, Sox2, Nanog, Lin28 (e.g., Lin28A or Lin28B), and L-Myc, or a combination thereof. Each of the circular RNAs may be provided in separate vessels or may be provided in a single vessel.

In some embodiments, a kit may comprise a linear RNA cable of being circularized, or a DNA sequence encoding the same. In some embodiments, a kit may further comprise one or more reagents for circularizing a linear RNA, such as an RNA or DNA ligase, or Mg2+ and guanosine 5′ triphosphate (GTP).

In some embodiments, a kit comprises: (i) a vessel comprising a circular RNA encoding OCT4 and a buffer (e.g., 1-10 mM sodium citrate, pH 6.5); (ii) a vessel comprising a circular RNA encoding SOX2 and a buffer (e.g., 1-10 mM sodium citrate, pH 6.5); (iii) a vessel comprising a cirRNA encoding KLF4 and a buffer (e.g., 1-10 mM sodium citrate, pH 6.5); and (iv) packaging and instructions therefor. The kit may further comprise a vessel comprising a circular RNA encoding c-MYC or L-MYC and a buffer (e.g., 1-10 mM sodium citrate, pH 6.5); a vessel comprising a cirRNA encoding LIN28 (e.g., Lin28A or Lin28B) and a buffer (e.g., 1-10 mM sodium citrate, pH 6.5); a vessel comprising a cirRNA encoding NANOG and a buffer (e.g., 1-10 mM sodium citrate, pH 6.5); or a combination thereof.

In some embodiments, a kit comprises: (i): (a) the circular RNA reprogramming factor(s) of any one or more circularized reprogramming factor combinations listed in Table 3, wherein each factor is contained individually in a separate vessel or wherein two or more of such factors are combined together in a single or plurality of vessels; and (ii) packaging and instructions therefor.

In some embodiments, a kit comprises: (i): the circular RNA reprogramming factor(s) of any one or more circularized reprogramming factor combinations listed in Table 3, wherein each factor is contained individually in a separate vessel or wherein two or more of such factors are combined together in a single or plurality of vessels; and wherein the circularized reprogramming factors and/or the circular RNAs of Table 3, respectively, are suspended in a buffer; and (iii) packaging and instructions therefor.

In any of the kits described above, the circular RNA may be provided in a composition that further comprises a buffer. The buffer may comprise, for example 1-10 mM sodium citrate. In some embodiments, the pH of the buffer is in the range of about 2 to about 12, such as about 6.5.

Sequences of the Disclosure

| Description | SEQUENCE | SEQ ID: |
|---|---|---|
| 2A_peptide_1 | EGRGSLLTCGDVEENPGP | 1 |
| 2A_peptide_2 | ATNFSLLKQAGDVEENPGP | 2 |
| 2A_peptide_3 | QCTNYALLKLAGDVESNPGP | 3 |
| 2A_peptide_4 | VKQTLNFDLLKLAGDVESNPGP | 4 |
| Kozak seq 1- | RCC (AUG) G | 5 |
| with the start | | |
| codon in | | |
| parentheses, | | |
| and the “R” at | | |
| position-3 | | |
| representing | | |
| a purine (A or | | |
| G) | | |
| Kozakseq1- | RXY (AUG) | 6 |
| where R is a | | |
| purine (A or | | |
| G), Y is either | | |
| C or G, and X | | |
| is any base | | |
| Oct3/4- | MAGHLASDFAFSPPPGGGGDGPGGPEPGWVDPRTWLSFQGPPGGPGI | 7 |
| amino acid | GPGVGPGSEVWGIPPCPPPYEFCGGMAYCGPQVGVGLVPQGGLETSQ | |
| | PEGEAGVGVESNSDGASPEPCTVTPGAVKLEKEKLEQNPEESQDIKA | |
| | LQKELEQFAKLLKQKRITLGYTQADVGLTLGVLFGKVFSQTTICRFE | |
| | ALQLSFKNMCKLRPLLQKWVEEADNNENLQEICKAETLVQARKRKRT | |
| | SIENRVRGNLENLFLQCPKPTLQQISHIAQQLGLEKDVVRVWFCNRR | |
| | QKGKRSSSDYAQREDFEAAGSPFSGGPVSFPLAPGPHFGTPGYGSPH | |
| | FTALYSSVPFPEGEAFPPVSVTTLGSPMHSN | |
| Oct3/4- | AAAAUCCGUUGACCUUAAACGGUCGUGUGGGUUCAAGUCCCUCCACCCCCACGCCGG | 8 |
| nucleic acid | AAACGCAAUAGCCGAAAAACAAAAAACAAAAAAAACAAAAAAAAAACCAAAAAAACA | |
| | AAACACAUUAAAACAGCCUGUGGGUUGAUCCCACCCACAGGCCCAUUGGGCGCUAGC | |
| | ACUCUGGUAUCACGGUACCUUUGUGCGCCUGUUUUAUACCCCCUCCCCCAACUGUAA | |
| | CUUAGAAGUAACACACACCGAUCAACAGUCAGCGUGGCACACCAGCCACGUUUUGAU | |
| | CAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUU | |
| | GCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCA | |
| | UGGGACGCUCUAAUACAGACAUGGUGCGAAGAGUCUAUUGAGCUAGUUGGUAGUCCU | |
| | CCGGCCCCUGAAUGCGGCUAAUCCUAACUGCGGAGCACACACCCUCAAGCCAGAGGG | |
| | CAGUGUGUCGUAACGGGCAACUCUGCAGCGGAACCGACUACUUUGGGUGUCCGUGUU | |
| | UCAUUUUAUUCCUAUACUGGCUGCUUAUGGUGACAAUUGAGAGAUCGUUACCAUAUA | |
| | GCUAUUGGAUUGGCCAUCCGGUGACUAAUAGAGCUAUUAUAUAUCCCUUUGUUGGGU | |
| | UUAUACCACUUAGCUUGAAAGAGGUUAAAACAUUACAAUUCAUUGUUAAGUUGAAUA | |
| | CAGCAAAAUGGCGGGACACCUGGCUUCGGAUUUCGCCUUCUCGCCCCCUCCAGGUGG | |
| | UGGAGGUGAUGGGCCAGGGGGGCCGGAGCCGGGCUGGGUUGAUCCUCGGACCUGGCU | |
| | AAGCUUCCAAGGCCCUCCUGGAGGGCCAGGAAUCGGGCCGGGGGUUGGGCCAGGCUC | |
| | UGAGGUGUGGGGGAUUCCCCCAUGCCCCCCGCCGUAUGAGUUCUGUGGGGGGAUGGC | |
| | GUACUGUGGGCCCCAGGUUGGAGUGGGGCUAGUGCCCCAAGGCGGCUUGGAGACCUC | |
| | UCAGCCUGAGGGCGAAGCAGGAGUCGGGGUGGAGAGCAACUCCGAUGGGGCCUCCCC | |
| | GGAGCCCUGCACCGUCACCCCUGGUGCCGUGAAGCUGGAGAAGGAGAAGCUGGAGCA | |
| | AAACCCGGAGGAGUCCCAGGACAUCAAAGCUCUGCAGAAAGAACUCGAGCAAUUUGC | |
| | CAAGCUCCUGAAGCAGAAGAGGAUCACCCUGGGAUAUACACAGGCCGAUGUGGGGCU | |
| | CACCCUGGGGGUUCUAUUUGGGAAGGUAUUCAGCCAAACGACCAUCUGCCGCUUUGA | |
| | GGCUCUGCAGCUUAGCUUCAAGAACAUGUGUAAGCUGCGGCCCUUGCUGCAGAAGUG | |
| | GGUGGAGGAAGCUGACAACAAUGAAAAUCUUCAGGAGAUAUGCAAAGCAGAAACCCU | |
| | CGUGCAGGCCCGAAAGAGAAAGCGAACCAGUAUCGAGAACCGAGUGAGAGGCAACCU | |
| | GGAGAAUUUGUUCCUGCAGUGCCCGAAACCCACACUGCAGCAGAUCAGCCACAUCGC | |
| | CCAGCAGCUUGGGCUCGAGAAGGAUGUGGUCCGAGUGUGGUUCUGUAACCGGCGCCA | |
| | GAAGGGCAAGCGAUCAAGCAGCGACUAUGCACAACGAGAGGAUUUUGAGGCUGCUGG | |
| | GUCUCCUUUCUCAGGGGGACCAGUGUCCUUUCCUCUGGCCCCAGGGCCCCAUUUUGG | |
| | UACCCCAGGCUAUGGGAGCCCUCACUUCACUGCACUGUACUCCUCGGUCCCUUUCCC | |
| | UGAGGGGGAAGCCUUUCCCCCUGUCUCCGUCACCACUCUGGGCUCUCCCAUGCAUUC | |
| | AAACUGAAAAAAACAAAAAACAAAACGGCUAUUAUGCGUUACCGGCGAGACGCUACG | |
| | GACUU | |
| Klf4-amino | MRQPPGESDMAVSDALLPSFSTFASGPAGREKTLRQAGAPNNRWREE | 9 |
| acid | LSHMKRLPPVLPGRPYDLAAATVATDLESGGAGAACGGSNLAPLPRR | |
| | ETEEFNDLLDLDFILSNSLTHPPESVAATVSSSASASSSSSPSSSGP | |
| | ASAPSTCSFTYPIRAGNDPGVAPGGTGGGLLYGRESAPPPTAPFNLA | |
| | DINDVSPSGGFVAELLRPELDPVYIPPQQPQPPGGGLMGKFVLKASL | |
| | SAPGSEYGSPSVISVSKGSPDGSHPVVVAPYNGGPPRTCPKIKQEAV | |
| | SSCTHLGAGPPLSNGHRPAAHDFPLGRQLPSRTTPTLGLEEVLSSRD | |
| | CHPALPLPPGFHPHPGPNYPSFLPDQMQPQVPPLHYQGQSRGFVARA | |
| | GEPCVCWPHFGTHGMMLIPPSSPLELMPPGSCMPEEPKPKRGRRSWP | |
| | RKRTATHTCDYAGCGKTYTKSSHLKAHLRTHTGEKPYHCDWDGCGWK | |
| | FARSDELTRHYRKHIGHRPFQCQKCDRAFSRSDHLALHMKRHF | |
| Klf4-amino | MRQPPGESDMAVSDALLPSFSTFASGPAGREKTLRQAGAPNNRWREE | 10 |
| acid 2 | LSHMKRLPPVLPGRPYDLAAATVATDLESGGAGAACGGSNLAPLPRR | |
| | ETEEFNDLLDLDFILSNSLTHPPESVAATVSSSASASSSSSPSSSGP | |
| | ASAPSTCSFTYPIRAGNDPGVAPGGTGGGLLYGRESAPPPTAPENLA | |
| | DINDVSPSGGFVAELLRPELDPVYIPPQQPQPPGGGLMGKFVLKASL | |
| | SAPGSEYGSPSVISVSKGSPDGSHPVVVAPYNGGPPRTCPKIKQEAV | |
| | SSCTHLGAGPPLSNGHRPAAHDFPLGRQLPSRTTPTLGLEEVLSSRD | |
| | CHPALPLPPGFHPHPGPNYPSFLPDQMQPQVPPLHYQELMPPGSCMP | |
| | EEPKPKRGRRSWPRKRTATHTCDYAGCGKTYTKSSHLKAHLRTHTGE | |
| | KPYHCDWDGCGWKFARSDELTRHYRKHIGHRPFQCQKCDRAFSRSDH | |
| | LALHMKRHF | |
| Klf4-nucleic | AAAAUCCGUUGACCUUAAACGGUCGUGUGGGUUCAAGUCCCUCCACCCCCACGCCGG | 11 |
| acid | AAACGCAAUAGCCGAAAAACAAAAAACAAAAAAAACAAAAAAAAAACCAAAAAAACA | |
| | AAACACAUUAAAACAGCCUGUGGGUUGAUCCCACCCACAGGCCCAUUGGGCGCUAGC | |
| | ACUCUGGUAUCACGGUACCUUUGUGCGCCUGUUUUAUACCCCCUCCCCCAACUGUAA | |
| | CUUAGAAGUAACACACACCGAUCAACAGUCAGCGUGGCACACCAGCCACGUUUUGAU | |
| | CAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUU | |
| | GCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCA | |
| | UGGGACGCUCUAAUACAGACAUGGUGCGAAGAGUCUAUUGAGCUAGUUGGUAGUCCU | |
| | CCGGCCCCUGAAUGCGGCUAAUCCUAACUGCGGAGCACACACCCUCAAGCCAGAGGG | |
| | CAGUGUGUCGUAACGGGCAACUCUGCAGCGGAACCGACUACUUUGGGUGUCCGUGUU | |
| | UCAUUUUAUUCCUAUACUGGCUGCUUAUGGUGACAAUUGAGAGAUCGUUACCAUAUA | |
| | GCUAUUGGAUUGGCCAUCCGGUGACUAAUAGAGCUAUUAUAUAUCCCUUUGUUGGGU | |
| | UUAUACCACUUAGCUUGAAAGAGGUUAAAACAUUACAAUUCAUUGUUAAGUUGAAUA | |
| | CAGCAAAAUGAGGCAGCCACCUGGCGAGUCUGACAUGGCUGUCAGCGACGCGCUGCU | |
| | CCCAUCUUUCUCCACGUUCGCGUCUGGCCCGGCGGGAAGGGAGAAGACACUGCGUCA | |
| | AGCAGGUGCCCCGAAUAACCGCUGGCGGGAGGAGCUCUCCCACAUGAAGCGACUUCC | |
| | CCCAGUGCUUCCCGGCCGCCCCUAUGACCUGGCGGCGGCGACCGUGGCCACAGACCU | |
| | GGAGAGCGGCGGAGCCGGUGCGGCUUGCGGCGGUAGCAACCUGGCGCCCCUACCUCG | |
| | GAGAGAGACCGAGGAGUUCAACGAUCUCCUGGACCUGGACUUUAUUCUCUCCAAUUC | |
| | GCUGACCCAUCCUCCGGAGUCAGUGGCCGCCACCGUGUCCUCGUCAGCGUCAGCCUC | |
| | CUCUUCGUCGUCGCCGUCGAGCAGCGGCCCUGCCAGCGCGCCCUCCACCUGCAGCUU | |
| | CACCUAUCCGAUCCGGGCCGGGAACGACCCGGGCGUGGCGCCGGGCGGCACGGGCGG | |
| | AGGCCUCCUCUAUGGCAGGGAGUCCGCUCCCCCUCCGACGGCUCCCUUCAACCUGGC | |
| | GGACAUCAACGACGUGAGCCCCUCGGGCGGCUUCGUGGCCGAGCUCCUGCGGCCAGA | |
| | AUUGGACCCGGUGUACAUUCCGCCGCAGCAGCCGCAGCCGCCAGGUGGCGGGCUGAU | |
| | GGGCAAGUUCGUGCUGAAGGCGUCGCUGAGCGCCCCUGGCAGCGAGUACGGCAGCCC | |
| | GUCGGUCAUCAGCGUCAGCAAAGGCAGCCCUGACGGCAGCCACCCGGUGGUGGUGGC | |
| | GCCCUACAACGGCGGGCCGCCGCGCACGUGCCCCAAGAUCAAGCAGGAGGCGGUCUC | |
| | UUCGUGCACCCACUUGGGCGCUGGACCCCCUCUCAGCAAUGGCCACCGGCCGGCUGC | |
| | ACACGACUUCCCCCUGGGGCGGCAGCUCCCCAGCAGGACUACCCCGACCCUGGGUCU | |
| | UGAGGAAGUGCUGAGCAGCAGGGACUGUCACCCUGCCCUGCCGCUUCCUCCCGGCUU | |
| | CCAUCCCCACCCGGGGCCCAAUUACCCAUCCUUCCUGCCCGAUCAGAUGCAGCCGCA | |
| | AGUCCCGCCGCUCCAUUACCAAGAGCUCAUGCCACCCGGUUCCUGCAUGCCAGAGGA | |
| | GCCCAAGCCAAAGAGGGGAAGACGAUCGUGGCCCCGGAAAAGGACCGCCACCCACAC | |
| | UUGUGAUUACGCGGGCUGCGGCAAAACCUACACAAAGAGUUCCCAUCUCAAGGCACA | |
| | CCUGCGAACCCACACAGGUGAGAAACCUUACCACUGUGACUGGGACGGCUGUGGAUG | |
| | GAAAUUCGCCCGCUCAGAUGAACUGACCAGGCACUACCGUAAACACACGGGGCACCG | |
| | CCCGUUCCAGUGCCAAAAAUGCGACCGAGCAUUUUCCAGGUCGGACCACCUCGCCUU | |
| | ACACAUGAAGAGGCAUUUUUAAAAAAAACAAAAAACAAAACGGCUAUUAUGCGUUAC | |
| | CGGCGAGACGCUACGGACUU | |
| Sox2-amino | MYNMMETELKPPGPQQTSGGGGGNSTAAAAGGNQKNSPDRVKRPMNA | 12 |
| acid | FMVWSRGQRRKMAQENPKMHNSEISKRLGAEWKLLSETEKRPFIDEA | |
| | KRLRALHMKEHPDYKYRPRRKTKTLMKKDKYTLPGGLLAPGGNSMAS | |
| | GVGVGAGLGAGVNORMDSYAHMNGWSNGSYSMMQDQLGYPQHPGLNA | |
| | HGAAQMQPMHRYDVSALQYNSMTSSQTYMNGSPTYSMSYSQQGTPGM | |
| | ALGSMGSVVKSEASSSPPVVTSSSHSRAPCQAGDLRDMISMYLPGAE | |
| | VPEPAAPSRLHMSQHYQSGPVPGTAINGTLPLSHM | |
| Sox2- | AAAAUCCGUUGACCUUAAACGGUCGUGUGGGUUCAAGUCCCUCCACCCCCACGCCGG | 13 |
| nucleic acid | AAACGCAAUAGCCGAAAAACAAAAAACAAAAAAAACAAAAAAAAAACCAAAAAAACA | |
| | AAACACAUUAAAACAGCCUGUGGGUUGAUCCCACCCACAGGCCCAUUGGGCGCUAGC | |
| | ACUCUGGUAUCACGGUACCUUUGUGCGCCUGUUUUAUACCCCCUCCCCCAACUGUAA | |
| | CUUAGAAGUAACACACACCGAUCAACAGUCAGCGUGGCACACCAGCCACGUUUUGAU | |
| | CAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUU | |
| | GCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCA | |
| | UGGGACGCUCUAAUACAGACAUGGUGCGAAGAGUCUAUUGAGCUAGUUGGUAGUCCU | |
| | CCGGCCCCUGAAUGCGGCUAAUCCUAACUGCGGAGCACACACCCUCAAGCCAGAGGG | |
| | CAGUGUGUCGUAACGGGCAACUCUGCAGCGGAACCGACUACUUUGGGUGUCCGUGUU | |
| | UCAUUUUAUUCCUAUACUGGCUGCUUAUGGUGACAAUUGAGAGAUCGUUACCAUAUA | |
| | GCUAUUGGAUUGGCCAUCCGGUGACUAAUAGAGCUAUUAUAUAUCCCUUUGUUGGGU | |
| | UUAUACCACUUAGCUUGAAAGAGGUUAAAACAUUACAAUUCAUUGUUAAGUUGAAUA | |
| | CAGCAAAAUGUACAACAUGAUGGAGACGGAGCUGAAGCCGCCGGGCCCGCAGCAAAC | |
| | UUCGGGGGGCGGCGGCGGCAACUCCACCGCGGCGGCGGCCGGCGGCAACCAGAAAAA | |
| | CAGCCCGGACCGCGUCAAGCGGCCCAUGAAUGCCUUCAUGGUGUGGUCCCGCGGGCA | |
| | GCGGCGCAAGAUGGCCCAGGAGAACCCCAAGAUGCACAACUCGGAGAUCAGCAAGCG | |
| | CCUGGGCGCCGAGUGGAAACUUUUGUCGGAGACGGAGAAGCGGCCGUUCAUCGACGA | |
| | GGCUAAGCGGCUGCGAGCGCUGCACAUGAAGGAGCACCCGGAUUAUAAAUACCGGCC | |
| | CCGGCGGAAAACCAAGACGCUCAUGAAGAAGGAUAAGUACACGCUGCCCGGCGGGCU | |
| | GCUGGCCCCCGGCGGCAAUAGCAUGGCGAGCGGGGUCGGGGUGGGCGCCGGCCUGGG | |
| | CGCGGGCGUGAACCAGCGCAUGGACAGUUACGCGCACAUGAACGGCUGGAGCAACGG | |
| | CAGCUACAGCAUGAUGCAGGACCAGCUGGGCUACCCGCAGCACCCGGGCCUCAAUGC | |
| | GCACGGCGCAGCGCAGAUGCAGCCCAUGCACCGCUACGACGUGAGCGCCCUGCAGUA | |
| | CAACUCCAUGACCAGCUCGCAGACCUACAUGAACGGCUCGCCCACCUACAGCAUGUC | |
| | CUACUCGCAGCAGGGCACCCCUGGCAUGGCUCUUGGCUCCAUGGGUUCGGUGGUCAA | |
| | GUCCGAGGCCAGCUCCAGCCCCCCUGUGGUUACCUCUUCCUCCCACUCCAGGGCGCC | |
| | CUGCCAGGCCGGGGACCUCCGGGACAUGAUCAGCAUGUAUCUCCCCGGCGCCGAGGU | |
| | GCCGGAACCCGCCGCCCCCAGCAGACUUCACAUGUCCCAGCACUACCAGAGCGGCCC | |
| | GGUGCCCGGCACGGCCAUUAACGGCACACUGCCCCUCUCACACAUGUGAAAAAAACA | |
| | AAAAACAAAACGGCUAUUAUGCGUUACCGGCGAGACGCUACGGACUU | |
| NanogAA- | MSVDPACPQSLPCFEASDCKESSPMPVICGPEENYPSLOMSSAEMPH | 14 |
| amino acid 1 | TETVSPLPSSMDLLIQDSPDSSTSPKGKQPTSAEKSVAKKEDKVPVK | |
| | KOKTRTVFSSTQLCVLNDRFOROKYLSLOOMQELSNILNLSYKQVKT | |
| | WFQNQRMKSKRWQKNNWPKNSNGVTQGCLVNPTGNLPMWSNQTWNNS | |
| | TWSNQTQNIQSWSNHSWNTQTWCTQSWNNQAWNSPFYNCGEESLQSC | |
| | MQFQPNSPASDLEAALEAAGEGLNVIQQTTRYFSTPQTMDLFLNYSM | |
| | NMQPEDV | |
| Nanog- | MSVDPACPQSLPCFEASDCKESSPMPVICGPEENYPSLOMSSAEMPH | 15 |
| amino acid 2 | TETVSPLPSSMDLLIQDSPDSSTSPKGKOPTSAEKSVAKKEDKVPVK | |
| | KOKTRTVFSSTQLCVLNDRFORQKYLSLOOMQELSNILNLSYKQVKT | |
| | WFQNQRMKSKRWQKNNWPKNSNGVTOKASAPTYPSLYSSYHQGCLVN | |
| | PTGNLPMWSNQTWNNSTWSNQTONIQSWSNHSWNTQTWCTOSWNNQA | |
| | WNSPFYNCGEESLOSCMQFQPNSPASDLEAALEAAGEGLNVIQQTTR | |
| | YFSTPQTMDLFLNYSMNMOPEDV | |
| Nanog- | AAAAUCCGUUGACCUUAAACGGUCGUGUGGGUUCAAGUCCCUCCACCCCCACGCCGG | 16 |
| nucleic acid | AAACGCAAUAGCCGAAAAACAAAAAACAAAAAAAACAAAAAAAAAACCAAAAAAACA | |
| | AAACACAUUAAAACAGCCUGUGGGUUGAUCCCACCCACAGGCCCAUUGGGCGCUAGC | |
| | ACUCUGGUAUCACGGUACCUUUGUGCGCCUGUUUUAUACCCCCUCCCCCAACUGUAA | |
| | CUUAGAAGUAACACACACCGAUCAACAGUCAGCGUGGCACACCAGCCACGUUUUGAU | |
| | CAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUU | |
| | GCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCA | |
| | UGGGACGCUCUAAUACAGACAUGGUGCGAAGAGUCUAUUGAGCUAGUUGGUAGUCCU | |
| | CCGGCCCCUGAAUGCGGCUAAUCCUAACUGCGGAGCACACACCCUCAAGCCAGAGGG | |
| | CAGUGUGUCGUAACGGGCAACUCUGCAGCGGAACCGACUACUUUGGGUGUCCGUGUU | |
| | UCAUUUUAUUCCUAUACUGGCUGCUUAUGGUGACAAUUGAGAGAUCGUUACCAUAUA | |
| | GCUAUUGGAUUGGCCAUCCGGUGACUAAUAGAGCUAUUAUAUAUCCCUUUGUUGGGU | |
| | UUAUACCACUUAGCUUGAAAGAGGUUAAAACAUUACAAUUCAUUGUUAAGUUGAAUA | |
| | CAGCAAAAUGAGUGUGGAUCCAGCUUGUCCCCAAAGCUUGCCUUGCUUUGAAGCAUC | |
| | CGACUGUAAAGAAUCUUCACCUAUGCCUGUGAUUUGUGGGCCUGAAGAAAACUAUCC | |
| | AUCCUUGCAAAUGUCUUCUGCUGAGAUGCCUCACACGGAGACUGUCUCUCCUCUUCC | |
| | UUCCUCCAUGGAUCUGCUUAUUCAGGACAGCCCUGAUUCUUCCACCAGUCCCAAAGG | |
| | CAAACAACCCACUUCUGCAGAGAAGAGUGUCGCAAAAAAGGAAGACAAGGUCCCGGU | |
| | CAAGAAACAGAAGACCAGAACUGUGUUCUCUUCCACCCAGCUGUGUGUACUCAAUGA | |
| | UAGAUUUCAGAGACAGAAAUACCUCAGCCUCCAGCAGAUGCAAGAACUCUCCAACAU | |
| | CCUGAACCUCAGCUACAAACAGGUGAAGACCUGGUUCCAGAACCAGAGAAUGAAAUC | |
| | UAAGAGGUGGCAGAAAAACAACUGGCCGAAGAAUAGCAAUGGUGUGACGCAGAAGGC | |
| | CUCAGCACCUACCUACCCCAGCCUUUACUCUUCCUACCACCAGGGAUGCCUGGUGAA | |
| | CCCGACUGGGAACCUUCCAAUGUGGAGCAACCAGACCUGGAACAAUUCAACCUGGAG | |
| | CAACCAGACCCAGAACAUCCAGUCCUGGAGCAACCACUCCUGGAACACUCAGACCUG | |
| | GUGCACCCAAUCCUGGAACAAUCAGGCCUGGAACAGUCCCUUCUAUAACUGUGGAGA | |
| | GGAAUCUCUGCAGUCCUGCAUGCAGUUCCAGCCAAAUUCUCCUGCCAGUGACUUGGA | |
| | GGCUGCCUUGGAAGCUGCUGGGGAAGGCCUUAAUGUAAUACAGCAGACCACUAGGUA | |
| | UUUUAGUACUCCACAAACCAUGGAUUUAUUCCUAAACUACUCCAUGAACAUGCAACC | |
| | UGAAGACGUGUGAAAAAAACAAAAAACAAAACGGCUAUUAUGCGUUACCGGCGAGAC | |
| | GCUACGGACUU | |
| Lin28A- | MGSVSNQQFAGGCAKAAEEAPEEAPEDAARAADEPOLLHGAGICKWF | 17 |
| amino acid | NVRMGFGFLSMTARAGVALDPPVDVFVHQSKLHMEGFRSLKEGEAVE | |
| | FTFKKSAKGLESIRVTGPGGVFCIGSERRPKGKSMQKRRSKGDRCYN | |
| | CGGLDHHAKECKLPPQPKKCHFCOSISHMVASCPLKAQQGPSAQGKP | |
| | TYFREEEEEIHSPILLPEAQN | |
| Lin28A- | AAAAUCCGUUGACCUUAAACGGUCGUGUGGGUUCAAGUCCCUCCACCCCCACGCCGG | 18 |
| nucleic acid | AAACGCAAUAGCCGAAAAACAAAAAACAAAAAAAACAAAAAAAAAACCAAAAAAACA | |
| | AAACACAUUAAAACAGCCUGUGGGUUGAUCCCACCCACAGGCCCAUUGGGCGCUAGC | |
| | ACUCUGGUAUCACGGUACCUUUGUGCGCCUGUUUUAUACCCCCUCCCCCAACUGUAA | |
| | CUUAGAAGUAACACACACCGAUCAACAGUCAGCGUGGCACACCAGCCACGUUUUGAU | |
| | CAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUU | |
| | GCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCA | |
| | UGGGACGCUCUAAUACAGACAUGGUGCGAAGAGUCUAUUGAGCUAGUUGGUAGUCCU | |
| | CCGGCCCCUGAAUGCGGCUAAUCCUAACUGCGGAGCACACACCCUCAAGCCAGAGGG | |
| | CAGUGUGUCGUAACGGGCAACUCUGCAGCGGAACCGACUACUUUGGGUGUCCGUGUU | |
| | UCAUUUUAUUCCUAUACUGGCUGCUUAUGGUGACAAUUGAGAGAUCGUUACCAUAUA | |
| | GCUAUUGGAUUGGCCAUCCGGUGACUAAUAGAGCUAUUAUAUAUCCCUUUGUUGGGU | |
| | UUAUACCACUUAGCUUGAAAGAGGUUAAAACAUUACAAUUCAUUGUUAAGUUGAAUA | |
| | CAGCAAAAUGGGCUCCGUGUCCAACCAGCAGUUUGCAGGUGGCUGCGCCAAGGCGGC | |
| | AGAAGAGGCGCCCGAGGAGGCGCCGGAGGACGCGGCCCGGGCGGCGGACGAGCCUCA | |
| | GCUGCUGCACGGUGCGGGCAUCUGUAAGUGGUUCAACGUGCGCAUGGGGUUCGGCUU | |
| | CCUGUCCAUGACCGCCCGCGCCGGGGUCGCGCUCGACCCCCCAGUGGAUGUCUUUGU | |
| | GCACCAGAGUAAGCUGCACAUGGAAGGGUUCCGGAGCUUGAAGGAGGGUGAGGCAGU | |
| | GGAGUUCACCUUUAAGAAGUCAGCCAAGGGUCUGGAAUCCAUCCGUGUCACCGGACC | |
| | UGGUGGAGUAUUCUGUAUUGGGAGUGAGAGGCGGCCAAAAGGAAAGAGCAUGCAGAA | |
| | GCGCAGAUCAAAAGGAGACAGGUGCUACAACUGUGGAGGUCUAGAUCAUCAUGCCAA | |
| | GGAAUGCAAGCUGCCACCCCAGCCCAAGAAGUGCCACUUCUGCCAGAGCAUCAGCCA | |
| | UAUGGUAGCCUCAUGUCCGCUGAAGGCCCAGCAGGGCCCUAGUGCACAGGGAAAGCC | |
| | AACCUACUUUCGAGAGGAAGAAGAAGAAAUCCACAGCCCUACCCUGCUCCCGGAGGC | |
| | ACAGAAUUGAAAAAAACAAAAAACAAAACGGCUAUUAUGCGUUACCGGCGAGACGCU | |
| | ACGGACUU | |
| c-Myc- | MDFFRVVENQQPPATMPLNVSFTNRNYDLDYDSVQPYFYCDEEENFY | 19 |
| amino acid 1 | QQQQQSELOPPAPSEDIWKKFELLPTPPLSPSRRSGLCSPSYVAVTP | |
| | FSLRGDNDGGGGSFSTADQLEMVTELLGGDMVNOSFICDPDDETFIK | |
| | NIIIQDCMWSGFSAAAKLVSEKLASYQAARKDSGSPNPARGHSVCST | |
| | SSLYLQDLSAAASECIDPSVVFPYPLNDSSSPKSCASODSSAFSPSS | |
| | DSLLSSTESSPQGSPEPLVLHEETPPTTSSDSEEEQEDEEEIDVVSV | |
| | EKRQAPGKRSESGSPSAGGHSKPPHSPLVLKRCHVSTHOHNYAAPPS | |
| | TRKDYPAAKRVKLDSVRVLRQISNNRKCTSPRSSDTEENVKRRTHNV | |
| | LERQRRNELKRSFFALRDQIPELENNEKAPKVVILKKATAYILSVQA | |
| | EEQKLISEEDLLRKRREQLKHKLEQLRNSCA | |
| c-Myc- | MDFFRVVENQPPATMPLNVSFTNRNYDLDYDSVQPYFYCDEEENFYQ | 20 |
| amino acid 2 | QQQQSELQPPAPSEDIWKKFELLPTPPLSPSRRSGLCSPSYVAVTPF | |
| | SLRGDNDGGGGSFSTADQLEMVTELLGGDMVNQSFICDPDDETFIKN | |
| | IIIQDCMWSGFSAAAKLVSEKLASYQAARKDSGSPNPARGHSVCSTS | |
| | SLYLQDLSAAASECIDPSVVFPYPLNDSSSPKSCASQDSSAFSPSSD | |
| | SLLSSTESSPQGSPEPLVLHEETPPTTSSDSEEEQEDEEEIDVVSVE | |
| | KRQAPGKRSESGSPSAGGHSKPPHSPLVLKRCHVSTHOHNYAAPPST | |
| | RKDYPAAKRVKLDSVRVLRQISNNRKCTSPRSSDTEENVKRRTHNVL | |
| | ERQRRNELKRSFFALRDQIPELENNEKAPKVVILKKATAYILSVQAE | |
| | EQKLISEEDLLRKRREQLKHKLEQLRNSCA | |
| c-Myc- | AAAAUCCGUUGACCUUAAACGGUCGUGUGGGUUCAAGUCCCUCCACCCCCACGCCGG | 21 |
| nucleic acid | AAACGCAAUAGCCGAAAAACAAAAAACAAAAAAAACAAAAAAAAAACCAAAAAAACA | |
| | AAACACAUUAAAACAGCCUGUGGGUUGAUCCCACCCACAGGCCCAUUGGGCGCUAGC | |
| | ACUCUGGUAUCACGGUACCUUUGUGCGCCUGUUUUAUACCCCCUCCCCCAACUGUAA | |
| | CUUAGAAGUAACACACACCGAUCAACAGUCAGCGUGGCACACCAGCCACGUUUUGAU | |
| | CAAGCACUUCUGUUACCCCGGACUGAGUAUCAAUAGACUGCUCACGCGGUUGAAGGA | |
| | GAAAGCGUUCGUUAUCCGGCCAACUACUUCGAAAAACCUAGUAACACCGUGGAAGUU | |
| | GCAGAGUGUUUCGCUCAGCACUACCCCAGUGUAGAUCAGGUCGAUGAGUCACCGCAU | |
| | UCCCCACGGGCGACCGUGGCGGUGGCUGCGUUGGCGGCCUGCCCAUGGGGAAACCCA | |
| | UGGGACGCUCUAAUACAGACAUGGUGCGAAGAGUCUAUUGAGCUAGUUGGUAGUCCU | |
| | CCGGCCCCUGAAUGCGGCUAAUCCUAACUGCGGAGCACACACCCUCAAGCCAGAGGG | |
| | CAGUGUGUCGUAACGGGCAACUCUGCAGCGGAACCGACUACUUUGGGUGUCCGUGUU | |
| | UCAUUUUAUUCCUAUACUGGCUGCUUAUGGUGACAAUUGAGAGAUCGUUACCAUAUA | |
| | GCUAUUGGAUUGGCCAUCCGGUGACUAAUAGAGCUAUUAUAUAUCCCUUUGUUGGGU | |
| | UUAUACCACUUAGCUUGAAAGAGGUUAAAACAUUACAAUUCAUUGUUAAGUUGAAUA | |
| | CAGCAAAAUGCCCCUCAACGUUAGCUUCACCAACAGGAACUAUGACCUCGACUACGA | |
| | CUCGGUGCAGCCGUAUUUCUACUGCGACGAGGAGGAGAACUUCUACCAGCAGCAGCA | |
| | GCAGAGCGAGCUGCAGCCCCCGGCGCCCAGCGAGGAUAUCUGGAAGAAAUUCGAGCU | |
| | GCUGCCCACCCCGCCCCUGUCCCCUAGCCGCCGCUCCGGGCUCUGCUCGCCCUCCUA | |
| | CGUUGCGGUCACACCCUUCUCCCUUCGGGGAGACAACGACGGCGGUGGCGGGAGCUU | |
| | CUCCACGGCCGACCAGCUGGAGAUGGUGACCGAGCUGCUGGGAGGAGACAUGGUGAA | |
| | CCAGAGUUUCAUCUGCGACCCGGACGACGAGACCUUCAUCAAAAACAUCAUCAUCCA | |
| | GGACUGUAUGUGGAGCGGCUUCUCGGCCGCCGCCAAGCUCGUCUCAGAGAAGCUGGC | |
| | CUCCUACCAGGCUGCGCGCAAAGACAGCGGCAGCCCGAACCCCGCCCGCGGCCACAG | |
| | CGUCUGCUCCACCUCCAGCUUGUACCUGCAGGAUCUGAGCGCCGCCGCCUCAGAGUG | |
| | CAUCGACCCCUCGGUGGUCUUCCCCUACCCUCUCAACGACAGCAGCUCGCCCAAGUC | |
| | CUGCGCCUCGCAAGACUCCAGCGCCUUCUCUCCGUCCUCGGAUUCUCUGCUCUCCUC | |
| | GACGGAGUCCUCCCCGCAGGGCAGCCCCGAGCCCCUGGUGCUCCAUGAGGAGACACC | |
| | GCCCACCACCAGCAGCGACUCUGAGGAGGAACAAGAAGAUGAGGAAGAAAUCGAUGU | |
| | UGUUUCUGUGGAAAAGAGGCAGGCUCCUGGCAAAAGGUCAGAGUCUGGAUCACCUUC | |
| | UGCUGGAGGCCACAGCAAACCUCCUCACAGCCCACUGGUCCUCAAGAGGUGCCACGU | |
| | CUCCACACAUCAGCACAACUACGCAGCGCCUCCCUCCACUCGGAAGGACUAUCCUGC | |
| | UGCCAAGAGGGUCAAGUUGGACAGUGUCAGAGUCCUGAGACAGAUCAGCAACAACCG | |
| | AAAAUGCACCAGCCCCAGGUCCUCGGACACCGAGGAGAAUGUCAAGAGGCGAACACA | |
| | CAACGUCUUGGAGCGCCAGAGGAGGAACGAGCUAAAACGGAGCUUUUUUGCCCUGCG | |
| | UGACCAGAUCCCGGAGUUGGAAAACAAUGAAAAGGCCCCCAAGGUAGUUAUCCUUAA | |
| | AAAAGCCACAGCAUACAUCCUGUCCGUCCAAGCAGAGGAGCAAAAGCUCAUUUCUGA | |
| | AGAGGACUUGUUGCGGAAACGACGAGAACAGUUGAAACACAAACUUGAACAGCUACG | |
| | GAACUCUUGUGCGUAAAAAAAACAAAAAACAAAACGGCUAUUAUGCGUUACCGGCGA | |
| | GACGCUACGGACUU | |
| L-Myc- | MCVCAGCRAAPSRRGAGPLQVAGGWSEGADMDYDSYQHYFYDYDCGE | 22 |
| amino acid 1 | DFYRSTAPSEDIWKKFELVPSPPTSPPWGLGPGAGDPAPGIGPPEPW | |
| | PGGCTGDEAESRGHSKGWGRNYASIIRRDCMWSGFSARERLERAVSD | |
| | RLAPGAPRGNPPKASAAPDCTPSLEAGNPAPAAPCPLGEPKTQACSG | |
| | SESPSDSGKDLPEPSKRGPPHGWPKLCPCLRSGIGSSQALGPSPPLF | |
| | G | |
| L-Myc- | MDYDSYQHYFYDYDCGEDFYRSTAPSEDIWKKFELVPSPPTSPPWGL | 23 |
| amino acid 2 | GPGAGDPAPGIGPPEPWPGGCTGDEAESRGHSKGWGRNYASIIRRDC | |
| | MWSGFSARERLERAVSDRLAPGAPRGNPPKASAAPDCTPSLEAGNPA | |
| | PAAPCPLGEPKTQACSGSESPSDSENEEIDVVTVEKRQSLGIRKPVT | |
| | ITVRADPLDPCMKHFHISIHQQQHNYAARFPPESCSQEEASERGPQE | |
| | EVLERDAAGEKEDEEDEEIVSPPPVESEAAQSCHPKPVSSDTEDVTK | |
| | RKNHNFLERKRRNDLRSRFLALRDQVPTLASCSKAPKVVILSKALEY | |
| | LOALVGAEKRMATEKROLRCRQQQLQKRIAYLIGY | |
| L-Myc- | MCVCAGCRAAPSRRGAGPLOVAGGWSEGADMDYDSYQHYFYDYDCGE | 24 |
| amino acid 3 | DFYRSTAPSEDIWKKFELVPSPPTSPPWGLGPGAGDPAPGIGPPEPW | |
| | PGGCTGDEAESRGHSKGWGRNYASIIRRDCMWSGFSARERLERAVSD | |
| | RLAPGAPRGNPPKASAAPDCTPSLEAGNPAPAAPCPLGEPKTQACSG | |
| | SESPSDSENEEIDVVTVEKRQSLGIRKPVTITVRADPLDPCMKHFHI | |
| | SIHQQQHNYAARFPPESCSQEEASERGPQEEVLERDAAGEKEDEEDE | |
| | EIVSPPPVESEAAQSCHPKPVSSDTEDVTKRKNHNFLERKRRNDLRS | |
| | RFLALRDQVPTLASCSKAPKVVILSKALEYLQALVGAEKRMATEKRQ | |
| | LRCROQQLQKRIAYLTGY | |
| L-Myc- | AUGUGCGUGUGUGCUGGCUGCCGGGCUGCCCCGAGCCGGCGGGGAGC | 25 |
| nucleic acid | CGGUCCGCUCCAGGUGGCGGGCGGCUGGAGCGAGGGAGCGGACAUGG | |
| | ACUACGACUCGUACCAGCACUAUUUCUACGACUAUGACUGCGGGGAG | |
| | GAUUUCUACCGCUCCACGGCGCCCAGCGAGGACAUCUGGAAGAAAUU | |
| | CGAGCUGGUGCCAUCGCCCCCCACGUCGCCGCCCUGGGGCUUGGGUC | |
| | CCGGCGCAGGGGACCCGGCCCCCGGGAUUGGUCCCCCGGAGCCGUGG | |
| | CCCGGAGGGUGCACCGGAGACGAAGCGGAAUCCCGGGGCCACUCGAA | |
| | AGGCUGGGGCAGGAACUACGCCUCCAUCAUACGCCGUGACUGCAUGU | |
| | GGAGCGGCUUCUCGGCCCGGGAACGGCUGGAGAGAGCUGUGAGCGAC | |
| | CGGCUCGCUCCUGGCGCGCCCCGGGGGAACCCGCCCAAGGCGUCCGC | |
| | CGCCCCGGACUGCACUCCCAGCCUCGAAGCCGGCAACCCGGCGCCCG | |
| | CCGCCCCCUGUCCGCUGGGCGAACCCAAGACCCAGGCCUGCUCCGGG | |
| | UCCGAGAGCCCAAGCGACUCGGAGAAUGAAGAAAUUGAUGUUGUGAC | |
| | AGUAGAGAAGAGGCAGUCUCUGGGUAUUCGGAAGCCGGUCACCAUCA | |
| | CGGUGCGAGCAGACCCCCUGGAUCCCUGCAUGAAGCAUUUCCACAUC | |
| | UCCAUCCAUCAGCAACAGCACAACUAUGCUGCCCGUUUUCCUCCAGA | |
| | AAGCUGCUCCCAAGAAGAGGCUUCAGAGAGGGGUCCCCAAGAAGAGG | |
| | UUCUGGAGAGAGAUGCUGCAGGGGAAAAGGAAGAUGAGGAGGAUGAA | |
| | GAGAUUGUGAGUCCCCCACCUGUAGAAAGUGAGGCUGCCCAGUCCUG | |
| | CCACCCCAAACCUGUCAGUUCUGAUACUGAGGAUGUGACCAAGAGGA | |
| | AGAAUCACAACUUCCUGGAGCGCAAGAGGCGGAAUGACCUGCGUUCG | |
| | CGAUUCUUGGCGCUGAGGGACCAGGUGCCCACCCUGGCCAGCUGCUC | |
| | CAAGGCCCCCAAAGUAGUGAUCCUAAGCAAGGCCUUGGAAUACUUGC | |
| | AAGCCCUGGUGGGGGCUGAGAAGAGGAUGGCUACAGAGAAAAGACAG | |
| | CUCCGAUGCCGGCAGCAGCAGUUGCAGAAAAGAAUUGCAUACCUCAC | |
| | UGGCUACUAA | |
| B18R from | MTMKMMVHIYFVSLLLLLFHSYAIDIENEITEFFNKMRDTLPAKDSK | 26 |
| vaccinia virus | WLNPACMFGGTMNDIAALGEPFSAKCPPIEDSLLSHRYKDYVVKWER | |
| | LEKNRRRQVSNKRVKHGDLWIANYTSKFSNRRYLCTVTTKNGDCVQG | |
| | IVRSHIRKPPSCIPKTYELGTHDKYGIDLYCGILYAKHYNNITWYKD | |
| | NKEINIDDIKYSQTGKELIIHNPELEDSGRYDCYVHYDDVRIKNDIV | |
| | VSRCKILTVIPSQDHRFKLILDPKINVTIGEPANITCTAVSTSLLID | |
| | DVLIEWENPSGWLIGFDFDVYSVLTSRGGITEATLYFENVTEEYIGN | |
| | TYKCRGHNYYFEKTLTTTVVLE | |
| E3 from | MSKIYIDERSNAEIVCEAIKTIGIEGATAAQLTROLNMEKREVNKAL | 27 |
| vaccina virus | YDLORSAMVYSSDDIPPRWFMTTEADEADADAMSDVIIDDVSREKSM | |
| | REDHKSFDDVIPAKKIIDWKGANPVTVINEYCQITRRDWSFRIESVG | |
| | PSNSPTFYACVDIDGRVFDKADGKSKRDAKNNAAKLAVDKLLGYVII | |
| | RF | |
| K3 from | MLAFCYSLPNAGDVIKGRVYEKDYALYIYLFDYPHFEAILAESVKMH | 28 |
| vaccinia virus | MDRYVEYRDKLVGKTVKVKVIRVDYTKGYIDVNYKRMCRHQ | |
| circBIRC6 | CUAAACCAGGUGGACAGGUGAAAUGUCAGUAUAUCUCUGCUGUGGAUAAAGUUAUAU | 29 |
| | UUGUGGAUGAUUAUGCAGUAGGGUGUAGGAAGGACCUUAAUGGAAUCUUGUUGUUAG | |
| | ACACUGCUCUGCAAACUCCAGUUUCAAAGCAGGAUGAUGUGGUUCAGCUUGAAUUAC | |
| | CCGUUACAGAGGCACAGCAGCUCUUAUCAGCAUGUUUAGAAAAGGUAGAUAUUUCUA | |
| | GUACAGAGGGUUAUGAUUUGUUCAUCACACAGCUCAAAGAUGGUUUAAAAAAUACAU | |
| | CUCAUGAGACUGCAGCAAACCACAAAGUUGCUAAG | |
| circCORO1C | AAAAAUAUGCAGGAACCAAUUGCUCUUCAUGAGAUGGACACUAGCAAUGGGGUGUUG | 30 |
| | CUGCCUUUCUAUGACCCUGACACCAGCAUCAUUUACUUAUGUGGAAAGGGUGACAGC | |
| | AGUAUUCGCUAUUUUGAGAUCACGGAUGAAUCCCCGUACGUCCACUACCUCAACACA | |
| | UUCAGCAGCAAGGAGCCUCAGAGAGGGAUGGGUUACAUGCCCAAGAGGGGACUUGAU | |
| | GUUAACAAAUGUGAGAUUGCCAG | |
| circMAN1A2 | GGAAGAGGAAGAACGUCUGAGAAAUAAAAUUCGAGCUGAUCAUGAGAAGGCCUUGGA | 31 |
| | AGAAGCAAAAGAAAAAUUAAGAAAGUCAAGAGAGGAAAUUCGAGCAGAAAUUCAGAC | |
| | AGAGAAAAAUAAGGUAGUCCAAGAAAUGAAGAUAAAAGAGAACAAGCCACUGCCACC | |
| | AGUCCCUAUUCCCAACCUUGUAGGAAUACGUGGUGGAGACCCAGAAGAUAAUGACAU | |
| | AAGAGAGAAAAGGGAAAAAAUUAAAGAGAUGAUGAAACAUGCUUGGGAUAACUAUAG | |
| | GACAUAUGGGUGGGGACAUAAUGAACUCAGACCUAUUGCAAGGAAAGGACACUCCCC | |
| | UAACAUAUUUG | |

### Further Numbered Embodiments

Further numbered embodiments of the disclosure are as follows:

Embodiment 1. A method of producing an induced pluripotent stem cell (iPSC), the method comprising contacting a blood cell in suspension with a circular RNA encoding at least one reprogramming factor selected from Oct3/4, Klf4, Sox2, Nanog, Lin28, c-Myc, or L-Myc, or a fragment or variant thereof, and maintaining the cell under conditions under which the iPSC is obtained.

Embodiment 2. The method of Embodiment 1, wherein the blood cell is selected from a T cell, a B cell, a natural killer (NK) cell, a natural killer T (NKT) cell, a peripheral blood mononuclear cell (PBMC), and a cord blood mononuclear cell (CBMC).

Embodiment 3. The method of Embodiment 1, wherein the blood cell is selected from a T cell and an NK cell.

Embodiment 4. A method of producing an induced pluripotent stem cell (iPSC), the method comprising contacting a CD34+ cell in suspension with a circular RNA encoding at least one reprogramming factor selected from Oct3/4, Klf4, Sox2, Nanog, Lin28, c-Myc, or L-Myc, or a fragment or variant thereof, and maintaining the cell under conditions under which the iPSC is obtained.

Embodiment 5. The method of Embodiment any one of Embodiments 1-4, wherein the at least one reprogramming factor is a human or a humanized reprogramming factor.

Embodiment 6. The method of Embodiment any one of Embodiments 1-5, wherein the at least one reprogramming factor is Oct3/4, and wherein the Oct3/4 has the amino acid sequence of SEQ ID NO: 7, or a sequence at least 90% or at least 95% identical thereto.

Embodiment 7. The method of Embodiment 3, wherein the circular RNA comprises a nucleic acid sequence of SEQ ID NO: 8, or a sequence at least 90% or at least 95% identical thereto.

Embodiment 8. The method of any one of Embodiments 1-5, wherein the at least one reprogramming factor is Klf4, and wherein the Klf4 has the amino acid sequence of SEQ ID NO: 9 or 10, or a sequence at least 90% or at least 95% identical thereto.

Embodiment 9. The method of Embodiment 8, wherein the circular RNA comprises a nucleic acid sequence of SEQ ID NO: 11, or a sequence at least 90% or at least 95% identical thereto.

Embodiment 10. The method of any one of Embodiments 1-5, wherein the at least one reprogramming factor is Sox2, and wherein Sox2 has the amino acid sequence of SEQ ID NO: 12, or a sequence at least 90% or at least 95% identical thereto.

Embodiment 11. The method of Embodiment 10, wherein the circular RNA comprises a nucleic acid sequence of SEQ ID NO: 13, or a sequence at least 90% or at least 95% identical thereto.

Embodiment 12. The method of any one of Embodiments 1-5, wherein the at least one reprogramming factor is Nanog, and wherein the Nanog has the amino acid sequence of SEQ ID NO: 14 or 15, or a sequence at least 90% or at least 95% identical thereto.

Embodiment 13. The method of Embodiment 12, wherein the circular RNA comprises a nucleic acid sequence of SEQ ID NO: 16, or a sequence at least 90% or at least 95% identical thereto.

Embodiment 14. The method of any one of Embodiments 1-5, wherein the at least one reprogramming factor is Lin28A, and wherein the Lin28A has the amino acid sequence of SEQ ID NO: 17, or a sequence at least 90% or at least 95% identical thereto.

Embodiment 15. The method of Embodiment 14, wherein the circular RNA comprises a nucleic acid sequence of SEQ ID NO: 18, or a sequence at least 90% or at least 95% identical thereto.

Embodiment 16. The method of any one of Embodiments 1-5, wherein the at least one reprogramming factor is c-Myc, and wherein the c-Myc has the amino acid sequence of SEQ ID NO: 19 or 20, or a sequence at least 90% or at least 95% identical thereto.

Embodiment 17. The method of Embodiment 16, wherein the circular RNA comprises a nucleic acid sequence of SEQ ID NO: 21, or a sequence at least 90% or at least 95% identical thereto.

Embodiment 18. The method of any one of Embodiments 1-5, wherein the at least one reprogramming factor is L-Myc, and wherein the L-Myc has the amino acid sequence of any one of SEQ ID NO: 22-24, or a sequence at least 90% or at least 95% identical thereto.

Embodiment 19. The method of Embodiment 18, wherein the circular RNA comprises a nucleic acid sequence of SEQ ID NO: 25, or a sequence at least 90% or at least 95% identical thereto.

Embodiment 20. The method of any one of Embodiments 1-19, wherein the circular RNA is substantially non-immunogenic.

Embodiment 21. The method of Embodiment 20, wherein the circular RNA comprises one or more M-6-methyladenosine (m6A) residues.

Embodiment 22. The method of any one of Embodiment 1-21, wherein the circular RNA comprises from about 200 nucleotides to about 5,000 nucleotides.

Embodiment 23. The method of any one of Embodiments 1-22, wherein the circular RNA comprises an internal ribosome entry site (IRES) operably linked to the protein-coding sequence.

Embodiment 24. The method of any one of Embodiments 1-23, wherein the circular RNA encodes two or more reprogramming factors selected from Oct3/4, Klf4, Sox2, Nanog, Lin28, c-Myc, or L-Myc.

Embodiment 25. The method of any one of Embodiments 1-5, wherein the method comprises contacting the cell with a first circular RNA encoding Oct4 or a fragment or variant thereof, a second circular RNA encoding Sox2 or a fragment or variant thereof, a third circular RNA encoding Klf4 or a fragment or variant thereof, a fourth circular RNA encoding C-Myc or a fragment or variant thereof, a fifth circular RNA encoding Lin28 or a fragment or variant thereof, and a sixth circular RNA encoding Nanog or a fragment or variant thereof.

Embodiment 26. The method of any one of Embodiments 1-5, wherein the method comprises contacting the cells with one or more circular RNAs encoding one or more of a group of reprogramming factors consisting of Oct3/4, Klf4, Sox2, Nanog, Lin28, and c-Myc, or fragments or variants thereof.

Embodiment 27. A method of producing an induced pluripotent stem cell (iPSC), the method comprising contacting a CD34+ cell in suspension with six circular RNAs consisting of a first circular RNA encoding Oct4, a second circular RNA encoding Sox2, a third circular RNA encoding Klf4, a fourth circular RNA encoding C-Myc, a fifth circular RNA encoding Lin28, and a sixth circular RNA encoding Nanog, and maintaining the cell under conditions under which the iPSC is obtained.

Embodiment 28. A method of producing an induced pluripotent stem cell (iPSC), the method comprising contacting a CD34+ cell in suspension with three circular RNAs consisting of a first circular RNA encoding Oct4, a second circular RNA encoding Sox2, and a third circular RNA encoding Klf4, and maintaining the cell under conditions under which the iPSC is obtained.

Embodiment 29. The method of Embodiment 28, wherein the method does not comprise contacting the CD34+ cell with a circular RNA encoding any of Nanog, Lin28, and c-Myc.

Embodiment 30. A method of producing an induced pluripotent stem cell (iPSC), the method comprising contacting a CD34+ cell in suspension with three circular RNAs consisting of a first circular RNA encoding Oct4, a second circular RNA encoding Sox2, and a third circular RNA encoding Klf4, and a fourth circular RNA encoding C-Myc, and maintaining the cell under conditions under which the iPSC is obtained.

Embodiment 31. The method of Embodiment 30, wherein the method does not comprise contacting the CD34+ cell with a circular RNA encoding either of Nanog or Lin28.

Embodiment 32. A method of producing an induced pluripotent stem cell (iPSC), the method comprising contacting a CD34+ cell in suspension with three circular RNAs consisting of a first circular RNA encoding Oct4, a second circular RNA encoding Sox2, and a third circular RNA encoding Klf4, and a fourth circular RNA encoding Nanog, and a fifth circular RNA encoding Lin 28, and maintaining the cell under conditions under which the iPSC is obtained.

Embodiment 33. The method of Embodiment 32, wherein the method does not comprise contacting the CD34+ cell with a circular RNA encoding c-Myc.

Embodiment 34. The method of Embodiment any one of Embodiments 1-33, wherein the cell is not contacted with any factor selected from E3, K3, B18R.

Embodiment 35. The method of Embodiment any one of Embodiments 1-33, wherein the cell is not contacted with any micro RNAs (miRs).

Embodiment 36. The method of Embodiment any one of Embodiments 1-33, wherein the cell is not contacted with one or more factor selected from E3, K3, B18R, one or more micro RNAs (miRs), or a combination thereof.

Embodiment 37. The method of any one of 35-36, wherein the miRs comprise miR302a, miR302b, miR302c, miR302d, and miR367.

Embodiment 38. The method of any one of Embodiments 1-37, wherein the cell is directly contacted with the at least one circular RNA.

Embodiment 39. The method of any one of Embodiments 1-38, wherein the cell is contacted with each of the at least one circular RNA once.

Embodiment 40. The method of any one of Embodiments 1-38, wherein the method comprises contacting the cell with each of the at least one circular RNA two, three, four, or more times.

Embodiment 41. The method of any one of Embodiments 1-38, comprising contacting the cell with each of the at least one circular RNA fewer than four times.

Embodiment 42. The method of any one of Embodiments 1-38, comprising contacting the cell with each of the at least one circular RNA from 2 to 4 times.

Embodiment 43. The method of any one of Embodiments 1-42, wherein the concentration of each of the at least one circular RNAs is at least 3 pg RNA/cell.

Embodiment 44. The method of any one of Embodiments 1-42, wherein the concentration of each of the at least one circular RNAs is from about 5 pg RNA/cell to about 15 pg RNA/cell.

Embodiment 45. The method of any one of Embodiments 1-44, wherein the contacting the cell is performed by electroporation.

Embodiment 46. The method of any one of Embodiments 1-26 or 38-45, wherein the method comprises further contacting the cell with an RNA polynucleotide encoding one or more viral proteins selected from E3, K3, or B18R.

Embodiment 47. The method of Embodiment 46, wherein: (a) the B18R has a sequence of SEQ ID NO: 26, or a sequence at least 90% or at least 95% identical thereto; (b) the E3 has a sequence of SEQ ID NO: 27, or a sequence at least 90% or at least 95% identical thereto; and/or (c) the K3 has a sequence of SEQ ID NO: 28, or a sequence at least 90% or at least 95% identical thereto.

Embodiment 48. The method of any one of Embodiments 1-26 or 38-47, wherein the method comprises further contacting the cell with one or more microRNAs (miRs).

Embodiment 49. The method of Embodiment 48, wherein the miRs are selected from miR302a, miR302b, miR302c, miR302d, and miR367.

Embodiment 50. The method of any one of Embodiments 1-49, wherein the method results in one or more of: (i) an increase in the number of reprogrammed iPSC present at the end of culture compared to a method of producing an iPSC with one or more linear RNAs; and/or (ii) a decrease in cell death at one or more timepoints during reprogramming compared to a method of producing an iPSC with one or more linear RNAs.

Embodiment 51. The method of any one of Embodiments 1-49, wherein the method results in each of: (i) an increase in the number of reprogrammed iPSC present at the end of culture compared to a method of producing an iPSC with one or more linear RNAs; and (ii) a decrease in cell death at one or more timepoints during reprogramming compared to a method of producing an iPSC with one or more linear RNAs.

Embodiment 52. An iPSC produced using the method of any one of Embodiments 1-51.

Embodiment 53. A differentiated cell derived from the iPSC of Embodiment 52.

Embodiment 54. The differentiated cell of Embodiment 53, wherein the differentiated cell is a muscle cell, a neural cell, a cell of the central nervous system, an ocular cell, a chondrocyte, an osteocyte, a tendon cell, a renal cell, a cardiomyocyte, a hepatocyte, an islet cell, a keratinocyte, a T-cell, or a NK-cell.

Embodiment 55. A method for reprogramming and editing the genome of a cell, the method comprising: (i) contacting the cell with a recombinant circular RNA comprising a protein-coding sequence, wherein the protein-coding sequence encodes at least one reprogramming factor, and (ii) contacting the cell with an enzyme capable of editing the DNA or RNA of the cell, or a nucleic acid encoding the same.

Embodiment 56. A method for reprogramming and editing the genome of a cell, the method comprising simultaneously contacting the cell with: (i) a recombinant circular RNA comprising a protein-coding sequence, wherein the protein-coding sequence encodes at least one reprogramming factor, and (ii) an enzyme capable of editing the DNA or RNA of the cell, or a nucleic acid encoding the same.

Embodiment 57. The method of Embodiment 55 or 56, wherein the at least one reprogramming factor is a human or a humanized reprogramming factor.

Embodiment 58. The method of any one of Embodiments 55-57, wherein the at least one reprogramming factor is Oct3/4, and wherein the Oct3/4 has the amino acid sequence of SEQ ID NO: 7, or a sequence at least 90% or at least 95% identical thereto.

Embodiment 59. The method of Embodiment 59, wherein the recombinant circular RNA comprises a nucleic acid sequence of SEQ ID NO: 8, or a sequence at least 90% or at least 95% identical thereto.

Embodiment 60. The method of any one of Embodiments 55-57, wherein the at least one reprogramming factor is Klf4, and wherein the Klf4 has the amino acid sequence of SEQ ID NO: 9 or 10, or a sequence at least 90% or at least 95% identical thereto.

Embodiment 61. The method of Embodiment 60, wherein the recombinant circular RNA comprises a nucleic acid sequence of SEQ ID NO: 11, or a sequence at least 90% or at least 95% identical thereto.

Embodiment 62. The method of any one of Embodiments 55-57, wherein the at least one reprogramming factor is Sox2, and wherein Sox2 has the amino acid sequence of SEQ ID NO: 12, or a sequence at least 90% or at least 95% identical thereto.

Embodiment 63. The method of Embodiment 62, wherein the recombinant circular RNA comprises a nucleic acid sequence of SEQ ID NO: 13, or a sequence at least 90% or at least 95% identical thereto.

Embodiment 64. The method of any one of Embodiments 55-57, wherein the at least one reprogramming factor is Nanog, and wherein the Nanog has the amino acid sequence of SEQ ID NO: 14 or 15, or a sequence at least 90% or at least 95% identical thereto.

Embodiment 65. The method of Embodiment 64, wherein the recombinant circular RNA comprises a nucleic acid sequence of SEQ ID NO: 16, or a sequence at least 90% or at least 95% identical thereto.

Embodiment 66. The method of any one of Embodiments 55-57, wherein the at least one reprogramming factor is Lin28A, and wherein the Lin28A has the amino acid sequence of SEQ ID NO: 17, or a sequence at least 90% or at least 95% identical thereto.

Embodiment 67. The method of Embodiment 66, wherein the recombinant circular RNA comprises a nucleic acid sequence of SEQ ID NO: 18, or a sequence at least 90% or at least 95% identical thereto.

Embodiment 68. The method of any one of Embodiments 55-57, wherein the at least one reprogramming factor is c-Myc, and wherein the c-Myc has the amino acid sequence of SEQ ID NO: 19 or 20, or a sequence at least 90% or at least 95% identical thereto.

Embodiment 69. The method of Embodiment 68, wherein the recombinant circular RNA comprises a nucleic acid sequence of SEQ ID NO: 21, or a sequence at least 90% or at least 95% identical thereto.

Embodiment 70. The method of any one of Embodiments 55-57, wherein the at least one reprogramming factor is L-Myc, and wherein the L-Myc has the amino acid sequence of any one of SEQ ID NO: 22-24, or a sequence at least 90% or at least 95% identical thereto.

Embodiment 71. The method of Embodiment 70, wherein the recombinant circular RNA comprises a nucleic acid sequence of SEQ ID NO: 25, or a sequence at least 90% or at least 95% identical thereto.

Embodiment 72. The method of any one of Embodiments 55-57, wherein the circular RNA is substantially non-immunogenic.

Embodiment 73. The method of Embodiment 72, wherein the circular RNA comprises one or more M-6-methyladenosine (m6A) residues.

Embodiment 74. The method of any one of Embodiment 55-73, wherein the circular RNA comprises from about 200 nucleotides to about 5,000 nucleotides.

Embodiment 75. The method of any one of Embodiments 55-74, wherein the circular RNA comprises an internal ribosome entry site (IRES) operably linked to the protein-coding sequence.

Embodiment 76. The method of any one of Embodiments 55-75, wherein the circular RNA encodes two or more reprogramming factors selected from Oct3/4, Klf4, Sox2, Nanog, Lin28, c-Myc, or L-Myc.

Embodiment 77. The method of Embodiment 55 or 56, wherein the method comprises contacting the cell with a first circular RNA encoding Oct4 or a fragment or variant thereof, a second circular RNA encoding Sox2 or a fragment or variant thereof, a third circular RNA encoding Klf4 or a fragment or variant thereof, a fourth circular RNA encoding C-Myc or a fragment or variant thereof, a fifth circular RNA encoding Lin28 or a fragment or variant thereof, and a sixth circular RNA encoding Nanog or a fragment or variant thereof.

Embodiment 78. The method of Embodiment 55 or 56, wherein the method comprises contacting the cells with one or more circular RNAs encoding Oct3/4, Klf4, Sox2, Nanog, Lin28, c-Myc, and L-Myc, or fragments or variants thereof.

Embodiment 79. The method of Embodiment any one of Embodiments 55-78, wherein the cell is not contacted with any ancillary factors selected from E3, K3, B18R, or any micro RNAs (miRs).

Embodiment 80. The method of any one of Embodiments 55-79, wherein the cell is directly contacted with the at least one circular RNA.

Embodiment 81. The method of any one of Embodiments 55-80, wherein the cell is contacted with each of the at least one circular RNAs once.

Embodiment 82. The method of any one of Embodiments 55-80, wherein the method comprises contacting the cell with each of the at least one of the circular RNAs two, three, four, or more times.

Embodiment 83. The method of any one of Embodiments 55-80, comprising contacting the cell with each of the at least one circular RNA fewer than four times.

Embodiment 84. The method of any one of Embodiments 55-80, comprising contacting the cell with each of the at least one circular RNAs from 2 to 4 times.

Embodiment 85. The method of any one of Embodiments 55-84, wherein the concentration of the at least one circular RNAs is at least 3 pg RNA/cell.

Embodiment 86. The method of any one of Embodiments 55-85, wherein the concentration of the at least one circular RNAs is from about 5 pg RNA/cell to about 15 pg RNA/cell.

Embodiment 87. The method of any one of Embodiments 55-86, wherein the contacting the cell is performed by electroporation.

Embodiment 88. The method of Embodiment 87, wherein the electroporation uses the Neon® electroporation system.

Embodiment 89. The method of any one of Embodiments 55-78 or 80-88, wherein the method comprises contacting the cell with an RNA polynucleotide encoding one or more viral proteins selected from E3, K3, or B18R.

Embodiment 90. The method of Embodiment 89, wherein: (a) the B18R has a sequence of SEQ ID NO: 26, or a sequence at least 90% or at least 95% identical thereto; (b) the E3 has a sequence of SEQ ID NO: 27, or a sequence at least 90% or at least 95% identical thereto; and/or (c) the K3 has a sequence of SEQ ID NO: 28, or a sequence at least 90% or at least 95% identical thereto.

Embodiment 91. The method of any one of Embodiments 55-78 or 80-90, wherein the method comprises contacting the cell with an microRNA (miR) selected from miR302a, miR302b, miR302c, miR302d, and miR367.

Embodiment 92. The method of any one of Embodiments 55-88, wherein the cell is not contacted with any viral proteins selected from E3, K3, B18R, or any micro RNAs (miRs) selected from miR302a, miR302b, miR302c, miR302d, and miR367.

Embodiment 93. The method of any one of Embodiments 55-82, wherein the enzyme is a transcription activator-like effector nuclease (TALEN), an argonaute endonuclease (NgAgo), a structure-guided endonuclease (SGN), an RNA-guided nuclease (RGN), an Adenosine deaminase acting on RNA (ADAR), or modified or truncated variants thereof.

Embodiment 94. The method of Embodiment 93, wherein the RGN is a Cas nuclease selected from Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or a modified or truncated variant thereof.

Embodiment 95. The method of Embodiment 94, wherein the RGN is a Cas9 nuclease, and the Cas9 nuclease is isolated or derived from S. pyogenes or S. aureus.

Embodiment 96. The method of Embodiment 93, wherein the RGN is selected from any one of APG05083.1, APG07433.1, APG07513.1, APG08290.1, APG05459.1, APG04583.1, and APG1688.1, APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, APG07433.1, APG00969, APG03128, APG09748, APG00771, APG02789, APG09106, APG02312, APG07386, APG09980, APG05840, APG05241, APG07280, APG09866, and APG00868.

Embodiment 97. The method of any one of Embodiments 55-96, wherein the method further comprises contacting the cell with a guide RNA, or a nucleic acid encoding the same.

Embodiment 98. The method of any one of Embodiments 55 or 57-97, wherein the cell is contacted with the circular RNA before it is contacted with the enzyme or the nucleic acid encoding the same.

Embodiment 99. The method of any one of Embodiments 55 or 87-97, wherein the cell is contacted with the recombinant circular RNA after it is contacted with the enzyme or the nucleic acid encoding the same.

Embodiment 100. The method of any one of Embodiments 55-99, wherein the cell is contacted with an enzyme capable of editing the DNA or RNA of the cell, or a nucleic acid encoding the same, and a guide RNA, or a nucleic acid encoding the same.

Embodiment 101. The method of Embodiment 100, wherein the enzyme is capable of editing the DNA of the cell and wherein the enzyme and the guide RNA are complexed as a ribonucleoprotein prior to contact with the cell.

Embodiment 102. The method of any one of Embodiments 55-101, wherein the contacting the cell is performed by electroporation.

Embodiment 103. A cell generated by the method of any one of Embodiments 55-102.

Embodiment 104. A method for reprogramming a cell, the method comprising contacting a cell with one or more circular RNAs encoding six reprogramming factors consisting of Oct3/4, Klf4, Sox2, Nanog, Lin28, and c-Myc.

Embodiment 105. The method of Embodiment 104, comprising contacting a cell with six circular RNAs each encoding a reprogramming factor from the group consisting of Oct3/4, Klf4, Sox2, Nanog, Lin28, and c-Myc.

Embodiment 106. The method of Embodiment 104 or 105, wherein any one of the circular RNA or linear RNAs are conjugated to a lipid nanoparticle.

Embodiment 107. The method of Embodiment any one of Embodiments 104-106, wherein the cell is not contacted with one or more factors selected from E3, K3, B18R, or one or more micro RNAs (miRs).

107A. The method of any one of claim 1-51, 55-102, or 104-107, wherein the circular RNA is exogenous to the cell.

Embodiment 108. A cell generated by the method of any one of Embodiments 104-107A.

Embodiment 109. A somatic cell comprising one or more exogenous circular RNAs encoding a reprogramming factor, wherein the reprogramming factor is selected from the group consisting of Oct3/4, Klf4, Sox2, Nanog, Lin28, and c-Myc.

Embodiment 110. The somatic cell of Embodiment 109, wherein the somatic cell comprises one or more exogenous circular RNAs, wherein the one or more circular RNAs each encode a reprogramming factor selected from Oct3/4, Klf4, Sox2, Nanog, Lin28, and c-Myc.

Embodiment 111. The somatic cell of Embodiment 109, wherein the somatic cell comprises six exogenous circular RNAs, wherein each circular RNA encodes one of Oct3/4, Klf4, Sox2, Nanog, Lin28, and c-Myc.

Embodiment 112. The somatic cell of Embodiment 109, wherein the somatic cell comprises five exogenous circular RNAs, wherein each circular RNA encodes one of Oct3/4, Klf4, Sox2, Nanog, and Lin28.

Embodiment 113. The somatic cell of Embodiment 109, wherein the somatic cell comprises four exogenous circular RNAs, wherein each circular RNA encodes one of Oct3/4, Klf4, Sox2, and c-Myc.

Embodiment 114. The somatic cell of Embodiment 109, wherein the somatic cell comprises three exogenous circular RNAs, wherein each circular RNA encodes one of Oct3/4, Klf4, and Sox2.

Embodiment 115. A suspension culture comprising one or more CD34+ cells, wherein the CD34+ cells comprise one or more exogenous circRNAs encoding a reprogramming factor.

Embodiment 116. The suspension culture of Embodiment 115, wherein the CD34+ cells comprise six exogenous circRNAs each encoding one reprogramming factor selected from the group consisting of Oct3/4, Klf4, Sox2, Nanog, Lin28, and c-Myc.

Embodiment 117. The suspension culture of Embodiment 115 or 116, wherein the CD34+ cell does not comprise an exogenous nucleic acid encoding an ancillary factor selected from E3, K3, B18R, or a micro RNAs (miRs).

Embodiment 118. A composition comprising one or more circular RNAs encoding the reprogramming factors consisting of Oct3/4, Klf4, Sox2, Nanog, Lin28, and c-Myc.

Embodiment 119. A composition comprising two or more circular RNAs encoding the reprogramming factors consisting of Oct3/4, Klf4, Sox2, Nanog, Lin28, and c-Myc.

Embodiment 120. A composition comprising six circular RNAs each encoding one of the reprogramming factors consisting of Oct3/4, Klf4, Sox2, Nanog, Lin28, and c-Myc.

Embodiment 121. A composition comprising five circular RNAs each encoding one of the reprogramming factors consisting of Oct3/4, Klf4, Sox2, Nanog, and Lin28.

Embodiment 122. A composition comprising four circular RNAs each encoding one of the reprogramming factors consisting of Oct3/4, Klf4, Sox2, and c-Myc.

Embodiment 123. A composition comprising three circular RNAs each encoding one of the reprogramming factors consisting of Oct3/4, Klf4, and Sox2.

Embodiment 124. A kit comprising the composition of any one of Embodiments 118-123.

Embodiment 125. A cell comprising the composition of any one of Embodiments 118-123.

Embodiment 126. The cell of Embodiment 125, wherein the cell is a eukaryotic cell.

Embodiment 127. The cell of Embodiment 126, wherein the cell is a mammalian cell.

Embodiment 128. The cell of Embodiment 127, wherein the cell is a human cell.

Embodiment 129. The cell of any one of Embodiments 125-128, wherein the cell is a CD34+ cell, a T cell, or an NK cell.

Embodiment 130. A CD34+ cell comprising one or more circular RNAs encoding one or more reprogramming factors selected from Oct3/4, Klf4, Sox2, Nanog, Lin28, and either one of c-Myc, or L-Myc.

Embodiment 131. The CD34+ cell of Embodiment 130, wherein the reprogramming factors consist of all six of Oct3/4, Klf4, Sox2, Nanog, Lin28, and c-Myc.

Embodiment 132. The CD34+ cell of Embodiment 130, wherein the reprogramming factors consist of all six of Oct3/4, Klf4, Sox2, Nanog, Lin28, and L-Myc.

Embodiment 133. The CD34+ cell of Embodiment 130, wherein the reprogramming factors consist of Oct3/4, Klf4, Sox2, Nanog, and Lin28.

Embodiment 134. The CD34+ cell of Embodiment 130, wherein the reprogramming factors consist of Oct3/4, Klf4, Sox2, and c-Myc.

Embodiment 135. The CD34+ cell of Embodiment 130, wherein the reprogramming factors consist of Oct3/4, Klf4, and Sox2.

Embodiment 136. The CD34+ cell of any one of Embodiments 130-135, wherein the cells exhibit at least one stemness marker selected from SSEA-3, SSEA-4, TRA-1-60, TRA-1-81, TRA-2-49/6E, Alkaline phosphatase, Sox2, E-cadherin, UTF-1, Oct4, Rex1, Nanog, or a combination thereof.

Embodiment 137. The CD34+ cell of any one of Embodiments 130-136, wherein the one or more circular RNAs is exogenous to the cells.

Embodiment 138. The CD34+ cell of any one of Embodiments 130-137, further comprising one or more genetic modifications.

Embodiment 139. The CD34+ cell of Embodiment 138, wherein the one or more genetic modification comprises a gene knockout.

Embodiment 140. The CD34+ cell of Embodiment 138 or 139, wherein the one or more genetic modification comprises a gene knock-in.

Embodiment 141. An induced pluripotent stem cell (iPSC) derived from the CD34+ cell of any one of Embodiments 130-140.

Embodiment 142. The iPSC of Embodiment 141, wherein the cell is hypoimmunogenic.

Embodiment 143. A differentiated cell generated from the iPSC of Embodiment 141 or 142.

Embodiment 144. A method of treating a disease or condition comprising administering to a subject in need thereof the iPSC of Embodiment 141 or 142 or the differentiated cell of Embodiment 126.

Embodiment 145. A method of transdifferentiating a somatic cell comprising contacting the cell with one or more exogenous circular RNAs.

Embodiment 146. A transdifferentiated cell produced by the method of Embodiment 145.

Embodiment 147. A method of differentiating a cell from an induced pluripotent stem cell (iPSC) comprising contacting the iPSC with one or more circular RNAs.

Embodiment 148. A differentiated cell produced by the method of Embodiment 147.

### EXAMPLES

The following examples, which are included herein for illustration purposes only, are not intended to be limiting.

### Example 1: Circular RNA Reprogramming of CD34+ Cells in Suspension

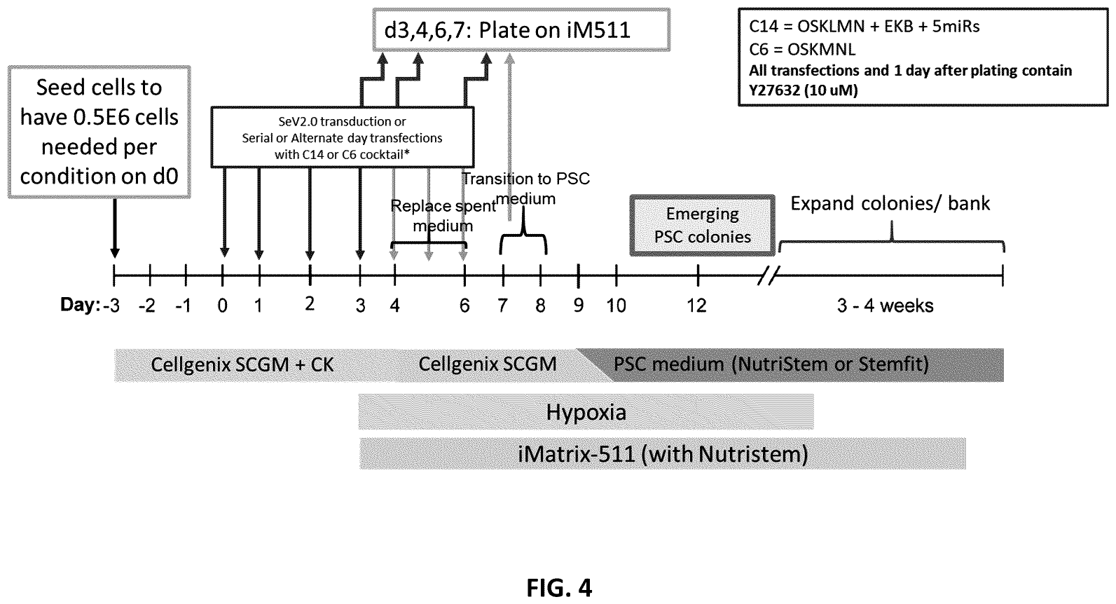

Experiments were performed to assess the feasibility of reprogramming CD34+ cells in suspension. A schematic of the timeline and process for the reprogramming experiment is provided in FIG. 4. Exemplary sequences of circularized RNAs are provided in SEQ ID NOs: 8, 11, 13, 16, 18, and 21 and detailed below in Table 6.

TABLE 6
Exemplary Circularized RNAs

| Reference ID | Encoded Gene | SEQ ID NO: |
|---|---|---|
| OCT4_circRNA | OCT4 | 8 |
| SOX2_circRNA | SOX2 | 13 |
| LIN28_circRNA | LIN28A | 18 |
| NANOG_circRNA | NANOG | 16 |
| KLF4_circRNA | KLF4 | 11 |
| cMYC_circRNA | cMYC | 21 |

Briefly, cord blood-derived CD34+ cells were expanded for 3 days (d-3 to do) in SCGM media (Cellgenix) containing 5 cytokines—SCF, FLT3L, TPO, IL-3, and IL-6 (each at 100 ng/ml). On Day 0, 0.5×10⁶ cells per condition were pelleted, washed, and resuspended in 100-120 μL of buffer for transfection by electroporation. Cells were electroporated with a Neon® electroporator set at 1600V, 10 ms pulse width, 3 pulses. RNA cocktails (C14 and C6) were delivered to CD34 cells at 2 concentrations: 5.2 pg/cell and 13 pg/cell. These concentrations were chosen based on previous experiments which used lower RNA/cell ratios (2.6 pg/cell) and were unsuccessful in CD34+ cell reprogramming (data not shown). The RNA cocktails used in this experiment comprise the following components:
(a) C14 cocktail—6 circRNAs separately encoding OCT4, SOX2, KLF4, cMYC, NANOG and LIN28A (OSKMNL)+linear RNA encoding E3/K3/B18R (EKB)+5 miRs (miR302a, -b, -c, -d and miR367)
(b) C6 cocktail=6 circRNAs separately encoding OCT4, SOX2, KLF4, cMYC, NANOG and LIN28A (OSKMNL).

The stoichiometry of the 6 reprogramming factors OSKMNL is 3:1:1:1:1:1. Transfection frequencies and RNA amounts delivered in each of the cocktails are shown in the Table 7.

TABLE 7
Experimental details for CD34+ cell reprogramming

| | CircRNA | RNA (pg)/CD34 | Frequency of | # of |
|---|---|---|---|---|
| Group # | Cocktail | cell/transfection | transfection | transfections |
| 1 | C14 | 5.2 | 2 Alternate | 2 (d 0, d 2) |
| 2 | C14 | 5.2 | 4 Alternate | 4 (d 0, d 2, d 4, d 6) |
| 3 | C14 | 5.2 | 4 Daily | 4 (d 0-d 3) |
| 4 | C14 | 5.2 | 6 Daily | 6 (d 0-d 5 |
| 5 | C14 | 13 | 2 Alternate | 2 (d 0, d 2) |
| 6 | C14 | 13 | 4 Alternate | 4 (d 0, d 2, d 4, d 6) |
| 7 | C14 | 13 | 4 Daily | 4 (d 0-d 3) |
| 8 | C14 | 13 | 6 Daily | 6 (d 0-d 5) |
| 9 | C6 | 3.2 | 6 Daily | 6 (d 0-d 5) |
| 10 | C6 | 8 | 6 Daily | 6 (d 0-d 5) |
| 11 | SeV | — | Single | — |
| | (CytoTune) | | transduction | |
| 12 | Mock + SeV | — | Single | 2 (d 0, d 2) |
| | | | transduction + | |
| | | | 2 alternate Txs | |
| 13 | Mock (4Alt) | NA | 4 Alternate | 4 |
| 14 | Mock (6 daily) | NA | 6 Daily | 6 |
| 15 | CD34 alone | NA | NA | NA |

CD34+ cells (0.5×10⁶) transduced on do with Sendai virus (Cytotune 2.0 kit) expressing OSKM were used as a positive control. Negative controls included mock transfections (with RNase and DNase-free water) done simultaneously with other transfections (once daily for 6 days (Group 14 in Table 7) or once every alternate day for 4 days (Group 13 in Table 7)) as well as non-transfected CD34 cells. All controls were performed with 0.5×10⁶ CD34+ cells.

Cells were resuspended in complete SCGM media containing 10 UM of the ROCK inhibitor Y27632 after every transfection and seeded on 24-well non-adherent plates. On the day after the last transfection, cell count and viability were determined for each condition including the controls (SeV, mock, and CD34+ only cells). All cells were plated in iMatrix-511 coated 12-well tissue culture-treated plate with complete SCGM media (with cytokines) and 10 μM Y27632. Plated cells were grown in 5% 02 (hypoxia incubator).

On days 4 to 6, half of the media (1 ml) from each well was replaced with 1 ml SCGM media without cytokines. Replacement media for Day 4 comprised 10 μM Y27632. Replacement media on the remaining days did not comprise 10 μM Y27632. On Day 7, half of the media (1 ml) from each well was replaced with 1 ml of PSC media (Nutristem hPSC-XF for cells grown on iM511 or Stemfit media for cells grown on Vtn). From day 8 onwards, until iPSC colonies were ready to be picked, all of the media in each well was replaced with 2 ml PSC media.

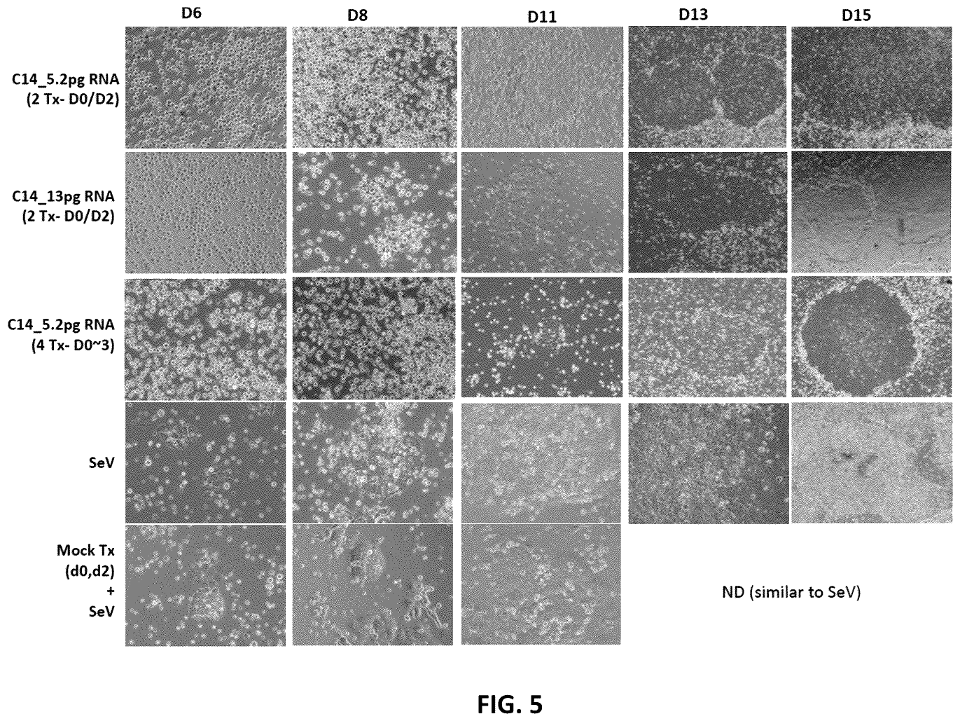

Small clumps resembling iPSC-like colonies were first observed by d6-d7 (for SeV) and d8 (for C14). By day 11, cell clusters were observed in three of the circRNA conditions (top three rows). These clusters continued to proliferate and grow in size and exhibited iPSC morphology. See FIG. 5.

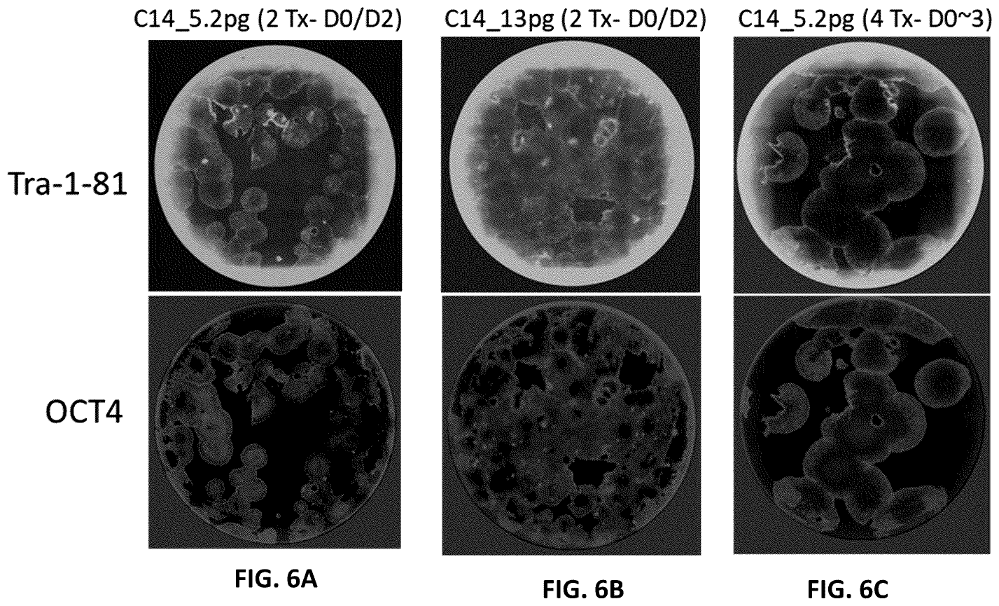

On day 22-23 of reprogramming, cultures were fixed and stained with pluripotency markers Tra-1-81 and OCT4, and whole-well images were scanned using IncuCyte. The iPSC colonies showed positive staining for the pluripotency markers Tra-1-81+ and Oct4+. Cultures which underwent 2 transfections (performed on day 0 and day 2), using either 5.2 pg (Group 1) or 13 pg (Group 5) of the C14 cocktail (FIG. 6A and FIG. 6B) showed large areas of Tra-1-81/Oct4-double positive cells. Four daily transfections of 5.2 pg (Group 3) of the C14 cocktail (FIG. 6C) also produced iPSC colonies that were positive for TRA-1-81 ad OCT4.

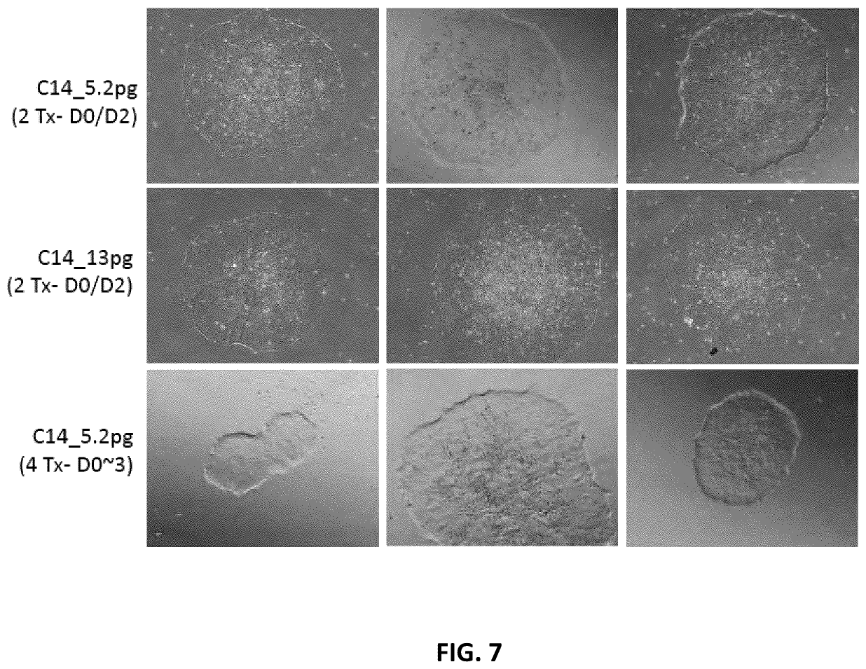

Representative phase contrast images of circRNA-reprogrammed iPSC clones at passage 1 are shown in FIG. 7. These iPSC clones derived from circRNA reprogrammed CD34 cells were able to be picked and expanded in culture.

These results demonstrate reprogramming of CD34+ cells in suspension using circRNAs encoding reprogramming factors. The C14 cocktail successfully reprogrammed CD34 cell by either 1) transfection on day 0 and day 2, or 2) transfecting on days 1, 2, 3 and 4. Reprogramming efficiencies were similar to SeV reprogramming (group 11) further demonstrating the robustness of circRNA reprogramming. Mock electroporation (×2) followed by SeV reprogramming, while resulting in large amount of cell death, still gave rise to iPSC colonies. This suggests that the electroporation procedure itself is not prohibitive for the initiation and progression of iPSC reprogramming.

### Example 2: Simultaneous Reprogramming and Gene Editing of Fibroblasts

Experiments were performed using fibroblasts (HDFs) to demonstrate simultaneous reprogramming (with circRNAs) and editing of the B2M gene to generate B2M−/− iPSCs.

Two protocols were compared: (1) a 1-step protocol illustrating simultaneous reprogramming and editing of HDFs, and (2) a 2-step protocol illustrating sequential editing and reprogramming of HDFs.

For the 1-step protocol, HDFs were reprogrammed with circRNA following previously optimized circRNA reprogramming protocol—i.e., 2 transfections performed on day 0 and 2 using RNAiMAX transfection reagent. The C14 reprogramming cocktail was used, comprising 6 circRNAs encoding the reprogramming factors OCT4, SOX2, KLF4, cMYC, NANOG and LIN28A (OSKLMN)+linear mRNAs encoding E3/K3/B18R+5 microRNAs (miR302a, -b, -c, -d and miR367). An mRNA polynucleotide encoding the LEG14 RNA guided nuclease (Life Edit Therapeutics, Morrisville, NC) and an sgRNA targeting the B2M gene (Life Edit Therapeutics, Morrisville, NC) were co-transfected with the circRNA reprogramming cocktail. The following three conditions were tested:
(a) No co-transfection of Nuclease/sgRNA
(b) Nuclease/sgRNA co-transfected on day 0 (RE1)
(c) Nuclease/sgRNA co-transfected on day 0 and 2 (RE2)

For each of RE1 and RE2, three different densities of HDFs (25K, 50K and 75K per well) were simultaneously reprogrammed and edited.

For the 2-step protocol, HDFs were first transfected with an RNP complex comprising the RNA-guided nuclease and the B2M-targeting sgRNA using a Neon® electroporator. 3 different settings were tested (see Table 9). Electroporated HDFs were then plated in culture to reach optimal confluency (˜6 days) and seeded in 6-well plates for circRNA reprogramming according to the same procedures used for the 1-step protocol.

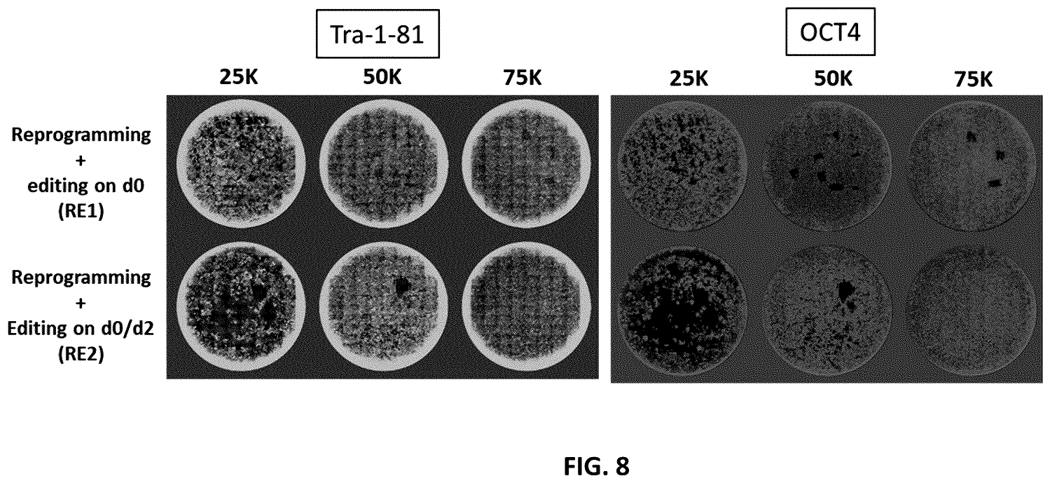

On Day 21 of reprogramming, cultures were fixed and stained with pluripotency markers Tra-1-81 and OCT4, and whole-well images were scanned using IncuCyte. iPSC colonies showed positive staining for Tra-1-81− and Oct4. FIG. 8 shows the staining results for the 1-step protocol. FIG. 10 shows the staining results for the 2-step protocol.

Editing efficiencies for the 1-step (simultaneous) and 2-step (sequential) protocols were measured by the % reads that showed an indel at the region around the sgRNA target site (exon 1 of B2M gene). Editing efficiency was determined by PCR amplification of the region of interest and sequencing the resulting amplicons using Ilumina's Mi-Seq NGS system. Results for the 1-step protocol are shown in Table 8. A 100% editing efficiency was observed when cells were edited twice (RE2) as all reads showed a diversity of indels.

TABLE 8
Editing efficiency of 1-step protocol

| Condition | HDF seeding density | Editing efficiency |
|---|---|---|
| Editing mRNA co- | 25k | 52.54% |
| transfected on D 0 (RE1) | 50k | 49.88% |
| | 75k | 69.11% |
| Editing mRNA co- | 25k | 100% |
| transfected on D 0 and D 2 | 50k | 100% |
| (RE2) | 75k | 100% |

The B2M alleles of 2 iPSC clones derived from 1-step simultaneous reprogramming and editing protocol were further analyzed. Results show that the editing protocol resulted in both heterozygous clones and homozygous clones (data not shown).

Results from the 2-step protocol are shown in Table 9. About 3×10⁵ HDFs were electroporated with a ribonucleoprotein (RNP) complex comprising the RNA-guided nuclease and sgRNA targeting the B2M gene. Edited cells were expanded in culture and seeded at 3 different densities (25K, 50K and 75K) on 6 well plates for reprogramming with the C14 circRNA cocktail. Editing efficiency was determined in 16 different physically separated clones (for the 3 different Neon® parameters) that were subcloned for lysis and PCR amplification of the region of interest. Amplicon reads were sequenced using Ilumina's Mi-Seq NGS system. As shown in Table 9, all three electroporation parameters conditions resulted in high editing efficiencies of the B2M gene.

TABLE 9
Editing efficiency of 2-step protocol

| | Conditions | Editing efficiency |
|---|---|---|
| Control | 75k HDF seeding | 0 |
| | density | |
| (2-step) Transfect HDFs | 1 | 99.3% |
| with RNP using Neon ® | 2 | 99.6% |
| prior to circRNA | 3 | 100% |
| reprogramming | | |

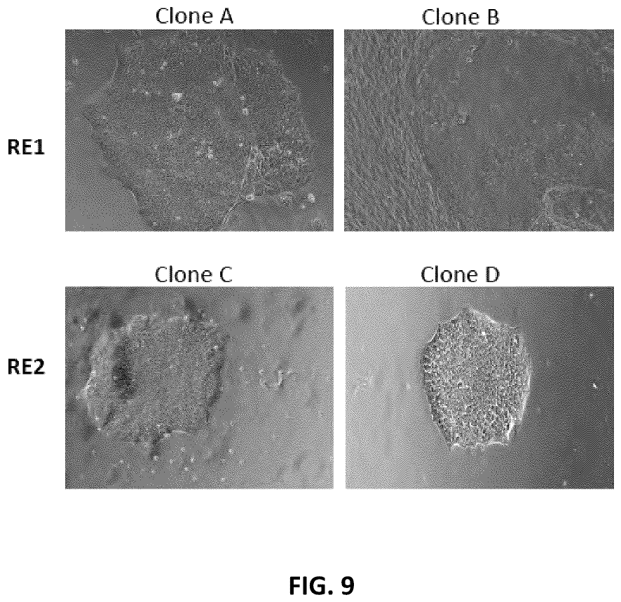

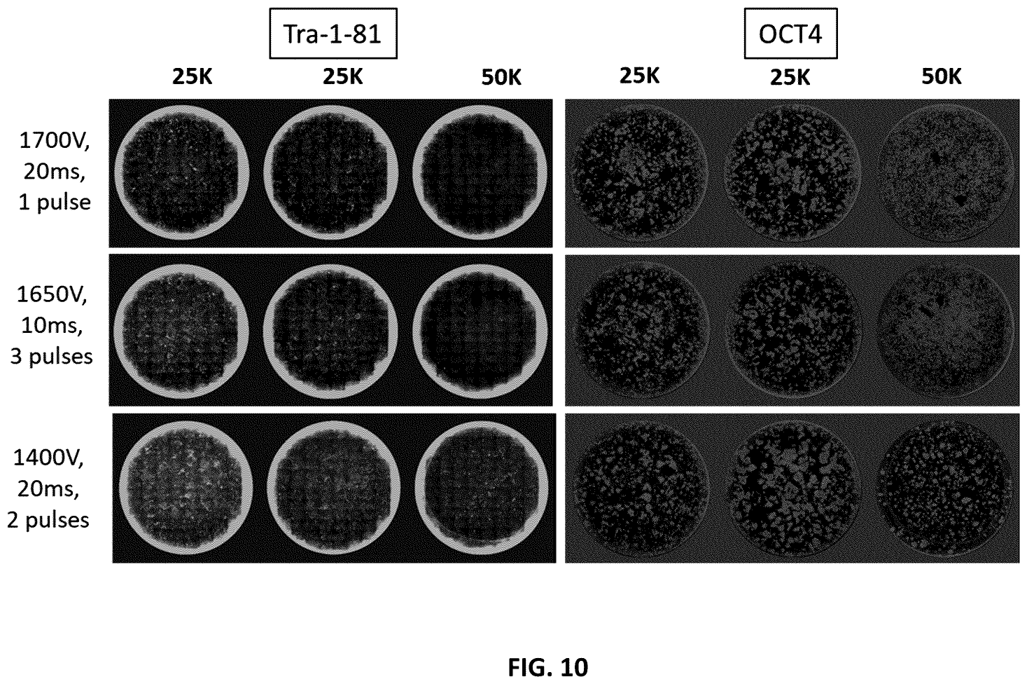

FIG. 9 provides representative images of iPSC clones derived from 1-step simultaneous reprogramming and editing protocol. These clones exhibit typical iPSC morphology and can be expanded in culture. Clones derived from the RE1 protocol (one delivery of editing factors) are shown on the top row. Clones derived from the RE2 protocol (2 deliveries of editing factors) are shown on the bottom row.

These data demonstrate that both the 1-step (simultaneous) and 2-step (sequential) editing protocols successfully generated edited (B2M−/− or B2M−/+) iPSCs. The 1-step protocol represents the true simultaneous reprogramming and editing. One transfection of the editing factors (RNA-guided nuclease+sgRNA) resulted in 50˜70% editing efficiency, and two transfections of the editing factors resulted in ˜100% editing efficiency. The 2-step protocol also resulted in very high editing efficiencies of ˜100%.

### Example 3: CircRNA Reprogramming of CD34+ Cells in Suspension without Ancillary Factors

Experiments were performed to test different transfection regimens for the C6 circRNA cocktail reprogramming of CD34+ cells in suspension. This cocktail contains only circRNAs encoding the reprogramming factors OCT4, SOX2, KLF4, cMYC, LIN28A, and NANOG (OSKMLN). The goal of these experiments was to demonstrate feasibility of reprogramming suspension cells and to further demonstrate that E3/K3/B18R and microRNAs are not required when reprogramming with circRNA cocktails.

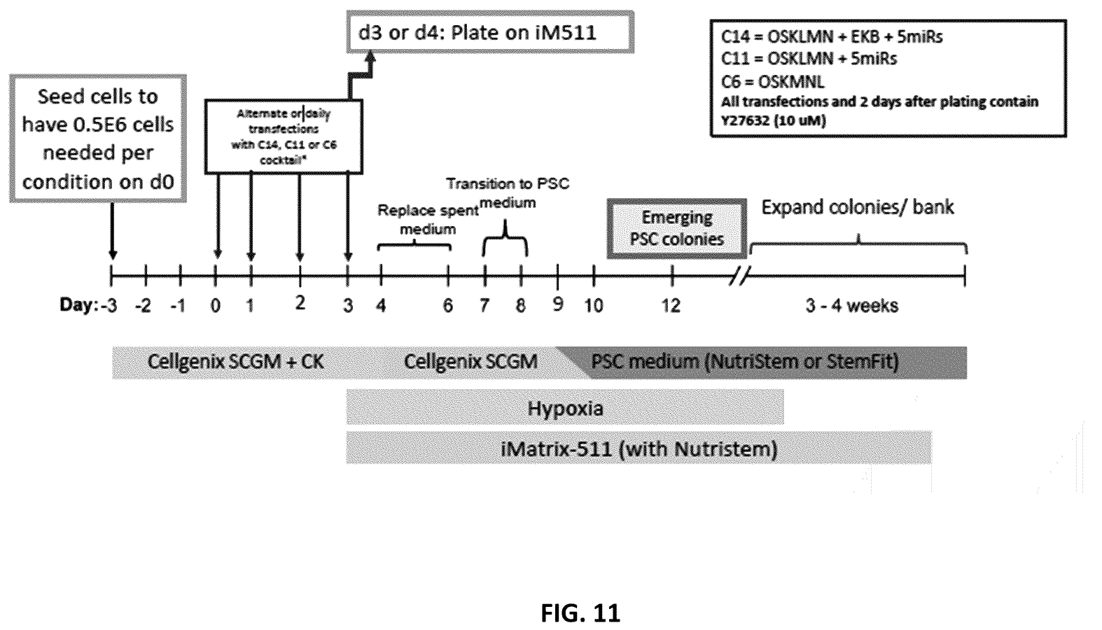

FIG. 11 provides a schematic of the timing and experimental design for these experiments. Table 10 provides the experimental details for each of the conditions tested. In addition to the C14 and C6 cocktails, the C11 cocktail (comprising 6 circRNAs encoding the OSKMLN reprogramming factors plus 5 microRNAs) was also tested.

TABLE 10
Experimental groups for reprogramming conditions

| | circRNA | | | RNA |
|---|---|---|---|---|
| Group # | cocktail | Tx frequency | # of Tx | (pg)/cell/Tx |
| 1 | C14 | Alternate | 2 (d 0, d 2) | 5.2 |
| 2 | C14 | Alternate | 2 (d 0, d 2) | 13 |
| 3 | C6 | Alternate | 2 (d 0, d 2) | 3.2 |
| 4 | C6 | Alternate | 2 (d 0, d 2) | 8 |
| 5 | C6 | Daily | 4 (d 0, 1, 2, 3) | 3.2 |
| 6 | C6 | Daily | 4 (d 0, 1, 2, 3) | 8 |
| 7 | C11 | Alternate | 2 (d 0, d 2) | 4 |
| 8 | C11 | Alternate | 2 (d 0, d 2) | 10 |
| 9 | Mock | Alternate | 2 (d 0, d 2) | — |
| 10 | CD34 | — | 2 (d 0, d 2) | — |

Cord blood-derived CD34+ cells were reprogrammed to iPSCs by electroporating cells with one of the following cocktails:
(a) C14 cocktail—circRNA, separately encoding the OCT4, SOX2, KLF4, cMYC, NANOG and LIN28A (OSKMNL) reprogramming factors+linear RNA encoding E3/K3/B18R+5 miRs (miR302a, -b, -c, -d and miR367);
(b) C11 cocktail—6 circRNAs separately encoding the OCT4, SOX2, KLF4, cMYC, NANOG and LIN28A (OSKMNL) reprogramming factors+5 miRs (miR302a, -b, -c, -d and miR367); or
(c) C6 cocktail—circRNA, separately encoding OCT4, SOX2, KLF4, cMYC, NANOG and LIN28A (OSKMNL)

The stoichiometry of the 6 reprogramming factors OSKMNL was 3:1:1:1:1:1.

Briefly, cord blood-derived CD34+ cells were expanded for 3 days (d −3 to d0) in SCGM media (Cellgenix) containing 5 cytokines-SCF, FLT3L, TPO, IL3 and IL6 (each at 100 ng/ml). On Day 0, 0.5×10⁶ cells per condition were pelleted, washed, and resuspended in 100-120 μL of buffer for transfection by electroporation. Cells were electroporated with a Neon® electroporator set at 1600V, 10 ms pulse width, 3 pulses, in order to introduce the RNA Cocktails. Negative controls for this experiment included mock transfections (with Rnase, Dnase-free water) performed once daily for 4 days, as well as non-transfected CD34+ cells. Both negative controls were performed with 0.5×10⁶ CD34+ cells. Cells were resuspended in complete SCGM media containing 10 μM Y27632 after every transfection and seeded on 24-well non-adherent plates.

One the day after the last transfection, cell counts and viability were determined for each condition including the controls (mock and non-transfected CD34+ cells). All cells were plated in iMatrix-511 coated 12-well tissue culture-treated plate with complete SCGM media (with cytokines) and 10 μM Y27632. Cells were grown in 5% O₂ (hypoxia incubator).

On Days 4 to 5, half of the media (1 ml) from each well was replaced with 1 ml SCGM media without cytokines+10 μM Y27632 media. On Day 6, half of the media (1 ml) from each well was replaced with 1 ml SCGM media without cytokines and without 10 μM Y27632 media. On Day 7, half of the media (1 ml) from each well was replaced with 1 ml of PSC media (Nutristem hPSC-XF for cells grown on iM511 or Stem fit media for cells grown on Vtn). From Day 8 onwards, until iPSC colonies were ready to be picked, all of the media in each well was replaced with 2 ml PSC media.

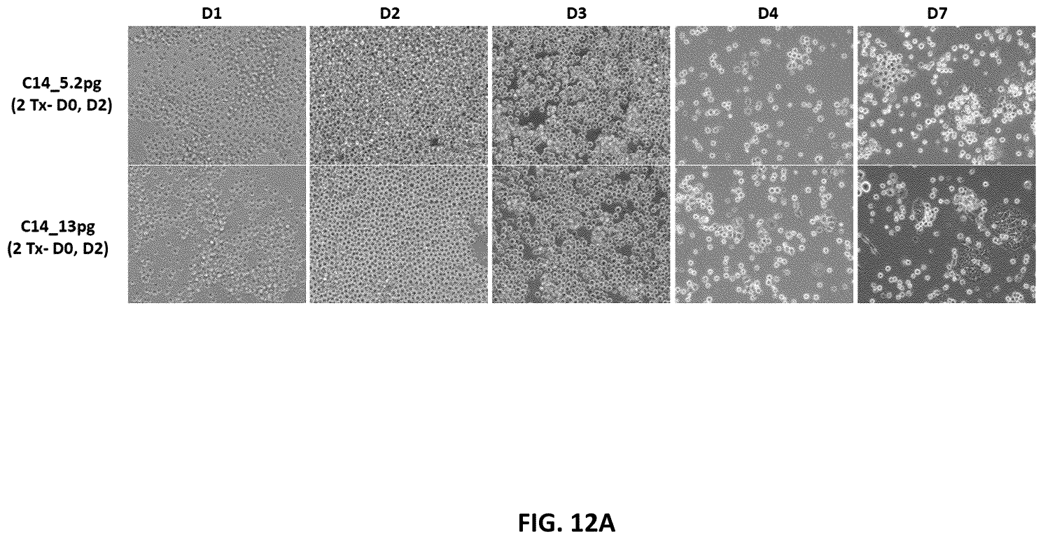

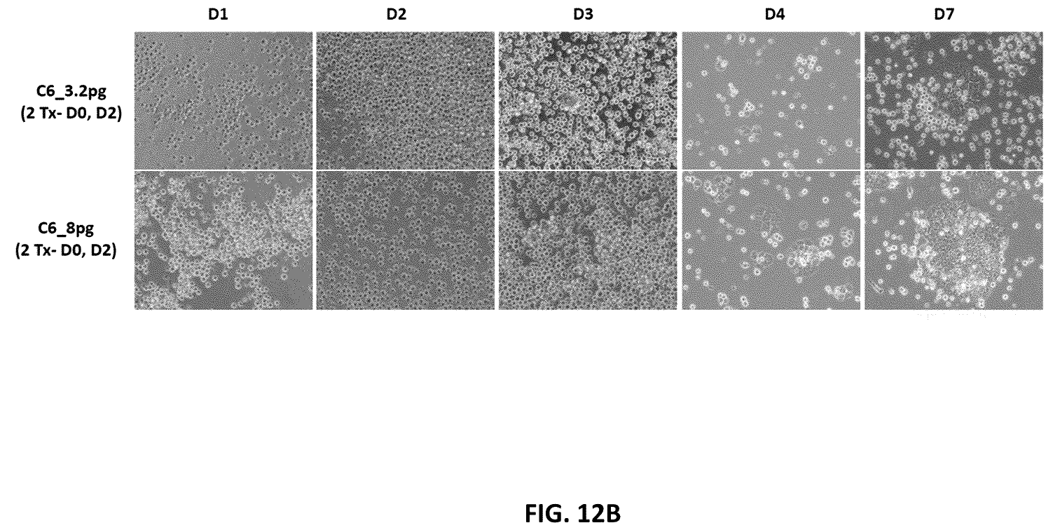

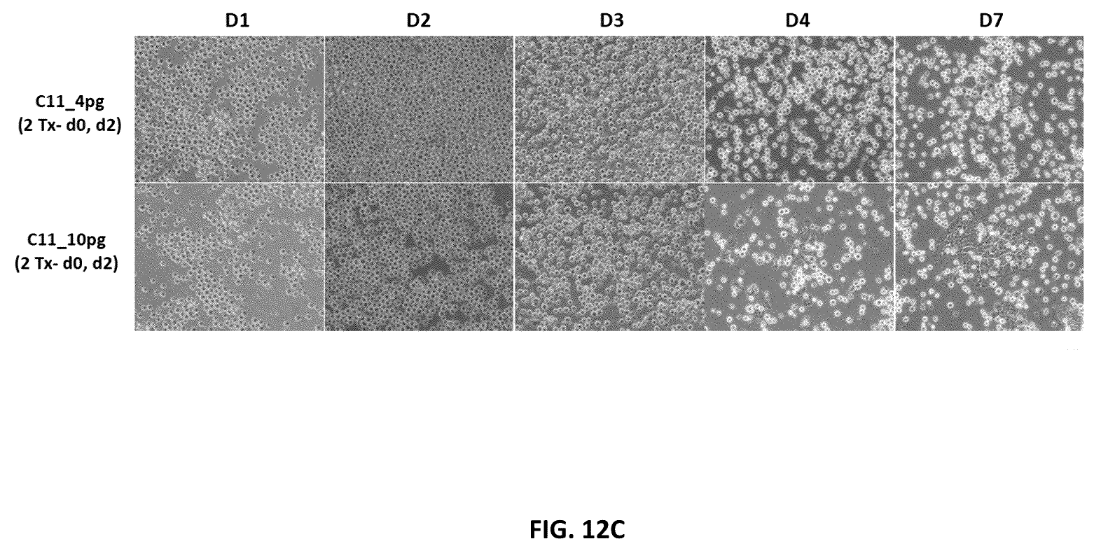

FIG. 12A-FIG. 12C show the morphological progression of CD34+ cell reprogramming using C14, C6, and C11 circRNA cocktails. On day 4, small, attached cell clusters were observed from most conditions. On day 7, early iPSC-like colonies emerged from all conditions. These clusters continued to proliferate and grow in size and exhibited iPSC morphology.

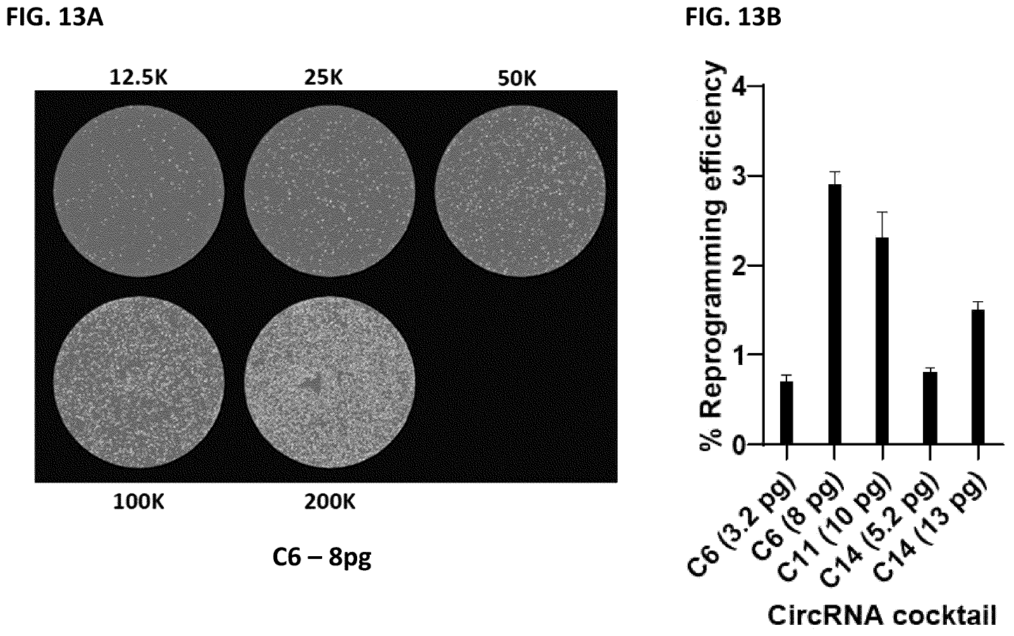

FIG. 13A provides a representative image of a 6-well plate with iPSC colonies reprogrammed with the C6 (8 pg) reprogramming cocktail that were segmented and identified using the object count feature in Incucyte's analysis module. FIG. 13B provides a comparison of the reprogramming efficiencies observed early (d8-d10) during reprogramming with different circRNA cocktails.

All conditions tested (C14, C11, C6 and C14+ editing) yielded iPSC colonies from CD34+ cells by day 4˜7. Both transfection schedules (2 transfections on alternate days (FIG. 12) and 4 daily transfections (data not shown)) resulted in reprogramming and the generation of iPSC. Among these conditions, C6 at 8 pg (with 2 alternate day transfections) yielded maximum number of iPSC colonies compared to all other conditions (See FIG. 13B). Colonies were observed on day 3 even with low seeding densities (as low as 12.5K cells). The C6 cocktail at 8 pg resulted in a reprogramming efficiency of about ˜3%.

### Example 4: Optimizing RNA Electroporation in CD34+ Cells

Experiments were performed to optimize the electroporation protocols for CD34+ cells. Transfection of linear mRNA vs. circRNA RNA was tested, as well as a comparison of Neon® electroporation (100 μL tip volume) and Lonza Amaxa nucleofector (20 μL volume)

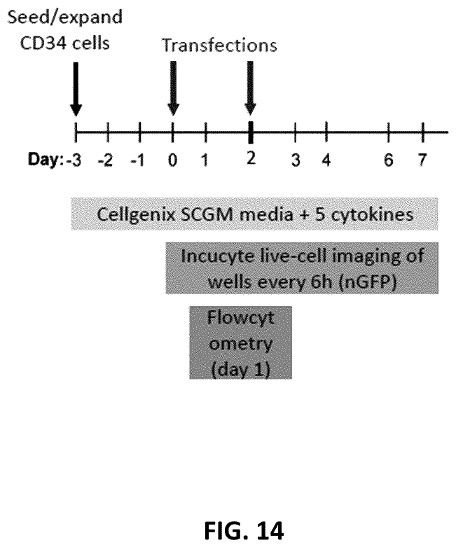

A schematic of the experimental methods is shown in FIG. 14. Briefly, cord blood-derived CD34+ cells were thawed and expanded for 3 days (d-3 to d0) in SCGM media (Cellgenix) containing 5 cytokines-SCF, FLT3L, TPO, IL-3 and IL-6 (each at 100 ng/ml). On day 0, the CD34+ cells were collected, washed, and resuspended in buffer suitable for electroporation using the Neon® system or Lonza's Nucleofactor.

Linear non-modified mRNA (TriLink) or circRNA (non-modified) encoding nGFP (0.25 or 0.5 ug) were electroporated using the Neon® or Lonza electroporation systems. Transfections were performed twice, on days 0 and 2. For Neon®-based transfection, 1×10⁶ cells were washed and resuspended in Buffer T and electroporated with Neon®'s 100 μl tip at 1600V, 10 ms pulse width, with 3 pulses. For Lonza-based transfection, 1×10⁶ cells were washed and resuspended in 20 μl of P3 nucleofector solution with the supplement (4.5:1 ratio) as per the manufacturer's instructions. The Lonza recommended program E0-100 for suspension cells was used to electroporate cells in a 16-well cuvette. Cells were immediately transferred to non-adherent plates containing complete SCGM media.

100,000 cells were harvested on days 1, 2, 3, 4, and 5 post-electroporation and fixed to analyze protein expression. nGFP protein expression was also measured in live cells in suspension using the Incucyte real time imaging platform as well as by flow cytometry after first transfection (to measure nGFP expression at cellular level)

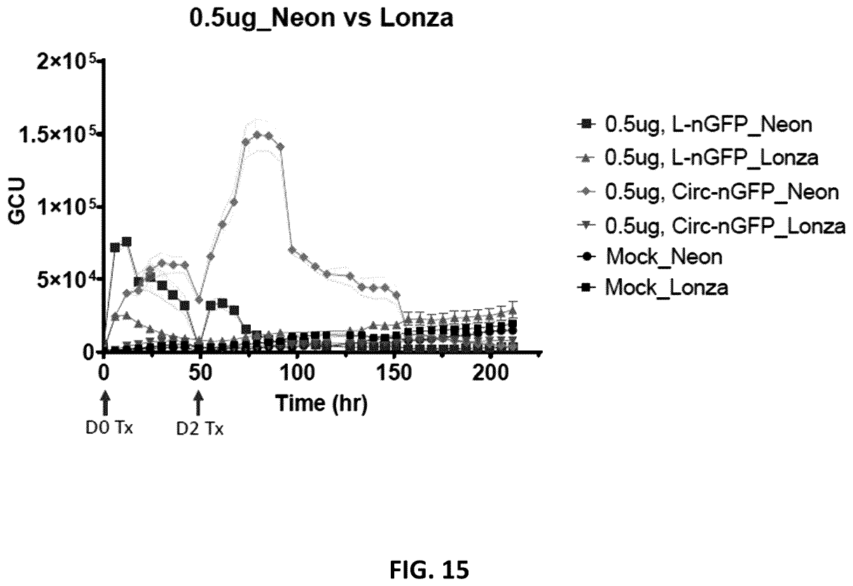

FIG. 15 shows the kinetics of nGFP protein expression as determined by IncuCyte imaging. nGFP protein levels were measured by Incucyte every 6 hours. RNA (linear or circular) transfected with the Neon® electroporator, but not the Lonza Amaxa nucleofector, produced a significant peak of nGFP protein expression following the first transfection on day 0. After the second transfection on day 2, only circRNA transfected with the Neon® electroporator produced a large peak of nGFP protein expression. Linear mRNA transfected with the Neon® electroporator produced a very small peak after the 2^{nd} transfection. Transfection of either linear mRNA or circRNA using Lonza Amaxa nucleofector did not produce any significant nGFP protein expression.

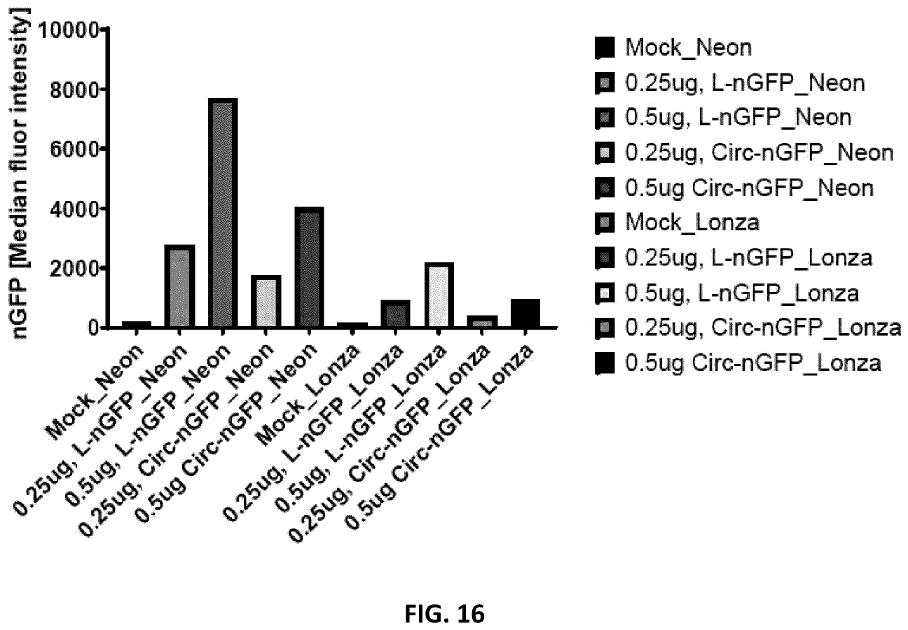

FIG. 16 illustrates flow cytometry analysis of nGFP protein levels one day after the first transfection. RNA transfected with the Neon® electroporator resulted in higher nGFP protein levels than RNA transfected with the Lonza Amaxa nucleofector.

These data demonstrate that, at least under these conditions, electroporation of cord blood-derived CD34+ cells using the Neon® system is more efficient in delivering both linear mRNA and circRNA than electroporation with Lonza's 4D nucleoporator. Upon 2 transfections using the Neon® system, circRNA resulted in a much higher level of nGFP protein than linear mRNA (FIG. 15).

### Example 5: Effects of RNase R Treatment on circRNA Synthesis as Determined by Reporter Protein Expression

Experiments were performed to determine whether RNaseR treatment (as a part of the circRNA production process) had any effect on downstream protein expression and/or cell viability.

Two cell types were used—HDFs and CD34+ cells. A single transfection with circRNA encoding nGFP with or without RNaseR treatment was performed. nGFP coding linear mRNA was used as a reference. Transfection methods and experimental parameters are outlined in Table 11. Readouts for nGFP expression were performed by Incucyte (every 6h) and flow cytometry (every 24h, days 1-5).

TABLE 11
Transfection method and format

| Cell type | # cells/transfection | RNA amount | Transfection Method |
|---|---|---|---|
| HDF | 50k (per 24-well) | 50 ng | RNAiMAX reagent |
| CD34+ | 500k (per 6-well) | 250 ng | Neon ® (100 μL tip) |

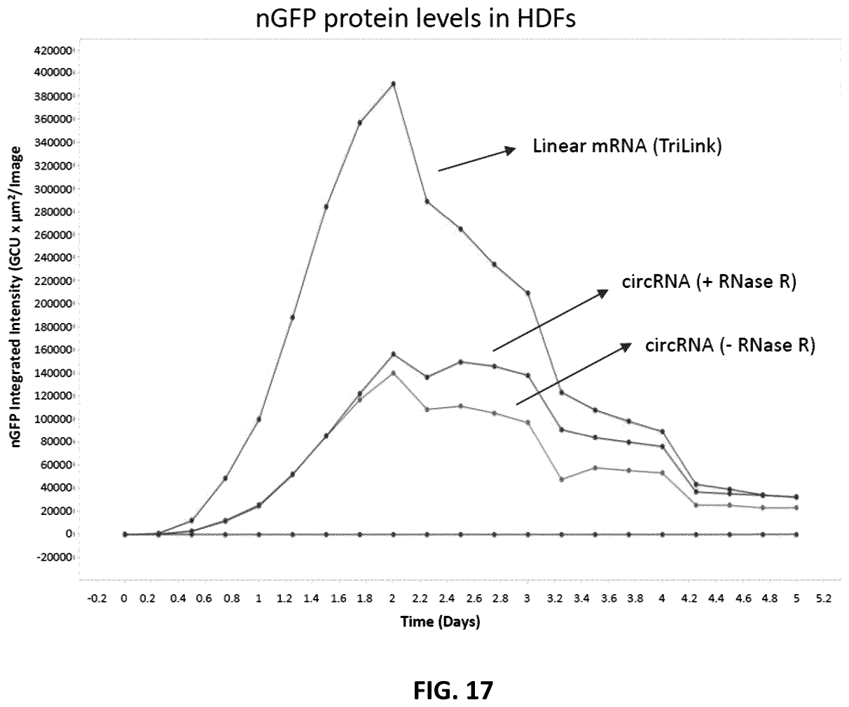

FIG. 17 provides the results of nGFP protein expression in fibroblasts (HDFs) after RNA transfection with RNAiMAX (50 ng/24-well) as analyzed by IncuCyte. A single transfection of nGFP coding linear mRNA, nGFP coding circRNA with RNase R treatment, or nGFP coding circRNA without RNase R treatment, was performed using RNAiMAX. After transfection, nGFP protein levels were measured every 6 hours by IncuCyte. Linear mRNA transfection resulted in a rapid increase of nGFP protein that peaked around 48 hours, followed by fast reduction in expression over the next two days. circRNA with and without RNaseR treatment showed similar patterns of protein expression—a smaller increase of nGFP protein level compared to linear mRNA, also peaked around 48 hours and gradually decreased over the next two days. Treating circRNA with RNaseR as a purification step resulted in a purer circRNA prep (from ˜60% to ˜90% circRNA, data not shown), with minimal effects on protein expression in fibroblasts.

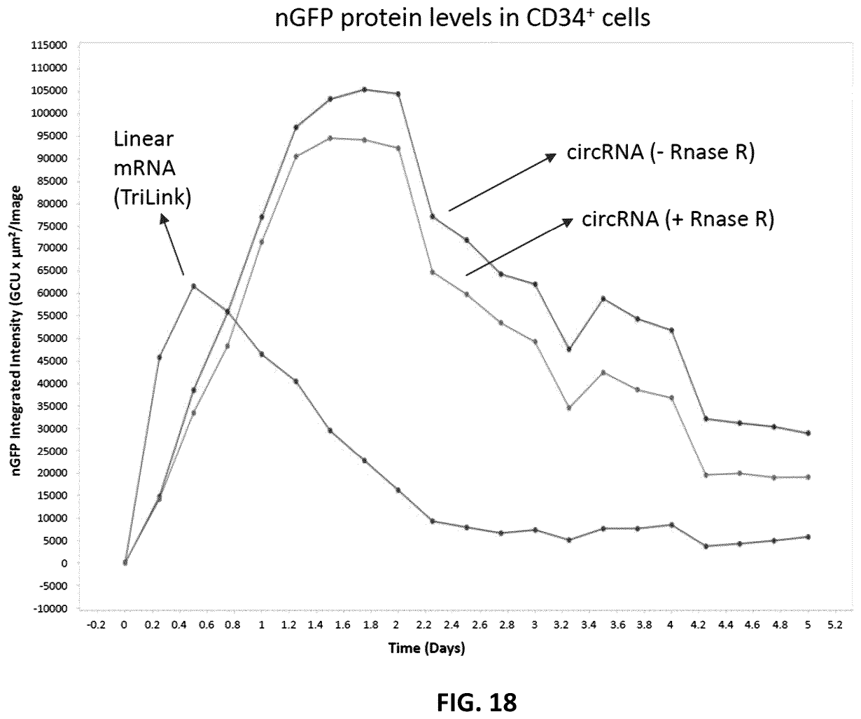

FIG. 18 provides the results of nGFP protein expression in CD34 cells after RNA transfection using Neon® (250 ng/6 well) as analyzed by IncuCyte. nGFP coding linear mRNA, nGFP coding circRNA with RNase R treatment, or nGFP coding circRNA without RNase R treatment, was transfected into CD34+ cells using Neon® electroporator. After one transfection, nGFP protein levels were measured every 6 hours by IncuCyte. In contrast to the observations in the fibroblasts, circRNA (both with and without RNaseR treatment) gave rise to higher and longer-lasting protein expression compared to linear mRNA. There was a minimal difference in the protein levels between RNaseR+ and RNaseR-circRNA.

### Example 6: Reduced Number of Reprogramming Factors to Reprogram CD34+ Cells

Experiments were performed to assess reprogramming of CD34+ cells in the absence of c-Myc, Lin28A, and/or Nanog. Also assessed was whether a single transfection of the C6 cocktail (OCT4, SOX2, KLF4, c-MYC, NANOG and LIN28A) was sufficient for reprogramming of CD34+ cells. Reprogramming results with circRNA reprogramming factors were compared to linear mRNA cocktails.

Conditions tested in these experiments are outlined in Table 12.

TABLE 12
Summary of Reprogramming Conditions*

| | | | | RNA (pg)/ |
|---|---|---|---|---|
| | Condition | Frequency of Tx | Number of Tx | cell/Tx |
| 1 | C6 | Alternate | 2 (d 0, d 2) | 3.2 |
| 2 | C6 | Once | 1 (d 0) | 3.2 |
| 3 | C6 | Alternate | 2 (d 0, d 2) | 8 |
| 4 | C6 | Once | 1 (d 0) | 8 |
| 5 | C5-OSKNL | Alternate | 2 (d 0, d 2) | 7 |
| 6 | C4-OSKM | Alternate | 2 (d 0, d 2) | 6.3 |
| 7 | C3-OSK | Alternate | 2 (d 0, d 2) | 5.2 |
| 8 | S6 (Stemgent) | Alternate | 2 (d 0, d 2) | 4.3 |
| 9 | L6 (TriLink) | Alternate | 2 (d 0, d 2) | 5.4 |
| 10 | Mock | Alternate | 2 (d 0, d 2) | — |
| 11 | CD34 | NA | NA | — |

*The amount RNA (pg) per cell per Tx is adjusted according to the number of factors being transfected, as well as the molecular weight of RNA for each factor.

Reprogramming cocktails tested were as follows
(i) C6 cocktail—circRNA, separately encoding OCT4, SOX2, KLF4, cMYC, NANOG and LIN28A (OSKMNL). Stoichiometry of the 6 reprogramming factors OSKMNL is 3:1:1:1:1:1
(b) C5 (cMyc dropout)—circRNA, separately encoding OCT4, SOX2, KLF4, NANOG and LIN28A (OSKNL). Stoichiometry of the 5 reprogramming factors OSKNL is 3:1:1:1:1:1
(i) C4 (Nanog and Lin28A dropout)—circRNA, separately encoding OCT4, SOX2, KLF4, and cMYC (OSKM). Stoichiometry of the 4 reprogramming factors OSKM is 3:1:1:1
(ii) C3 (cMyc, Nanog and Lin28A dropout)—circRNA, separately encoding OCT4, SOX2 and KLF4 (OSK). Stoichiometry of the 3 reprogramming factors is 3:1:1.

(c) Linear mRNA—Cocktails from Stemgent reprogramming kit and linear mRNA purchased from Trilink were tested—each encoded 6 factors OCT4, SOX2, KLF4, cMYC, NANOG and LIN28A (OSKMNL)

Note, the 6 factors used for reprogramming in the Stemgent(S), TriLink (L), and circRNA (C) cocktails are identical. However, the total molecular weights of constructs that encode these factors are different. Therefore, this impacted the total RNA delivered to each cell per transfection (as shown in Table 12).

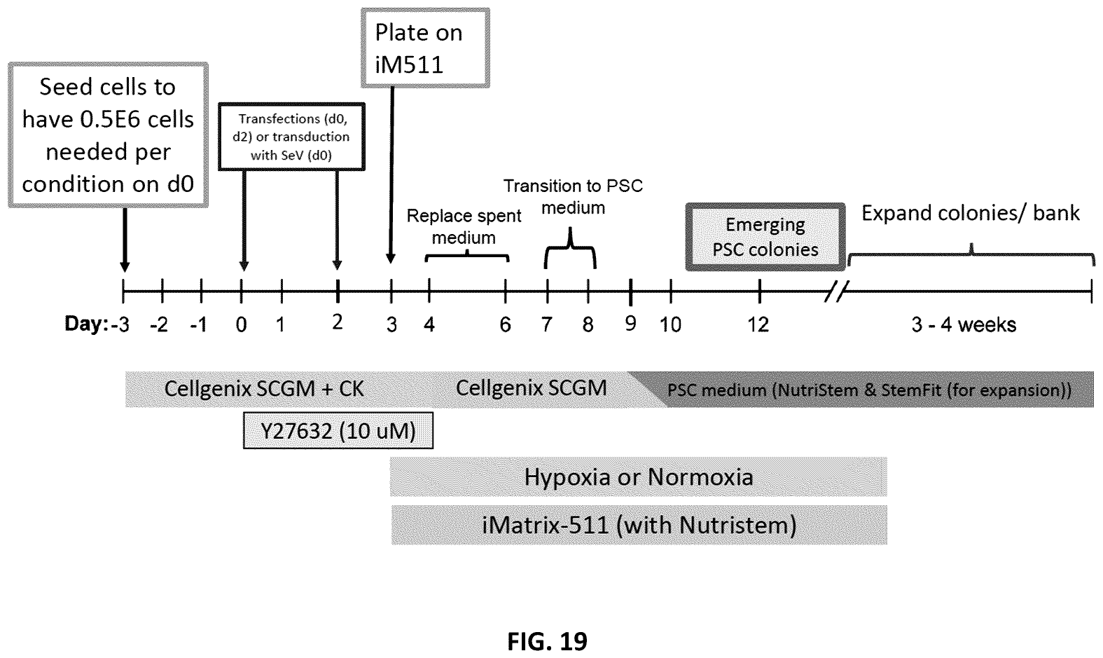

A summary of the experimental protocol is outlined in FIG. 19.

Briefly, cord blood-derived CD34+ cells were expanded for 3 days (d-3 to do) in SCGM media (Cellgenix) containing 5 cytokines—SCF, FLT3L, TPO, IL3 and IL6 (each at 100 ng/mL). On d0, 0.5×10⁶ cells per condition were pelleted, washed, and resuspended in 100-120 μL of buffer for transfection by electroporation. Cells were electroporated with a Neon electroporator set at 1600V, 10 ms pulse width, 3 pulses.

As negative controls, mock transfections (with Rnase, Dnase-free water) were done simultaneously as other transfections (Condition 10 in Table 12). CD34+ cells that were not transfected was also included as a negative control (Condition 11 in Table 12). Both controls had the same number of cells as all test conditions (0.5×10⁶ cells/condition). The positive control used in this experiment was an SeV Cytotune 2.0 encoded reprogramming cocktail encoding Oct4, Sox2, Klf4, and c-Myc.

Cells were resuspended in complete SCGM media containing 10 PM Y27632 after every transfection and seeded on 24-well non-adherent plates. On the day after the last transfection (d3), cell count and viability were determined for each condition including the controls (mock and CD34-only conditions). All cells were plated in iMatrix-511 coated 12-well tissue culture-treated plate with complete SCGM media (with cytokines) and 10 UM Y27632. Cells were grown in 5% O2 (hypoxia incubator).

On days 4 to 6, half of the media (1 mL) from each well was replaced with 1 mL SCGM media without cytokines, with 10 μM Y27632 supplemented to the media only until d4. On day 7, half of the media (1 mL) from each well was replaced with 1 mL of PSC media (Nutristem hPSC-XF for cells grown on iM511 or Stemfit media for cells grown on Vtn). From day 8 onwards, until iPSC colonies were ready to be picked, all media in each 6 well was replaced with 2 mL PSC media.

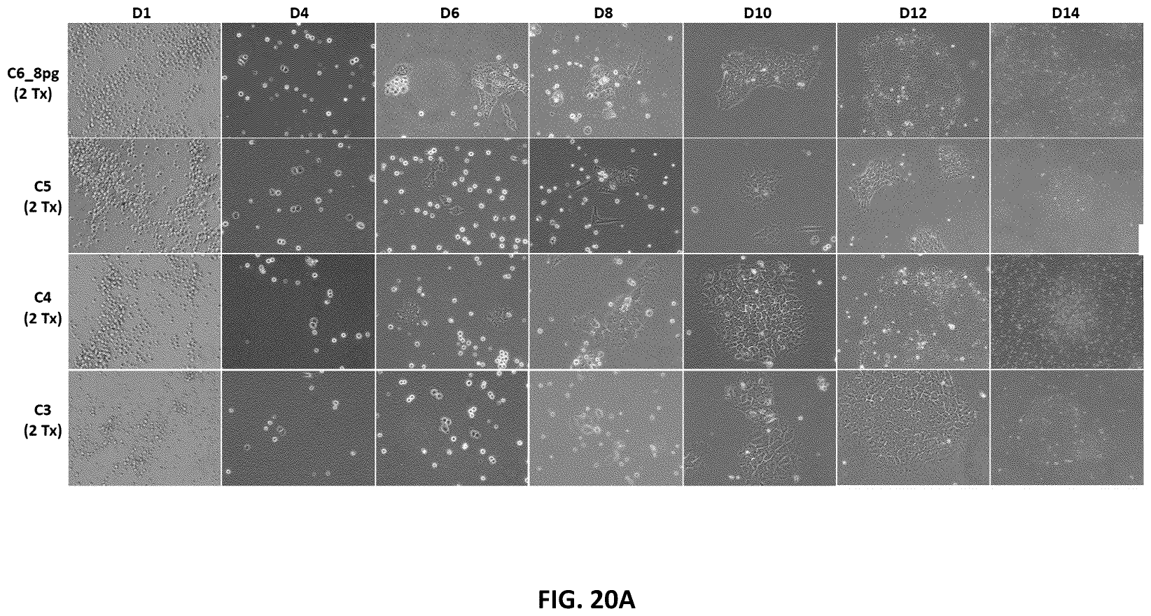

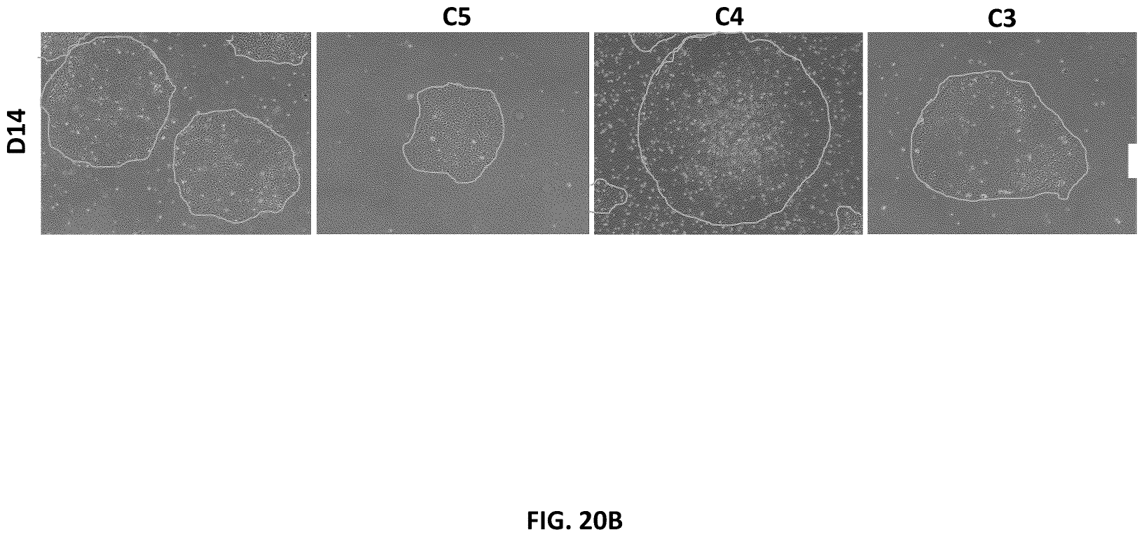

As shown in FIG. 20A, the C6 cocktail successfully gave rise to colonies with typical iPSC morphology. The C4 cocktail, which still contained cMyc but excluded Nanog and Lin28A, also resulted in large numbers of iPSC colonies (similar to C6). Importantly, although reprogramming with C5 and C3 cocktails, both of which excluded c-Myc, demonstrated a lower reprogramming efficiency compared to the C6 and C4 cocktails, these conditions also gave rise to iPSC colonies. Accordingly, there were fewer colonies in the C5 and C3 cultures compared to the C6 and C4 cultures. Additional results are shown in FIG. 20B, wherein the C5, C4, and C3 cocktails all reprogram CD34+ cells, but illustrates the smaller colonies and lower overall efficiency observed in the absence of c-Myc.

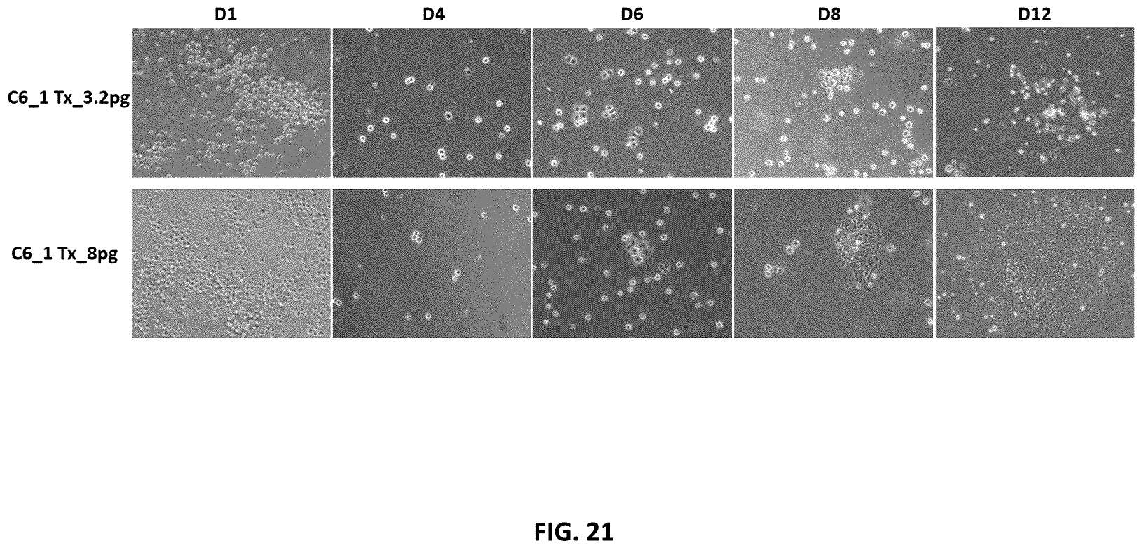

FIG. 21 shows the morphological progression of CD34+ reprogramming using only one transfection of the C6 circRNA cocktail. Two RNA amounts were tested-8 pg per cell and 3.2 pg per cell. Adherent cell clusters emerged in the cultures transfected with 8 pg RNA per cell around day 8 and continued to increase in size and became iPSC-like colonies. However, one transfection of 3.2 pg RNA per cell did not result in successful reprogramming of CD34+ cells. This demonstrates that CD34+ cells can be reprogrammed by a single delivery of circRNA encoding reprogramming factors provided sufficient concentrations of RNA are used.

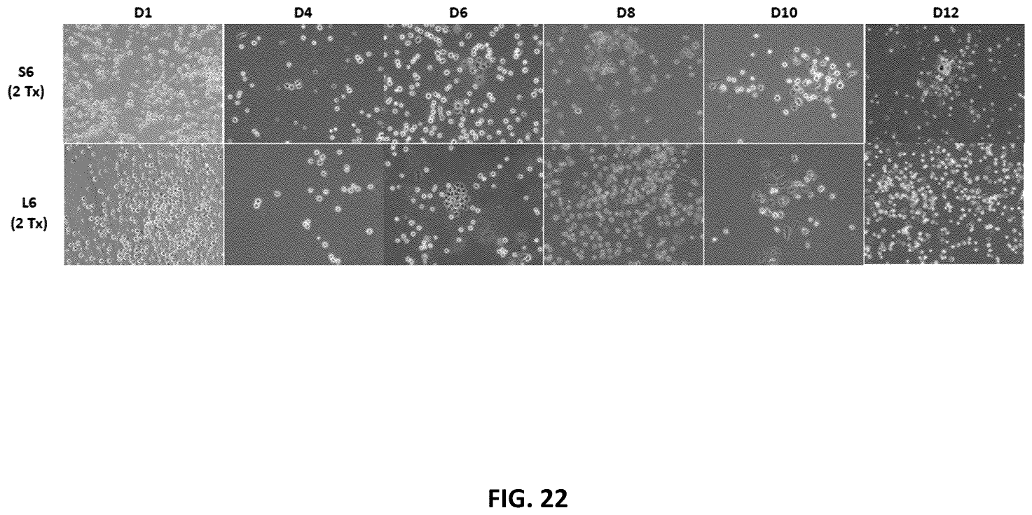

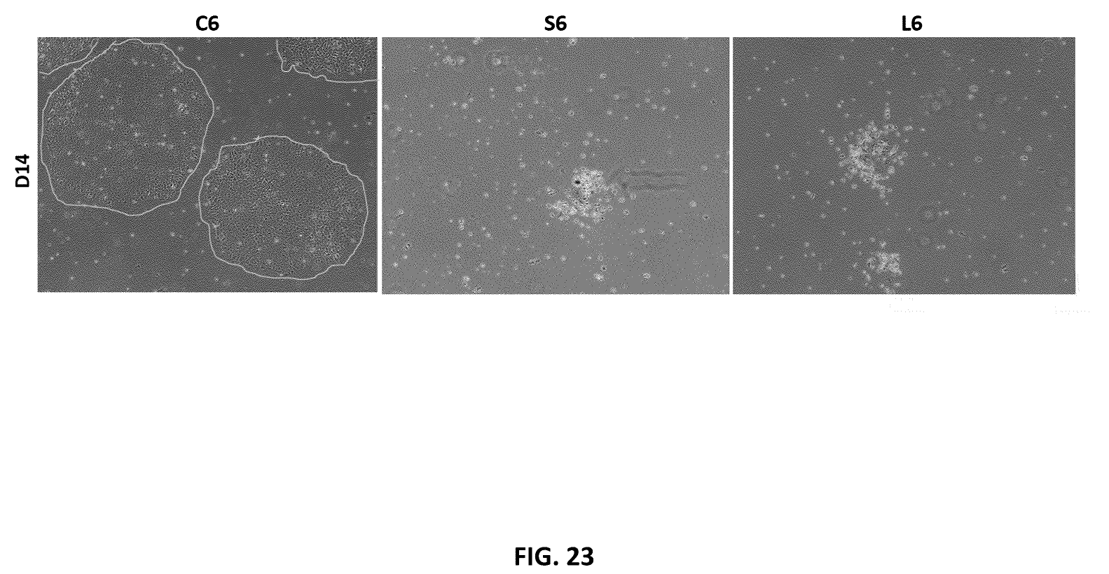

FIG. 22 shows the morphological progression of CD34+ reprogramming using linear mRNA. Reprogramming RNA cocktails from ReproCell/Stemgent's StemRNA 3^{rd} Gen Reprogramming kit (S6, OSKMLN) and a linear mRNA cocktail comprising individual linear mRNA purchased from Trilink (L6, OSKMLN) were used to reprogram CD34+ cells. Two transfections on day 0 and 2 were conducted. However, no iPSC colonies were formed in either S6 or L6 cultures. A comparison between the morphological progression observed with the C6 cocktail the progression observed with the S6 and L6 cocktail is provided in FIG. 23.

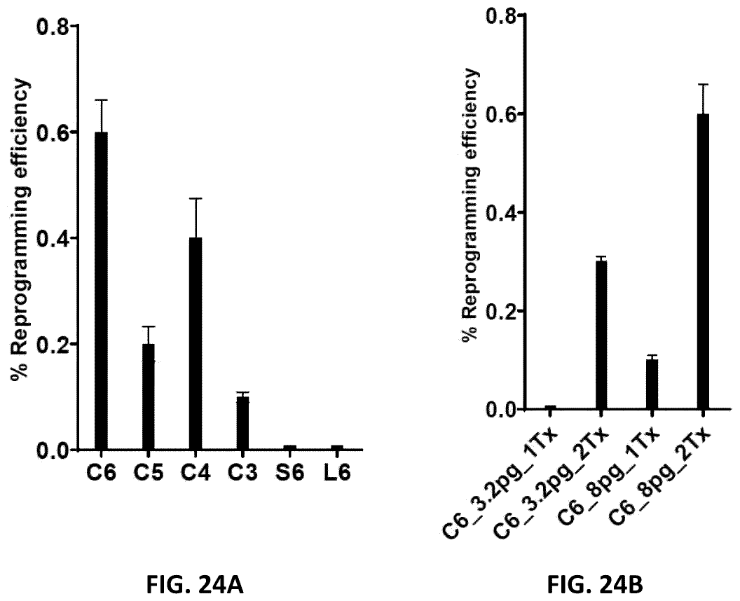

FIG. 24 demonstrates the reprogramming efficiency of CD34+ cells measured using IncuCyte. The reprogramming efficiency for circRNA cocktails C6, C5, C4 and C3, as well as linear mRNA cocktails from Stemgent (S6) and Trilink (L6) are compared in FIG. 24A. The reprogramming CD34+ cells using circRNAC6 cocktail with one or two transfections is shown in FIG. 24B. Two different RNA amounts per cell per transfection (3.2 pg and 8 pg) were tested. Note that only higher RNA amount (8 pg) resulted in reprogrammed iPSCs after one transfection with the C6 cocktail. Both high (8 pg) and low (3.2 pg) RNA amounts were able to reprogram CD34+ cells with two transfections.

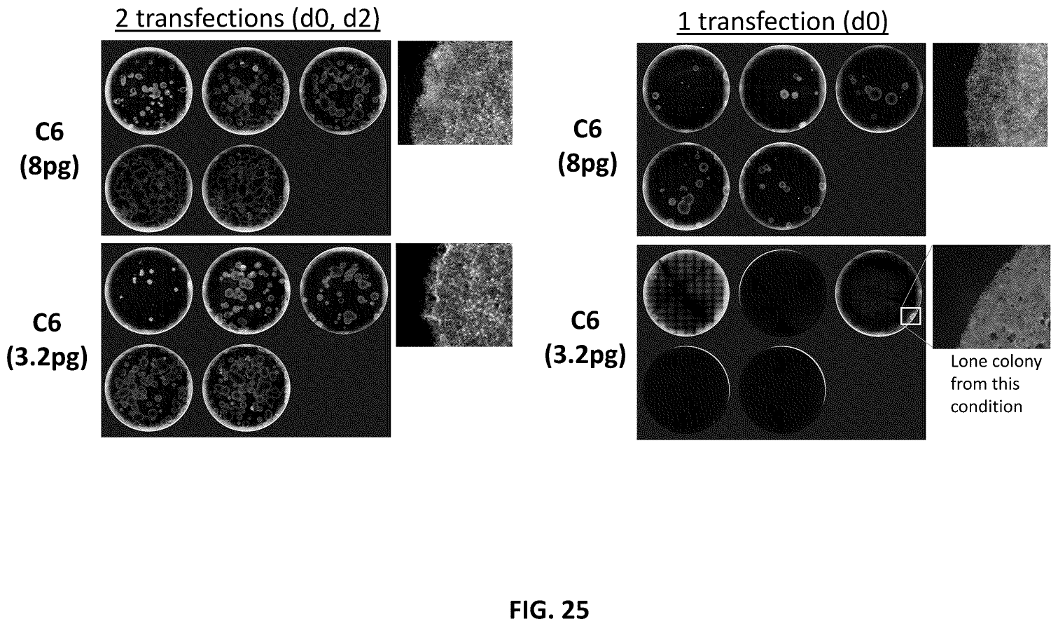

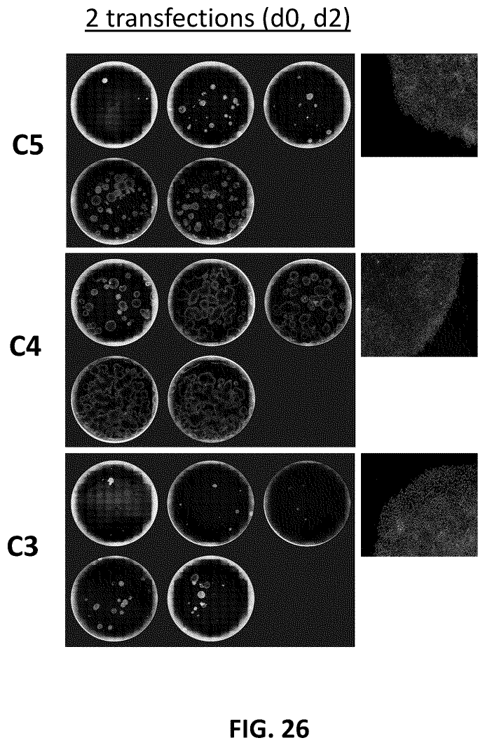

FIG. 25 and FIG. 26 show staining with pluripotency markers Tra-1-81 (green) and OCT4 (red) for 6 well plates transfected with various concentrations of the C6 cocktail (FIG. 25) and the C3, C4, C5 cocktails (FIG. 26). Whole-well images were scanned at 4× using IncuCyte. Different wells represent different seeding densities (from left to right, top to bottom: 12.5 k, 25 k, 25 k, 50 k, and 50 k). Tra-1-81− and Oct4-double positive areas are presumed iPSC colonies.

These experiments demonstrate the following conclusions:

1. The use of circRNA enables reprogramming with minimal numbers of factors. While excluding c-Myc (C5 and C3 cocktails) did reduce the rate of morphological progression to iPSCs as well as the overall reprogramming efficiency compared to the rate of progression and efficiency observed with the C6 cocktail (OSKMLN), these data demonstrate successful reprogramming of CD34+ cells in the absence of c-Myc. Similarly, exclusion of Nanog, Lin28 and c-Myc (C3 cocktail) resulted in reduction in reprogramming efficiency, although this reduction was not significant compared to the C5 cocktail. In other words, C5 and C3 were relatively similar in their reprogramming efficiencies. Even with the reduced efficiency, colonies with typical iPSC morphology and expressing pluripotency markers could be found in both C5 and C3 cultures.

2. Excluding Nanog and Lin28 while retaining c-Myc (C4 cocktail) showed reprogramming progression and efficiency similar to the C6 cocktail. This suggests that when circRNA is used to deliver reprogramming factors, Nanog and Lin28 are not essential for reprogramming of CD34+ cells to iPSCs.

3. c-Myc promotes efficient circRNA reprogramming in CD34+ cells. However, when c-Myc is absent circRNA reprogramming can still occur (see FIGS. 20A and 20B), albeit at a lower efficiency (lower rate of iPSC colony formation and lower numbers of iPSC colonies) compared to cocktails containing c-Myc.

4. CD34+ cells can be reprogrammed with a single transfection of the C6 cocktail. These experiments demonstrate the importance of RNA concentration in order to achieve successful reprogramming. At 8 pg RNA per cell, reprogramming was successful, while at 3.2-pg RNA per cell, no iPSC colonies were formed.

5. Linear mRNA cocktails (S6 and L6) analogous (in molar amounts and stoichiometry) to C6 circRNA cocktail failed to reprogram CD34+ cells, following the same transfection/electroporation protocol (two transfections on day 0 and day 2) used for analogous circRNA reprogramming cocktails. These experiments demonstrate that circular RNA reprogramming is uniquely suitable for footprint free suspension culture reprogramming, and these circular RNA constructs provide beneficial functional effects compared to analogous linear mRNA constructs.

### Example 7: Characterization of circRNA-Reprogrammed iPSCs in Long-Term Culture

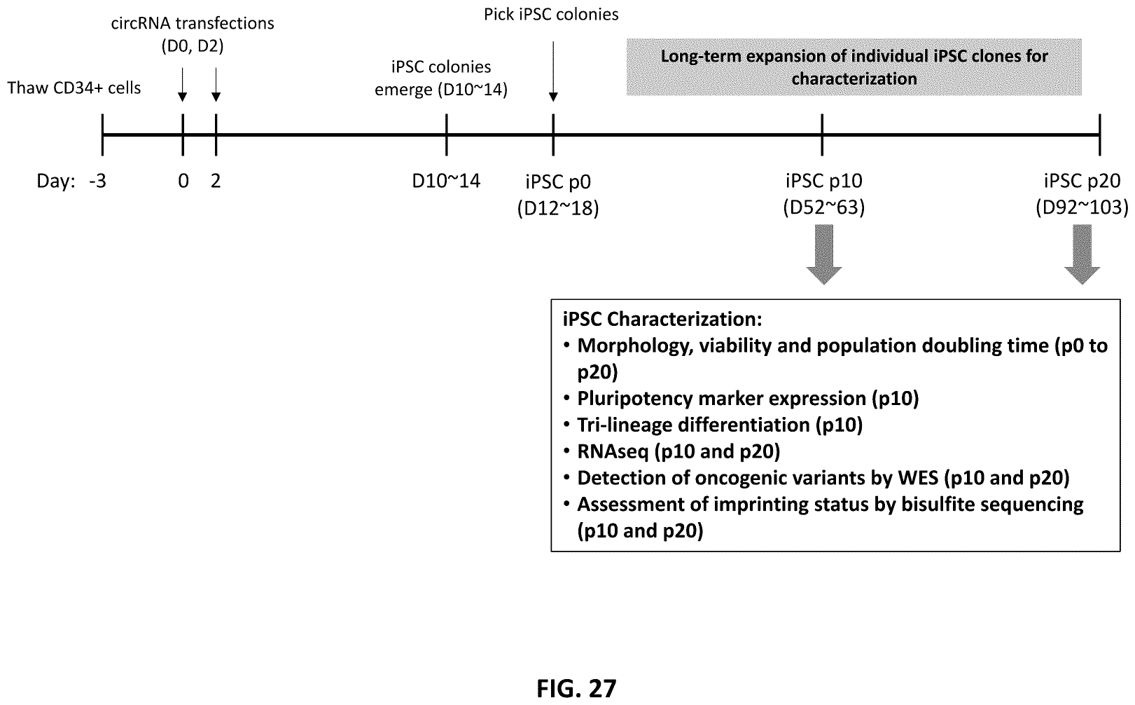

Experiments were performed to characterize iPSCs generated according to the methods described in the preceding example in long-term culturing conditions up to 20 cell passages. A summary of the experimental protocol is outlined in FIG. 27. Briefly, after two electroporations (DO and D2) of circRNA reprogramming cocktails, iPSC colonies typically emerged between day 10 to 14 and were ready to be picked by day 18. Individual iPSC colonies were manually picked and cultured in StemFit Basic 03 media up to passage 20 for characterization. Versene solution (Thermo Fisher) was used for detaching and dissociating iPSCs into small clumps during passaging. ROCK inhibitor Y-27632 was added on passaging days and removed the following day. At each passaging, iPSCs were seeded at a specific density which allowed for a consistent passaging cycle of 4 days per passage for all clones. Morphology, viability, population doubling time, and ability to differentiate into the three different germ layers were evaluated. Reprogramming conditions used in these experiments for each iPSC clone are defined by Table 13.

TABLE 13
Reprogramming protocols

| Protocol | | RNA(pg)/ | |
|---|---|---|---|
| Ref | Reprogramming Cocktail | cell/Tx | # Tx |
| C14_2.5X | circRNA encoding OSKMNL + linear | 13 pg | 2 |
| | RNA encoding E3/K3/B18R + 5 miRs | | |
| | (miR302a, -b, -c, -d and miR367) | | |
| C6_5X | circRNA encoding OSKMNL (3:1:1:1:1:1) | 15 pg | 2 |
| C6_2.5X | circRNA encoding OSKMNL (3:1:1:1:1:1) | 8 pg | 2 |
| C4_20X | circRNA encoding OSKM (3:1:1:1) | 45.6 pg | 2 |
| C4_10X | circRNA encoding OSKM (3:1:1:1) | 22.8 pg | 2 |
| C4_7.5X | circRNA encoding OSKM (3:1:1:1) | 17.1 pg | |
| C4_5x | circRNA encoding OSKM (3:1:1:1) | 11.4 pg | 2 |
| C4_2.5X | circRNA encoding OSKM (3:1:1:1) | 5.7 pg | 2 |
| C3_10X | circRNA encoding OSK (3:1:1) | 19 pg | 2 |
| C3_5X | circRNA encoding OSK (3:1:1) | 9.5 pg | 2 |
| C3_2.5X | circRNA encoding OSK (3:1:1) | 4.8 pg | 2 |

Overall reprogramming efficiencies for various protocols used are provided in Table 14. As shown, CD34+ cells can be successfully reprogrammed with the C6, C4, and C3 cocktail, with the C6 and C4 yielding the higher reprogramming efficiency.

TABLE 14
Summary of Reprogramming Success Rate and Efficiency

| | | No. exp | Repro- |
|---|---|---|---|
| | | producing | gramming * |
| circRNA (pg/cell/Tx) | | iPSC colonies | efficiency |
| C6 | 15 pg (5X_2Tx) | 4 of 4 | +++ |
| (OSKMLN) | 8 pg (2.5X_2Tx) | 4 of 4 | ++ |
| C4 | 45.6 pg (20X_2Tx) | 1 of 1 | + |
| (OSKM) | 22.8 pg (10X_2Tx) | 3 of 4 | ++ |
| | 17.1 pg (7.5X_2Tx) | 2 of 2 | ++ |
| | 11.4 pg (5X_2Tx) | 6 of 6 | +++ |
| | 5.7 pg (2.5X_2Tx) | 5 of 5 | ++ |
| C3 | 19 pg (10X_2Tx) | 0 of 2 | − |
| (OSK) | 9.5 pg (5X_2Tx) | 3 of 3 | + |
| | 4.8 pg (2.5X_2Tx) | 3 of 4 | + |

* +++ ≥ 0.1%; ++ ≥ 0.03%; + ≥ 0.003%

Cells at passage 0 were grown for 4-7 days prior to passage. Cells from subsequent passages were passaged every 4 days. Between days 52 to 63 post electroporation, the iPSC cell cultures at their tenth passage were harvested, stained, and analyzed by flow cytometry for OCT4, SSEA4, and TRA 1-81 expression. The positive control used in this experiment was commercially available iPSCs derived from episomal vectors encoding Oct4, Sox2, Klf4, Myc, Nanog, Lin28 and SV40 (epilPSC, A18945, Thermo Fisher). 293 T cells were used as a negative staining control. As shown in Table 15, reprogramming with the C14, C6, C4 and C3 cocktails led to cultures with a high percentage of iPSCs expressing OCT4, SSEA4, and TRA 1-81 (markers of pluripotency) when characterized at passage 10.

TABLE 15
Expression of Pluripotency Markers after Ten Cell Passages

| | | | | | % TRA |
|---|---|---|---|---|---|
| | iPSC clone | Protocol | % OCT4 | % SSEA4 | 1-81 |
| Mixed | 206-4 | C14_2.5X | 94.62 | 98.83 | 97.01 |
| donor | 206-5 | C14_2.5X | 94.83 | 99.06 | 97.71 |
| clones | 206-6 | C14_2.5X | 94 | 98.95 | 97.06 |
| (p10) | 281-2 | C6_2.5X | 98.29 | 99.37 | 99.41 |
| | 281-13 | C6_2.5X | 96.9 | 98.94 | 98.79 |
| | 281-14 | C6_2.5X | 96.11 | 98.72 | 98.27 |
| | 349-5 | C4_2.5X | 93.58 | 95.26 | 97.79 |
| | 349-6 | C4_2.5X | 94.91 | 95.89 | 97.88 |
| | 349-7 | C4_2.5X | 95.66 | 98.49 | 98.15 |
| | | epilPSC | 96.9 | 99.31 | 99.72 |
| Single | 245-5 | C4_5X | 89.78 | 95.15 | 89.56 |
| Donor | 245-6 | C4_5X | 91.24 | 96.96 | 90.94 |
| Clones | 245-7 | C4_5X | 88.86 | 95.14 | 90.94 |
| (p10) | 245-10 | C4_2.5X | 83.24 | 95.96 | 92.88 |
| | 245-11 | C4_2.5X | 83.99 | 95.41 | 90.99 |
| | 245-12 | C4_2.5X | 78.77 | 97.36 | 86.97 |
| | 245-1 | C6_5X | 81.36 | 96.89 | 90.62 |
| | 245-2 | C6_5X | 84.27 | 96.63 | 90.88 |
| | 245-3 | C6_5X | 75.44 | 96.52 | 81.99 |
| | 245-13 | C3_5X | 79.09 | 96.29 | 88.31 |
| | 245-16 | C3_5X | 90.84 | 99.53 | N/A |
| | | 293 T | 0.56 | 0.53 | 0.01 |
| | | epilPSC | 91.49 | 98.71 | 98.69 |

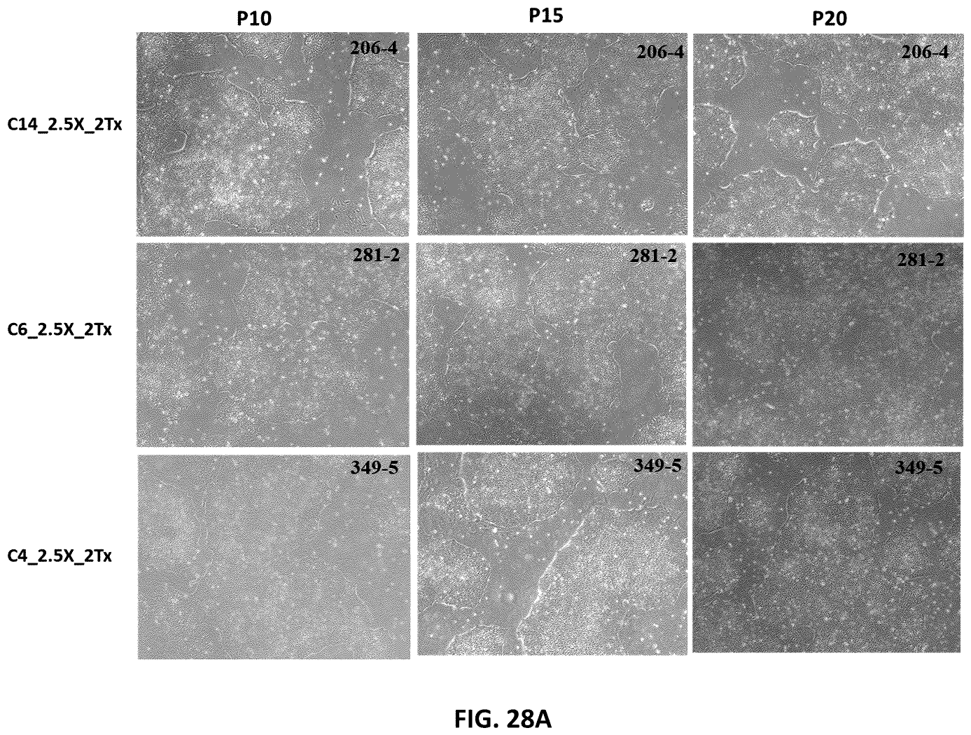

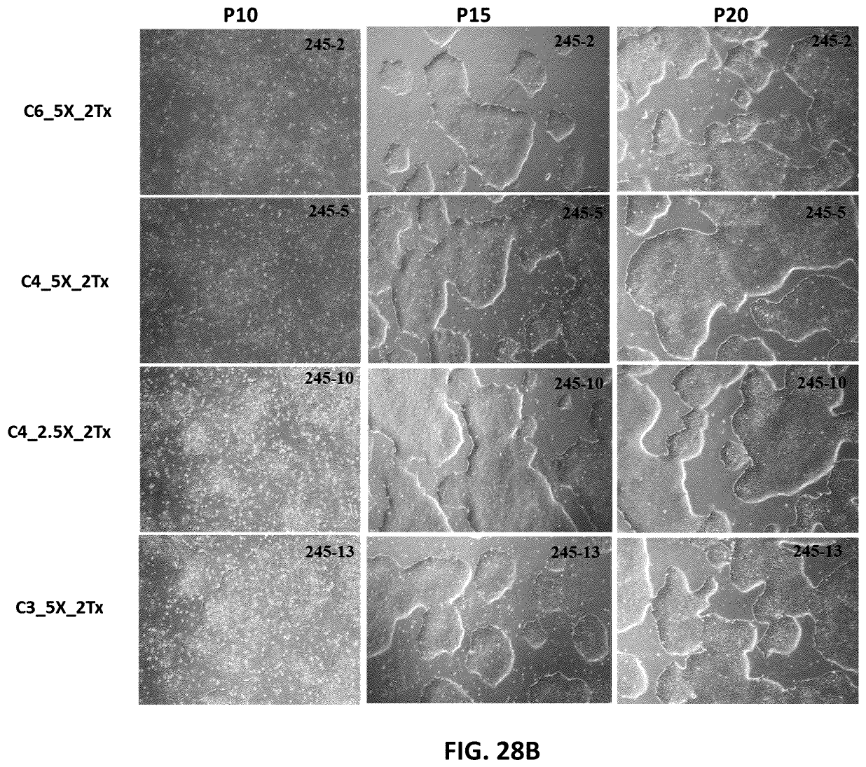

As shown in FIG. 28A, the iPSC clones derived from mixed donor CD34+ cells with reprogramming cocktails C14, C6, and C4 gave rise to colonies with typical iPSC morphology, and this was maintained through passage 20. Similarly, FIG. 28B shows the iPSC clones derived from single donor CD34+ cells with reprogramming cocktails C6, C4, and C3 also gave rise to colonies with typical iPSC morphology, and this was maintained through passage 20.

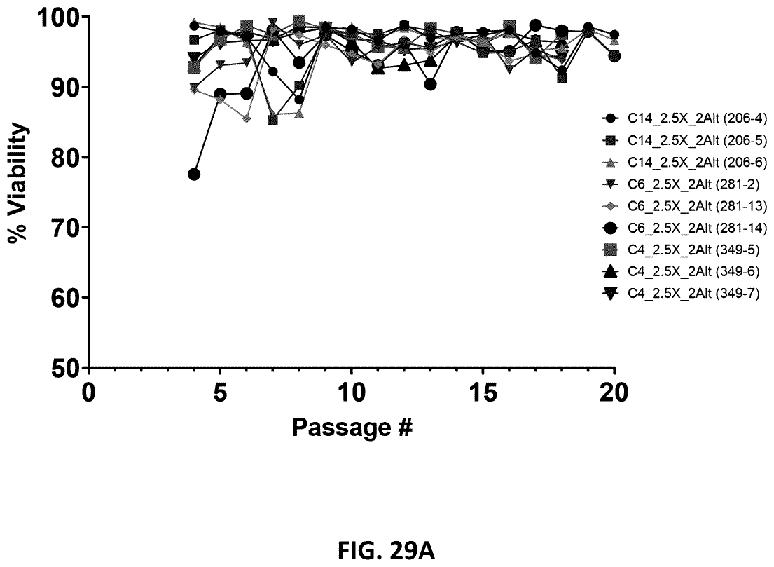

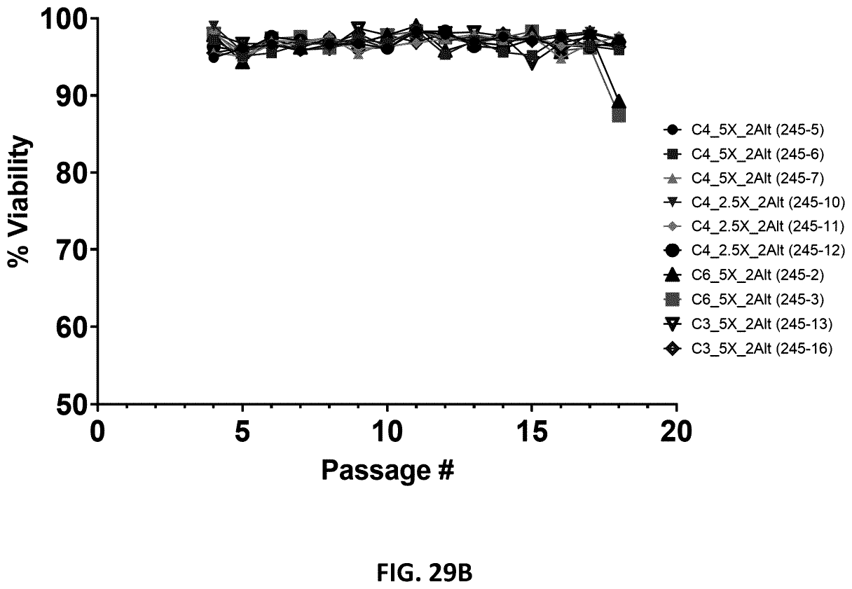

Furthermore, as shown in FIG. 29A and FIG. 29B, iPSC clones exhibited high viability in long-term culture (i.e., through 20 cell passages), as analyzed by nucleocounter NC_200. “Alt” as used in sample labeling denotes “alternate days,” or that the clone was generated by two circRNA electroporations on day 0 and day 2, as described above. iPSC clones derived from mixed donor (FIG. 29A) cord blood CD34+ cells maintained 75-98% viable at each cell passage from passage 4 to passage 20, with most clones remaining over 90% viable. With the exception of two clones being around 85% viable at passage 18, all clones derived from single donor (FIG. 29B) cord blood CD34+ cells were consistently over 90% viable at each cell passage from passage 4 to passage 18.

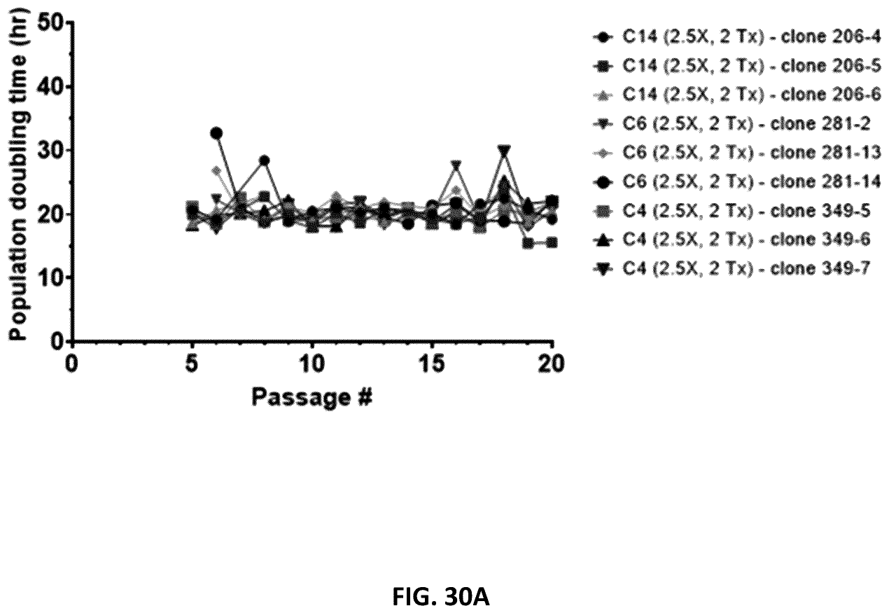

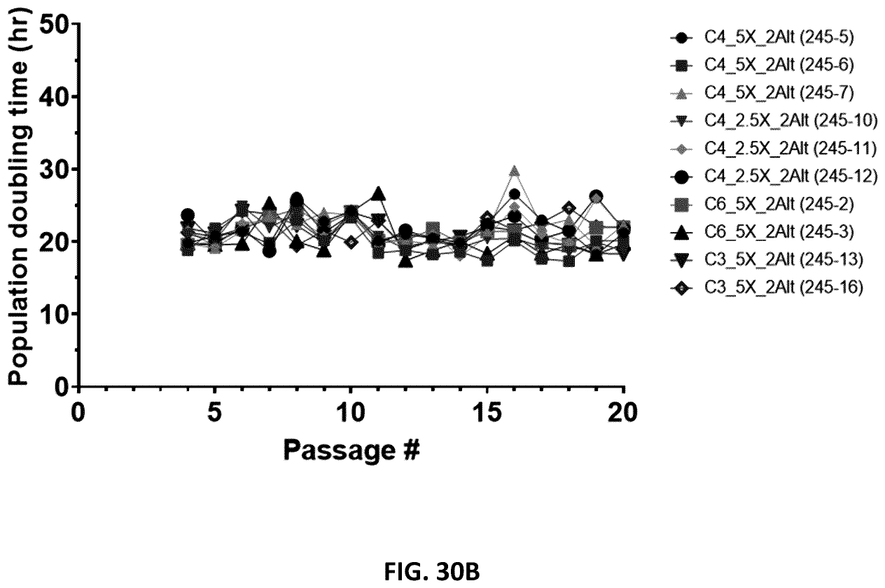

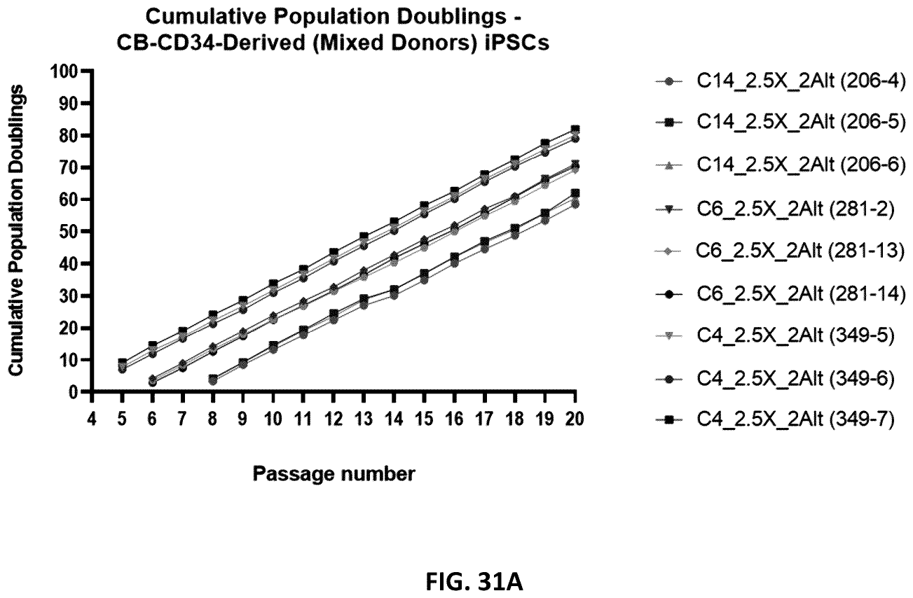

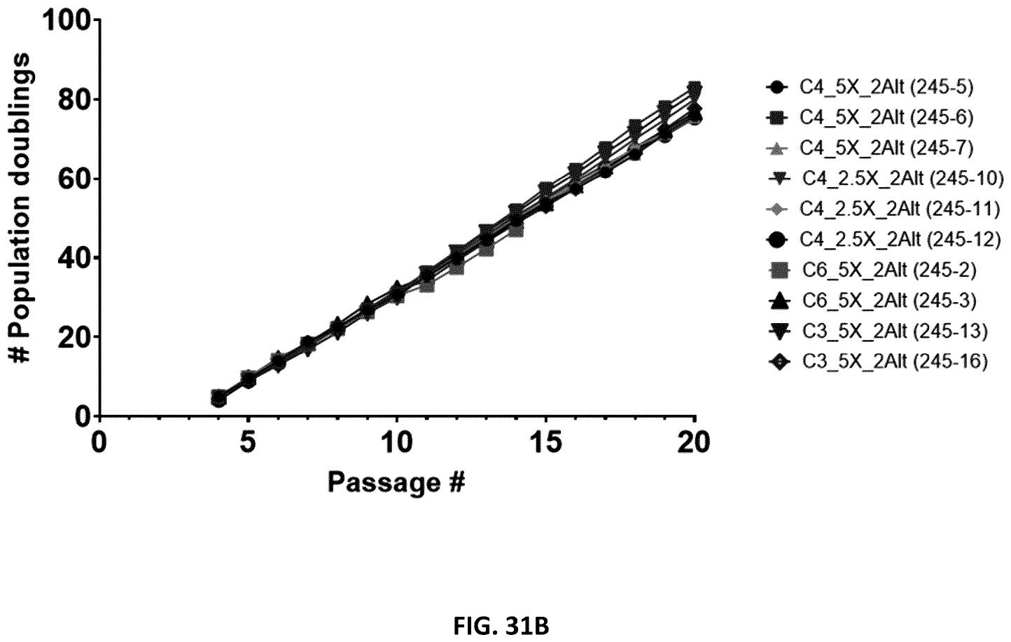

Additionally, as shown in FIG. 30A and FIG. 30B, circRNA-derived iPSCs exhibited consistent population doubling time, as calculated based on viable cell numbers on each passaging day, over extended culture. iPSC clones derived from mixed donor (FIG. 30A) and single donor (FIG. 30B) cord blood CD34+ cells doubled in number between 18-24 hours on average, consistent with typical iPSC doubling time, from passage 4 and up to passage 20. The passage numbers from which population doubling time started being recorded were variable between clones (from p5, p6 and p8), resulting in three groups of parallel lines in FIG. 31A. The cumulative population doublings of the circRNA-derived iPSCs from mixed donor CD34+ cells were consistently between 60-80 fold at passage 20, as shown in FIG. 31A, while the cumulative population doublings of the circRNA-derived iPSCs from single donor CD34+ cells were consistently between 70-80 fold at passage 20, as shown in FIG. 31B.

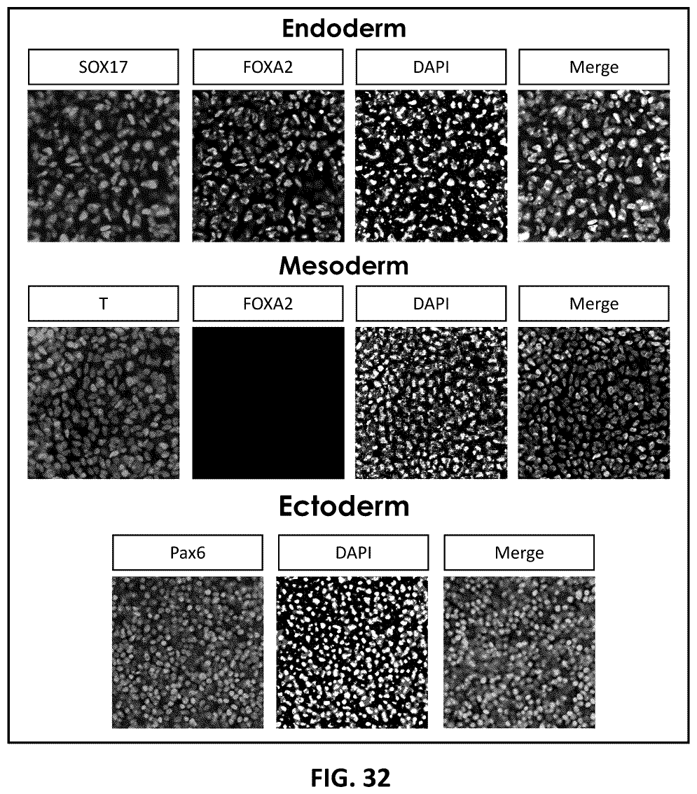

Moreover, FIG. 32 shows that after reprogramming with the C14, C6, C4 or C3 cocktail, the resulting iPSC cells were able to give rise to all three primary germ cell layers. The endoderm expressed SOX17 and FOXA2. The mesoderm expressed Brachyury (T) and not FOXA2. The ectoderm expressed PAX6. RNA sequencing (RNA seq) analysis was also performed on the resulting iPSC cultures. Three separate RNA seq runs were performed on the resulting iPSC cultures. The first sequencing run included samples from 11 VR1+VR2 clones and 9 clones from mixed donor CD34+ cells reprogrammed with C14, C6, and C4 cocktails. The second and third sequencing runs each included samples from 10 clones, run in duplicates, from single donor CD34+ cells reprogrammed with C6, C4 and C3 cocktails, with cord blood cells used as a control. CELL net analysis was used to classify samples into cell types based on their expression of gene regulatory signatures.

All circRNA-derived iPSC clones had a gene signature that matched the signature of embryonic stem cells (ESC), defined by CellNet analysis based on the gene regulatory networks (GRNs) of ESCs composed of 206 genes. Of the 206 genes associated with ESC GRN, expression of LIN28A, LIN28B, NANOG, POU5F1, and SOX2 were high across all circRNA-derived iPSC samples. Principal component analysis (PCA) and Pearson correlation confirmed there were no batch effects and showed no clustering between C6, C4 and C3 (data not shown).

Taken together, the data show that circRNA-reprogrammed iPSCs from different donors and reprogrammed using the C14, C6, C4 and C3 cocktails as described in the present disclosure are stable in their morphology, remain viable, continue to undergo cell division at a similar rate, and maintain consistent cell identity that closely resembles ESCs after multiple cell passages and under long term cell culturing conditions.

### Example 8: Genetic and Epigenetic Stability of circRNA-Reprogrammed iPSCs

Experiments were performed to assess the genetic and epigenetic stability of circRNA-reprogrammed iPSCs after multiple cell passages and long-term culturing. G-Banding Karyotyping was performed for a total of 32 circRNA-reprogrammed iPSC clones at passage 10, of which 29 out of 32 clones were of normal karyotype. These 29 clones were then karyotyped again passage 20. As summarized in Table 16, over 85% of the clones had normal karyotype at passages 10 and 20, which is higher than the generally expected rate of 80%.

TABLE 16
Summary of G-Banding Karyotyping Results for
Detection of Chromosomal Abnormalities

| | No. clones with normal | No. clones with normal |
|---|---|---|
| Donor | karyotype (p10) | karyotype (p20) |
| Mixed Donors | 9 out of 9 | 8 out of 9 |
| | (including 1 with | (including 1 with |
| | non-clonal finding) | non-clonal finding) |
| Single Donor 1 | 10 out of 11 | 10 out of 10 |
| | (including 2 with | |
| | non-clonal finding) | |
| Single Donor 2 | 4 out of 6 | 3 out of 4 |
| Single Donor 3 | 6 out of 6 | 6 out of 6 |
| Total | 29 out of 32 | 27 out or 29 |

Whole genome sequencing was also performed on the same clones for detection of oncogenic variants. As summarized in Table 17, none of the clones presented with oncogenic TP53 variants. Furthermore, bisulfite genomic sequencing for detection of DNA methylation was performed on the same clones, and all clones showed DNA methylation at the IGF2/H19 imprinting control region consistent with parental DNA methylation, as shown in Table 17.

TABLE 17
Summary of Sequencing Results for Determining Genetic and
Epigenetic Stability of circRNA-derived iPSC clones

| | No. clones | No. clones | No. clones | No. clones |
|---|---|---|---|---|
| | with | with | consistent with | consistent |
| | oncogenic | oncogenic | parental | with parental |
| | TP53 | TP53 | imprinting at | imprinting at |
| | Variants | Variants | IGF2/H19 | IGF2/H19 |
| Donor | (p10) | (p20) | (p10) | (p20) |
| Mixed | 0 of 9 | N/A | 9 or 9 | N/A |
| Donors | | | | |
| Single | 0 of 11 | 0 of 10 | 11 of 11 | 10 of 10 |
| Donor 1 | | | | |
| Single | 0 of 3 | 3 of 3 | — | — |
| Donor 2 | | | | |
| Single | 0 of 6 | 0 of 6 | — | — |
| Donor 3 | | | | |

Taken together, these data demonstrate that circRNA-derived iPSCs are both genetically and epigenetically stable after long-term in vitro culturing.

### Example 9: Advantages of iPSC Reprogramming with circRNA

Based on experimental data presented herein, features of iPSC reprogramming with circRNA were compared to those of other reprogramming methods such as reprogramming with Sendai Virus, episomal vectors, and mRNA. Table 18 summarizes the advantages of iPSC reprogramming with circRNA. Reprogramming with circRNA was suitable for blood cells, did not risk genome integration, did not require clearance of exogenously introduced reprogramming factors in the iPSCs (once delivered, RNA is rapidly cleared within a few days due to short half-life), did not require the inclusion of p53 as a part of the reprogramming factor combination, did not require use of additional factors to mediate immune evasion, did not require use of microRNA, and allowed for myc-free reprogramming. Of the other reprogramming methods, use of Sendai Virus and episomal vectors required clearance of the reprogramming factors. Use of Sendai Virus, episomal vectors, and mRNA required the use of myc for efficient reprogramming. Furthermore, use of episomal vectors risked genome integration while use of mRNA was not suitable for reprogramming blood cells and required use of additional factors to mediate immune evasion and enhance reprogramming efficiency. Taken together, iPSC reprogramming with episomal vectors and mRNA is complex and prohibitive while reprogramming with Sendai Virus presents limited use in ocular and central nervous system indications. However, iPSC reprogramming with circRNA not only overcomes these limitations but also allows for reprogramming directly from blood cells in suspension, and therefore presents as a superior method.

TABLE 18
Summary of Features of Different Methods for iPSC Reprogramming
and Their Advantages and Disadvantages Compared to circRNA

| Reprogramming | Sendai | | | |
|---|---|---|---|---|
| system | Virus | Episomal | mRNA | circRNA |
| Blood cell | Yes | Yes | No | Yes |
| Reprogramming | | | | |
| Efficiency | ~0.1% | 0.01% | 1~3% (in | >2% (in |
| | | | fibroblasts) | fibroblasts) |
| | | | | 0.04~4% (in |
| | | | | CD34s) |
| Genome | No | Risk | No | No |
| Integration | | | | |
| Clearance | Yes | Yes | No | No |
| Required | | | | |
| p53 mutations | No | Yes | No | No |
| Immune Evader | No | No | Yes | No |
| Required | | | | |
| microRNA | No | No | Yes | No |
| Required | | | | |
| Myc-free | No | No | No | Yes |
| Reprogramming | | | | |

The foregoing is illustrative of the present invention and is not to be construed as limiting thereof. The invention is defined by the following claims, with equivalents of the claims to be included therein.

What is claimed is:

1. A method of producing an induced pluripotent stem cell (iPSC), the method comprising contacting a blood cell in suspension with a circular RNA encoding at least one reprogramming factor selected from Oct3/4, Klf4, Sox2, Nanog, Lin28, c-Myc, or L-Myc, or a fragment or variant thereof, and maintaining the cell under conditions under which the iPSC is obtained.

2. The method of claim 1, wherein the blood cell is selected from a T cell, a B cell, a natural killer (NK) cell, a natural killer T (NKT) cell, a peripheral blood mononuclear cell (PBMC), and a cord blood mononuclear cell (CBMC).

3. The method of claim 1, wherein the blood cell is selected from a T cell and an NK cell.

4. A method of producing an induced pluripotent stem cell (iPSC), the method comprising contacting a CD34+ cell in suspension with a circular RNA encoding at least one reprogramming factor selected from Oct3/4, Klf4, Sox2, Nanog, Lin28, c-Myc, or L-Myc, or a fragment or variant thereof, and maintaining the cell under conditions under which the iPSC is obtained.

5. The method of claim any one of claims 1-4, wherein the at least one reprogramming factor is a human or a humanized reprogramming factor.

6. The method of claim any one of claims 1-5, wherein the at least one reprogramming factor is Oct3/4, and wherein the Oct3/4 has the amino acid sequence of SEQ ID NO: 7, or a sequence at least 90% or at least 95% identical thereto.

7. The method of claim 3, wherein the circular RNA comprises a nucleic acid sequence of SEQ ID NO: 8, or a sequence at least 90% or at least 95% identical thereto.

8. The method of any one of claims 1-5, wherein the at least one reprogramming factor is Klf4, and wherein the Klf4 has the amino acid sequence of SEQ ID NO: 9 or 10, or a sequence at least 90% or at least 95% identical thereto.

9. The method of claim 8, wherein the circular RNA comprises a nucleic acid sequence of SEQ ID NO: 11, or a sequence at least 90% or at least 95% identical thereto.

10. The method of any one of claims 1-5, wherein the at least one reprogramming factor is Sox2, and wherein Sox2 has the amino acid sequence of SEQ ID NO: 12, or a sequence at least 90% or at least 95% identical thereto.

11. The method of claim 10, wherein the circular RNA comprises a nucleic acid sequence of SEQ ID NO: 13, or a sequence at least 90% or at least 95% identical thereto.

12. The method of any one of claims 1-5, wherein the at least one reprogramming factor is Nanog, and wherein the Nanog has the amino acid sequence of SEQ ID NO: 14 or 15, or a sequence at least 90% or at least 95% identical thereto.

13. The method of claim 12, wherein the circular RNA comprises a nucleic acid sequence of SEQ ID NO: 16, or a sequence at least 90% or at least 95% identical thereto.

14. The method of any one of claims 1-5, wherein the at least one reprogramming factor is Lin28A, and wherein the Lin28A has the amino acid sequence of SEQ ID NO: 17, or a sequence at least 90% or at least 95% identical thereto.

15. The method of claim 14, wherein the circular RNA comprises a nucleic acid sequence of SEQ ID NO: 18, or a sequence at least 90% or at least 95% identical thereto.

16. The method of any one of claims 1-5, wherein the at least one reprogramming factor is c-Myc, and wherein the c-Myc has the amino acid sequence of SEQ ID NO: 19 or 20, or a sequence at least 90% or at least 95% identical thereto.

17. The method of claim 16, wherein the circular RNA comprises a nucleic acid sequence of SEQ ID NO: 21, or a sequence at least 90% or at least 95% identical thereto.

18. The method of any one of claims 1-5, wherein the at least one reprogramming factor is L-Myc, and wherein the L-Myc has the amino acid sequence of any one of SEQ ID NO: 22-24, or a sequence at least 90% or at least 95% identical thereto.

19. The method of claim 18, wherein the circular RNA comprises a nucleic acid sequence of SEQ ID NO: 25, or a sequence at least 90% or at least 95% identical thereto.

20. The method of any one of claims 1-19, wherein the circular RNA is substantially non-immunogenic.

21. The method of claim 20, wherein the circular RNA comprises one or more M-6-methyladenosine (m⁶A) residues.

22. The method of any one of claim 1-21, wherein the circular RNA comprises from about 200 nucleotides to about 5,000 nucleotides.

23. The method of any one of claims 1-22, wherein the circular RNA comprises an internal ribosome entry site (IRES) operably linked to the protein-coding sequence.

24. The method of any one of claims 1-23, wherein the circular RNA encodes two or more reprogramming factors selected from Oct3/4, Klf4, Sox2, Nanog, Lin28, c-Myc, or L-Myc.

25. The method of any one of claims 1-5, wherein the method comprises contacting the cell with a first circular RNA encoding Oct4 or a fragment or variant thereof, a second circular RNA encoding Sox2 or a fragment or variant thereof, a third circular RNA encoding Klf4 or a fragment or variant thereof, a fourth circular RNA encoding C-Myc or a fragment or variant thereof, a fifth circular RNA encoding Lin28 or a fragment or variant thereof, and a sixth circular RNA encoding Nanog or a fragment or variant thereof.

26. The method of any one of claims 1-5, wherein the method comprises contacting the cells with one or more circular RNAs encoding one or more of a group of reprogramming factors consisting of Oct3/4, Klf4, Sox2, Nanog, Lin28, and c-Myc, or fragments or variants thereof.

27. A method of producing an induced pluripotent stem cell (iPSC), the method comprising contacting a blood cell or a CD34+ cell in suspension with six circular RNAs consisting of a first circular RNA encoding Oct4, a second circular RNA encoding Sox2, a third circular RNA encoding Klf4, a fourth circular RNA encoding C-Myc, a fifth circular RNA encoding Lin28, and a sixth circular RNA encoding Nanog, and maintaining the cell under conditions under which the iPSC is obtained.

28. A method of producing an induced pluripotent stem cell (iPSC), the method comprising contacting a blood cell or a CD34+ cell in suspension with three circular RNAs consisting of a first circular RNA encoding Oct4, a second circular RNA encoding Sox2, and a third circular RNA encoding Klf4, and maintaining the cell under conditions under which the iPSC is obtained.

29. The method of claim 28, wherein the method does not comprise contacting the blood cell or CD34+ cell with a circular RNA encoding any of Nanog, Lin28, and c-Myc.

30. A method of producing an induced pluripotent stem cell (iPSC), the method comprising contacting a blood cell or a CD34+ cell in suspension with three circular RNAs consisting of a first circular RNA encoding Oct4, a second circular RNA encoding Sox2, and a third circular RNA encoding Klf4, and a fourth circular RNA encoding C-Myc, and maintaining the cell under conditions under which the iPSC is obtained.

31. The method of claim 30, wherein the method does not comprise contacting the blood cell or CD34+ cell with a circular RNA encoding either of Nanog or Lin28.

32. A method of producing an induced pluripotent stem cell (iPSC), the method comprising contacting a blood cell or a CD34+ cell in suspension with three circular RNAs consisting of a first circular RNA encoding Oct4, a second circular RNA encoding Sox2, and a third circular RNA encoding Klf4, and a fourth circular RNA encoding Nanog, and a fifth circular RNA encoding Lin 28, and maintaining the cell under conditions under which the iPSC is obtained.

33. The method of claim 32, wherein the method does not comprise contacting the CD34+ cell with a circular RNA encoding c-Myc.

34. The method of claim any one of claims 1-33, wherein the cell is not contacted with any factor selected from E3, K3, B18R.

35. The method of claim any one of claims 1-33, wherein the cell is not contacted with any micro RNAs (miRs).

36. The method of claim any one of claims 1-33, wherein the cell is not contacted with one or more factor selected from E3, K3, B18R, one or more micro RNAs (miRs), or a combination thereof.

37. The method of any one of 35-36, wherein the miRs comprise miR302a, miR302b, miR302c, miR302d, and miR367.

38. The method of any one of claims 1-37, wherein the cell is directly contacted with the at least one circular RNA.

39. The method of any one of claims 1-38, wherein the cell is contacted with each of the at least one circular RNA once.

40. The method of any one of claims 1-38, wherein the method comprises contacting the cell with each of the at least one circular RNA two, three, four, or more times.

41. The method of any one of claims 1-38, comprising contacting the cell with each of the at least one circular RNA fewer than four times.

42. The method of any one of claims 1-38, comprising contacting the cell with each of the at least one circular RNA from 2 to 4 times.

43. The method of any one of claims 1-42, wherein the concentration of each of the at least one circular RNAs is at least 3 pg RNA/cell.

44. The method of any one of claims 1-42, wherein the concentration of each of the at least one circular RNAs is from about 5 pg RNA/cell to about 15 pg RNA/cell.

45. The method of any one of claims 1-44, wherein the contacting the cell is performed by electroporation.

46. The method of any one of claim 1-26 or 38-45, wherein the method comprises further contacting the cell with an RNA polynucleotide encoding one or more viral proteins selected from E3, K3, or B18R.

47. The method of claim 46, wherein:
(a) the B18R has a sequence of SEQ ID NO: 26, or a sequence at least 90% or at least 95% identical thereto;
(b) the E3 has a sequence of SEQ ID NO: 27, or a sequence at least 90% or at least 95% identical thereto; and/or
(c) the K3 has a sequence of SEQ ID NO: 28, or a sequence at least 90% or at least 95% identical thereto.

48. The method of any one of claim 1-26 or 38-47, wherein the method comprises further contacting the cell with one or more microRNAs (miRs).

49. The method of claim 48, wherein the miRs are selected from miR302a, miR302b, miR302c, miR302d, and miR367.

50. The method of any one of claims 1-49, wherein the method results in one or more of:
(i) an increase in the number of reprogrammed iPSC present at the end of culture compared to a method of producing an iPSC with one or more linear RNAs; and/or
(ii) a decrease in cell death at one or more timepoints during reprogramming compared to a method of producing an iPSC with one or more linear RNAs.

51. The method of any one of claims 1-49, wherein the method results in each of:
(i) an increase in the number of reprogrammed iPSC present at the end of culture compared to a method of producing an iPSC with one or more linear RNAs; and
(ii) a decrease in cell death at one or more timepoints during reprogramming compared to a method of producing an iPSC with one or more linear RNAs.

52. An iPSC produced using the method of any one of claims 1-51.

53. A differentiated cell derived from the iPSC of claim 52.

54. The differentiated cell of claim 53, wherein the differentiated cell is a muscle cell, a neural cell, a cell of the central nervous system, an ocular cell, a chondrocyte, an osteocyte, a tendon cell, a renal cell, a cardiomyocyte, a hepatocyte, an islet cell, a keratinocyte, a T-cell, or a NK-cell.

55. A method for reprogramming and editing the genome of a cell, the method comprising:
(i) contacting the cell with a recombinant circular RNA comprising a protein-coding sequence, wherein the protein-coding sequence encodes at least one reprogramming factor, and
(ii) contacting the cell with an enzyme capable of editing the DNA or RNA of the cell, or a nucleic acid encoding the same.

56. A method for reprogramming and editing the genome of a cell, the method comprising simultaneously contacting the cell with:
(i) a recombinant circular RNA comprising a protein-coding sequence, wherein the protein-coding sequence encodes at least one reprogramming factor, and
(ii) an enzyme capable of editing the DNA or RNA of the cell, or a nucleic acid encoding the same.

57. The method of claim 55 or 56, wherein the at least one reprogramming factor is a human or a humanized reprogramming factor.

58. The method of any one of claims 55-57, wherein the at least one reprogramming factor is Oct3/4, and wherein the Oct3/4 has the amino acid sequence of SEQ ID NO: 7, or a sequence at least 90% or at least 95% identical thereto.

59. The method of claim 59, wherein the recombinant circular RNA comprises a nucleic acid sequence of SEQ ID NO: 8, or a sequence at least 90% or at least 95% identical thereto.

60. The method of any one of claims 55-57, wherein the at least one reprogramming factor is Klf4, and wherein the Klf4 has the amino acid sequence of SEQ ID NO: 9 or 10, or a sequence at least 90% or at least 95% identical thereto.

61. The method of claim 60, wherein the recombinant circular RNA comprises a nucleic acid sequence of SEQ ID NO: 11, or a sequence at least 90% or at least 95% identical thereto.

62. The method of any one of claims 55-57, wherein the at least one reprogramming factor is Sox2, and wherein Sox2 has the amino acid sequence of SEQ ID NO: 12, or a sequence at least 90% or at least 95% identical thereto.

63. The method of claim 62, wherein the recombinant circular RNA comprises a nucleic acid sequence of SEQ ID NO: 13, or a sequence at least 90% or at least 95% identical thereto.

64. The method of any one of claims 55-57, wherein the at least one reprogramming factor is Nanog, and wherein the Nanog has the amino acid sequence of SEQ ID NO: 14 or 15, or a sequence at least 90% or at least 95% identical thereto.

65. The method of claim 64, wherein the recombinant circular RNA comprises a nucleic acid sequence of SEQ ID NO: 16, or a sequence at least 90% or at least 95% identical thereto.

66. The method of any one of claims 55-57, wherein the at least one reprogramming factor is Lin28A, and wherein the Lin28A has the amino acid sequence of SEQ ID NO: 17, or a sequence at least 90% or at least 95% identical thereto.

67. The method of claim 66, wherein the recombinant circular RNA comprises a nucleic acid sequence of SEQ ID NO: 18, or a sequence at least 90% or at least 95% identical thereto.

68. The method of any one of claims 55-57, wherein the at least one reprogramming factor is c-Myc, and wherein the c-Myc has the amino acid sequence of SEQ ID NO: 19 or 20, or a sequence at least 90% or at least 95% identical thereto.

69. The method of claim 68, wherein the recombinant circular RNA comprises a nucleic acid sequence of SEQ ID NO: 21, or a sequence at least 90% or at least 95% identical thereto.

70. The method of any one of claims 55-57, wherein the at least one reprogramming factor is L-Myc, and wherein the L-Myc has the amino acid sequence of any one of SEQ ID NO: 22-24, or a sequence at least 90% or at least 95% identical thereto.

71. The method of claim 70, wherein the recombinant circular RNA comprises a nucleic acid sequence of SEQ ID NO: 25, or a sequence at least 90% or at least 95% identical thereto.

72. The method of any one of claims 55-57, wherein the circular RNA is substantially non-immunogenic.

73. The method of claim 72, wherein the circular RNA comprises one or more M-6-methyladenosine (m⁶A) residues.

74. The method of any one of claim 55-73, wherein the circular RNA comprises from about 200 nucleotides to about 5,000 nucleotides.

75. The method of any one of claims 55-74, wherein the circular RNA comprises an internal ribosome entry site (IRES) operably linked to the protein-coding sequence.

76. The method of any one of claims 55-75, wherein the circular RNA encodes two or more reprogramming factors selected from Oct3/4, Klf4, Sox2, Nanog, Lin28, c-Myc, or L-Myc.

77. The method of claim 55 or 56, wherein the method comprises contacting the cell with a first circular RNA encoding Oct4 or a fragment or variant thereof, a second circular RNA encoding Sox2 or a fragment or variant thereof, a third circular RNA encoding Klf4 or a fragment or variant thereof, a fourth circular RNA encoding C-Myc or a fragment or variant thereof, a fifth circular RNA encoding Lin28 or a fragment or variant thereof, and a sixth circular RNA encoding Nanog or a fragment or variant thereof.

78. The method of claim 55 or 56, wherein the method comprises contacting the cells with one or more circular RNAs encoding Oct3/4, Klf4, Sox2, Nanog, Lin28, c-Myc, and L-Myc, or fragments or variants thereof.

79. The method of claim any one of claims 55-78, wherein the cell is not contacted with any ancillary factors selected from E3, K3, B18R, or any micro RNAs (miRs).

80. The method of any one of claims 55-79, wherein the cell is directly contacted with the at least one circular RNA.

81. The method of any one of claims 55-80, wherein the cell is contacted with each of the at least one circular RNAs once.

82. The method of any one of claims 55-80, wherein the method comprises contacting the cell with each of the at least one of the circular RNAs two, three, four, or more times.

83. The method of any one of claims 55-80, comprising contacting the cell with each of the at least one circular RNA fewer than four times.

84. The method of any one of claims 55-80, comprising contacting the cell with each of the at least one circular RNAs from 2 to 4 times.

85. The method of any one of claims 55-84, wherein the concentration of the at least one circular RNAs is at least 3 pg RNA/cell.

86. The method of any one of claims 55-85, wherein the concentration of the at least one circular RNAs is from about 5 pg RNA/cell to about 15 pg RNA/cell.

87. The method of any one of claims 55-86, wherein the contacting the cell is performed by electroporation.

88. The method of claim 87, wherein the electroporation uses the Neon® electroporation system.

89. The method of any one of claim 55-78 or 80-88, wherein the method comprises contacting the cell with an RNA polynucleotide encoding one or more viral proteins selected from E3, K3, or B18R.

90. The method of claim 89, wherein:
(a) the B18R has a sequence of SEQ ID NO: 26, or a sequence at least 90% or at least 95% identical thereto;
(b) the E3 has a sequence of SEQ ID NO: 27, or a sequence at least 90% or at least 95% identical thereto; and/or
(c) the K3 has a sequence of SEQ ID NO: 28, or a sequence at least 90% or at least 95% identical thereto.

91. The method of any one of claim 55-78 or 80-90, wherein the method comprises contacting the cell with an microRNA (miR) selected from miR302a, miR302b, miR302c, miR302d, and miR367.

92. The method of any one of claims 55-88, wherein the cell is not contacted with any viral proteins selected from E3, K3, B18R, or any micro RNAs (miRs) selected from miR302a, miR302b, miR302c, miR302d, and miR367.

93. The method of any one of claims 55-82, wherein the enzyme is a transcription activator-like effector nuclease (TALEN), an argonaute endonuclease (NgAgo), a structure-guided endonuclease (SGN), an RNA-guided nuclease (RGN), an Adenosine deaminase acting on RNA (ADAR), or modified or truncated variants thereof.

94. The method of claim 93, wherein the RGN is a Cas nuclease selected from Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, a Cas 14 nuclease or a modified or truncated variant thereof.

95. The method of claim 94, wherein the RGN is a Cas9 nuclease, and the Cas9 nuclease is isolated or derived from S. pyogenes or S. aureus.

96. The method of claim 93, wherein the RGN is selected from any one of APG05083.1, APG07433.1, APG07513.1, APG08290.1, APG05459.1, APG04583.1, and APG1688.1, APG05733.1, APG06207.1, APG01647.1, APG08032.1, APG05712.1, APG01658.1, APG06498.1, APG09106.1, APG09882.1, APG02675.1, APG01405.1, APG06250.1, APG06877.1, APG09053.1, APG04293.1, APG01308.1, APG06646.1, APG09748, APG07433.1, APG00969, APG03128, APG09748, APG00771, APG02789, APG09106, APG02312, APG07386, APG09980, APG05840, APG05241, APG07280, APG09866, and APG00868.

97. The method of any one of claims 55-96, wherein the method further comprises contacting the cell with a guide RNA, or a nucleic acid encoding the same.

98. The method of any one of claim 55 or 57-97, wherein the cell is contacted with the circular RNA before it is contacted with the enzyme or the nucleic acid encoding the same.

99. The method of any one of claim 55 or 87-97, wherein the cell is contacted with the recombinant circular RNA after it is contacted with the enzyme or the nucleic acid encoding the same.

100. The method of any one of claims 55-99, wherein the cell is contacted with an enzyme capable of editing the DNA or RNA of the cell, or a nucleic acid encoding the same, and a guide RNA, or a nucleic acid encoding the same.

101. The method of claim 100, wherein the enzyme is capable of editing the DNA of the cell and wherein the enzyme and the guide RNA are complexed as a ribonucleoprotein prior to contact with the cell.

102. The method of any one of claims 55-101, wherein the contacting the cell is performed by electroporation.

103. A cell generated by the method of any one of claims 55-102.

104. A method for reprogramming a cell, the method comprising contacting a cell with one or more circular RNAs encoding six reprogramming factors consisting of Oct3/4, Klf4, Sox2, Nanog, Lin28, and c-Myc.

105. The method of claim 104, comprising contacting a cell with six circular RNAs each encoding a reprogramming factor from the group consisting of Oct3/4, Klf4, Sox2, Nanog, Lin28, and c-Myc.

106. The method of claim 104 or 105, wherein any one of the circular RNA or linear RNAs are conjugated to a lipid nanoparticle.

107. The method of claim any one of claims 104-106, wherein the cell is not contacted with one or more factors selected from E3, K3, B18R, or one or more micro RNAs (miRs).

108. The method of any one of claim 1-51, 55-102, or 104-107, wherein the circular RNA is exogenous to the cell.

109. A cell generated by the method of any one of claims 104-108.

110. A somatic cell comprising one or more exogenous circular RNAs encoding a reprogramming factor, wherein the reprogramming factor is selected from the group consisting of Oct3/4, Klf4, Sox2, Nanog, Lin28, and c-Myc.

111. The somatic cell of claim 110, wherein the somatic cell comprises one or more exogenous circular RNAs, wherein the one or more circular RNAs each encode a reprogramming factor selected from Oct3/4, Klf4, Sox2, Nanog, Lin28, and c-Myc.

112. The somatic cell of claim 110, wherein the somatic cell comprises six exogenous circular RNAs, wherein each circular RNA encodes one of Oct3/4, Klf4, Sox2, Nanog, Lin28, and c-Myc.

113. The somatic cell of claim 110, wherein the somatic cell comprises five exogenous circular RNAs, wherein each circular RNA encodes one of Oct3/4, Klf4, Sox2, Nanog, and Lin28.

114. The somatic cell of claim 110, wherein the somatic cell comprises four exogenous circular RNAs, wherein each circular RNA encodes one of Oct3/4, Klf4, Sox2, and c-Myc.

115. The somatic cell of claim 110, wherein the somatic cell comprises three exogenous circular RNAs, wherein each circular RNA encodes one of Oct3/4, Klf4, and Sox2.

116. A suspension culture comprising one or more CD34+ cells, wherein the CD34+ cells comprise one or more exogenous circRNAs encoding a reprogramming factor.

117. The suspension culture of claim 116, wherein the CD34+ cells comprise six exogenous circRNAs each encoding one reprogramming factor selected from the group consisting of Oct3/4, Klf4, Sox2, Nanog, Lin28, and c-Myc.

118. The suspension culture of claim 116 or 117, wherein the CD34+ cell does not comprise an exogeneous nucleic acid encoding an ancillary factor selected from E3, K3, B18R, or a micro RNAs (miRs).

119. A composition comprising one or more circular RNAs encoding the reprogramming factors consisting of Oct3/4, Klf4, Sox2, Nanog, Lin28, and c-Myc.

120. A composition comprising two or more circular RNAs encoding the reprogramming factors consisting of Oct3/4, Klf4, Sox2, Nanog, Lin28, and c-Myc.

121. A composition comprising six circular RNAs each encoding one of the reprogramming factors consisting of Oct3/4, Klf4, Sox2, Nanog, Lin28, and c-Myc.

122. A composition comprising five circular RNAs each encoding one of the reprogramming factors consisting of Oct3/4, Klf4, Sox2, Nanog, and Lin28.

123. A composition comprising four circular RNAs each encoding one of the reprogramming factors consisting of Oct3/4, Klf4, Sox2, and c-Myc.

124. A composition comprising three circular RNAs each encoding one of the reprogramming factors consisting of Oct3/4, Klf4, and Sox2.

125. A kit comprising the composition of any one of claims 119-124.

126. A cell comprising the composition of any one of claims 119-124.

127. The cell of claim 126, wherein the cell is a eukaryotic cell.

128. The cell of claim 127, wherein the cell is a mammalian cell.

129. The cell of claim 128, wherein the cell is a human cell.

130. The cell of any one of claims 126-128, wherein the cell is a CD34+ cell, a T cell, or an NK cell.

131. A CD34+ cell comprising one or more circular RNAs encoding one or more reprogramming factors selected from Oct3/4, Klf4, Sox2, Nanog, Lin28, and either one of c-Myc, or L-Myc.

132. The CD34+ cell of claim 131, wherein the reprogramming factors consist of all six of Oct3/4, Klf4, Sox2, Nanog, Lin28, and c-Myc.

133. The CD34+ cell of claim 131, wherein the reprogramming factors consist of all six of Oct3/4, Klf4, Sox2, Nanog, Lin28, and L-Myc.

134. The CD34+ cell of claim 131, wherein the reprogramming factors consist of Oct3/4, Klf4, Sox2, Nanog, and Lin28.

135. The CD34+ cell of claim 131, wherein the reprogramming factors consist of Oct3/4, Klf4, Sox2, and c-Myc.

136. The CD34+ cell of claim 131, wherein the reprogramming factors consist of Oct3/4, Klf4, and Sox2.

137. The CD34+ cell of any one of claims 131-136, wherein the cells exhibit at least one stemness marker selected from SSEA-3, SSEA-4, TRA-1-60, TRA-1-81, TRA-2-49/6E, Alkaline phosphatase, Sox2, E-cadherin, UTF-1, Oct4, Rex1, Nanog, or a combination thereof.

138. The CD34+ cell of any one of claims 131-137, wherein the one or more circular RNAs is exogenous to the cells.

139. The CD34+ cell of any one of claims 131-138, further comprising one or more genetic modifications.

140. The CD34+ cell of claim 139, wherein the one or more genetic modification comprises a gene knockout.

141. The CD34+ cell of claim 139 or 140, wherein the one or more genetic modification comprises a gene knock-in.

142. An induced pluripotent stem cell (iPSC) derived from the CD34+ cell of any one of claims 131-141.

143. The iPSC of claim 142, wherein the cell is hypoimmunogenic.

144. A differentiated cell generated from the iPSC of claim 142 or 143.

145. A method of treating a disease or condition comprising administering to a subject in need thereof the iPSC of claim 142 or 143 or the differentiated cell of claim 126.

146. A method of transdifferentiating a somatic cell comprising contacting the cell with one or more exogenous circular RNAs.

147. A transdifferentiated cell produced by the method of claim 146.

148. A method of differentiating a cell from an induced pluripotent stem cell (iPSC) comprising contacting the iPSC with one or more circular RNAs.

149. A differentiated cell produced by the method of claim 148.


SEQUENCE LISTING (31 sequences)

SEQ ID NO: 1 - protein, 18 aa, unidentified
  REGION 1..18: note = 2A peptide
EGRGSLLTCGDVEENPGP

SEQ ID NO: 2 - protein, 19 aa, unidentified
  REGION 1..19: note = 2A peptide
ATNFSLLKQAGDVEENPGP

SEQ ID NO: 3 - protein, 20 aa, unidentified
  REGION 1..20: note = 2A peptide
QCTNYALLKLAGDVESNPGP

SEQ ID NO: 4 - protein, 22 aa, unidentified
  REGION 1..22: note = 2A peptide
VKQTLNFDLLKLAGDVESNPGP

SEQ ID NO: 5-6 - skipped (2 entries)

SEQ ID NO: 7 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 8 - RNA, 2000 nt, synthetic construct
  source 1..2000: mol_type = other RNA
  misc_feature 1..2000: note = circularized RNA encoding OCT4 reprogramming factor
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaaaaaaacaaaaaacaaaacggctattatgcgttaccggcgagacgctacggactt

SEQ ID NO: 9 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 10 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 11 - RNA, 2357 nt, synthetic construct
  source 1..2357: mol_type = other RNA
  misc_feature 1..2357: note = circularized RNA encoding KLF4 reprogramming factor
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaaaaaaacaaaaaacaaaacggctattatgcgttaccggcgagacgctacggactt

SEQ ID NO: 12 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 13 - RNA, 1871 nt, synthetic construct
  source 1..1871: mol_type = other RNA
  misc_feature 1..1871: note = circularized RNA encoding SOX2 reprogramming factor
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgaaaaaaacaaaaaacaaaacggctattatgcgttaccggcgagacgctacggactt

SEQ ID NO: 14 - protein, 289 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 15 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 16 - RNA, 1835 nt, synthetic construct
  source 1..1835: mol_type = other RNA
  misc_feature 1..1835: note = circularized RNA encoding NANOG reprogramming factor
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaaaaaaacaaaaaacaaaacggctattatgcgttaccggcgagacgctacggactt

SEQ ID NO: 17 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 18 - RNA, 1547 nt, synthetic construct
  source 1..1547: mol_type = other RNA
  misc_feature 1..1547: note = circularized RNA encoding LIN28 reprogramming factor
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgaaaaaaacaaaaaacaaaacggctattatgcgttaccggcgagacgctacggactt

SEQ ID NO: 19 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 20 - protein, 453 aa, Homo sapiens
MDFFRVVENQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 21 - RNA, 2237 nt, synthetic construct
  source 1..2237: mol_type = other RNA
  misc_feature 1..2237: note = circularized RNA encoding cMYC reprogramming factor
aaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaaaaaaacaaaaaacaaaacggctattatgcgttaccggcgagacgctacggactt

SEQ ID NO: 22 - protein, 236 aa, Homo sapiens
MCVCAGCRAAPSRRGAGPLQVAGGWSEGADMDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSGKDLPEPSKRGPPHGWPKLCPCLRSGIGSSQALGPSPPLFG

SEQ ID NO: 23 - protein, 364 aa, Homo sapiens
MDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 24 - protein, 394 aa, Homo sapiens
MCVCAGCRAAPSRRGAGPLQVAGGWSEGADMDYDSYQHYFYDYDCGEDFYRSTAPSEDIWKKFELVPSPPTSPPWGLGPGAGDPAPGIGPPEPWPGGCTGDEAESRGHSKGWGRNYASIIRRDCMWSGFSARERLERAVSDRLAPGAPRGNPPKASAAPDCTPSLEAGNPAPAAPCPLGEPKTQACSGSESPSDSENEEIDVVTVEKRQSLGIRKPVTITVRADPLDPCMKHFHISIHQQQHNYAARFPPESCSQEEASERGPQEEVLERDAAGEKEDEEDEEIVSPPPVESEAAQSCHPKPVSSDTEDVTKRKNHNFLERKRRNDLRSRFLALRDQVPTLASCSKAPKVVILSKALEYLQALVGAEKRMATEKRQLRCRQQQLQKRIAYLTGY

SEQ ID NO: 25 - RNA, 1185 nt, synthetic construct
  source 1..1185: mol_type = other RNA
  misc_feature 1..1185: note = circularized RNA encoding L-Myc reprogramming factor
atgtgcgtgtgtgctggctgccgggctgccccgagccggcggggagccggtccgctccaggtggcgggcggctggagcgagggagcggacatggactacgactcgtaccagcactatttctacgactatgactgcggggaggatttctaccgctccacggcgcccagcgaggacatctggaagaaattcgagctggtgccatcgccccccacgtcgccgccctggggcttgggtcccggcgcaggggacccggcccccgggattggtcccccggagccgtggcccggagggtgcaccggagacgaagcggaatcccggggccactcgaaaggctggggcaggaactacgcctccatcatacgccgtgactgcatgtggagcggcttctcggcccgggaacggctggagagagctgtgagcgaccggctcgctcctggcgcgccccgggggaacccgcccaaggcgtccgccgccccggactgcactcccagcctcgaagccggcaacccggcgcccgccgccccctgtccgctgggcgaacccaagacccaggcctgctccgggtccgagagcccaagcgactcggagaatgaagaaattgatgttgtgacagtagagaagaggcagtctctgggtattcggaagccggtcaccatcacggtgcgagcagaccccctggatccctgcatgaagcatttccacatctccatccatcagcaacagcacaactatgctgcccgttttcctccagaaagctgctcccaagaagaggcttcagagaggggtccccaagaagaggttctggagagagatgctgcaggggaaaaggaagatgaggaggatgaagagattgtgagtcccccacctgtagaaagtgaggctgcccagtcctgccaccccaaacctgtcagttctgatactgaggatgtgaccaagaggaagaatcacaacttcctggagcgcaagaggcggaatgacctgcgttcgcgattcttggcgctgagggaccaggtgcccaccctggccagctgctccaaggcccccaaagtagtgatcctaagcaaggccttggaatacttgcaagccctggtgggggctgagaagaggatggctacagagaaaagacagctccgatgccggcagcagcagttgcagaaaagaattgcatacctcactggctactaa

SEQ ID NO: 26 - protein, 351 aa, Vaccinia virus
  REGION 1..351: note = B18R protein
MTMKMMVHIYFVSLLLLLFHSYAIDIENEITEFFNKMRDTLPAKDSKWLNPACMFGGTMNDIAALGEPFSAKCPPIEDSLLSHRYKDYVVKWERLEKNRRRQVSNKRVKHGDLWIANYTSKFSNRRYLCTVTTKNGDCVQGIVRSHIRKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 27 - protein, 190 aa, Vaccinia virus
  REGION 1..190: note = E3 protein
MSKIYIDERSNAEIVCEAIKTIGIEGATAAQLTRQLNMEKREVNKALYDLQRSAMVYSSDDIPPRWFMTTEADEADADAMSDVIIDDVSREKSMREDHKSFDDVIPAKKIIDWKGANPVTVINEYCQITRRDWSFRIESVGPSNSPTFYACVDIDGRVFDKADGKSKRDAKNNAAKLAVDKLLGYVIIRF

SEQ ID NO: 28 - protein, 88 aa, Vaccinia virus
  REGION 1..88: note = K3 protein
MLAFCYSLPNAGDVIKGRVYEKDYALYIYLFDYPHFEAILAESVKMHMDRYVEYRDKLVGKTVKVKVIRVDYTKGYIDVNYKRMCRHQ

SEQ ID NO: 29 - RNA, 320 nt, synthetic construct
  source 1..320: mol_type = other RNA
  misc_feature 1..320: note = Circularized human BIRC6 mRNA
ctaaaccaggtggacaggtgaaatgtcagtatatctctgctgtggataaagttatatttgtggatgattatgcagtagggtgtaggaaggaccttaatggaatcttgttgttagacactgctctgcaaactccagtttcaaagcaggatgatgtggttcagcttgaattacccgttacagaggcacagcagctcttatcagcatgtttagaaaaggtagatatttctagtacagagggttatgatttgttcatcacacagctcaaagatggtttaaaaaatacatctcatgagactgcagcaaaccacaaagttgctaag

SEQ ID NO: 30 - RNA, 251 nt, synthetic construct
  source 1..251: mol_type = other RNA
  misc_feature 1..251: note = Circularized human CORO1C mRNA
aaaaatatgcaggaaccaattgctcttcatgagatggacactagcaatggggtgttgctgcctttctatgaccctgacaccagcatcatttacttatgtggaaagggtgacagcagtattcgctattttgagatcacggatgaatccccgtacgtccactacctcaacacattcagcagcaaggagcctcagagagggatgggttacatgcccaagaggggacttgatgttaacaaatgtgagattgccag

SEQ ID NO: 31 - RNA, 353 nt, synthetic construct
  source 1..353: mol_type = other RNA
  misc_feature 1..353: note = Circularized human MAN1A2 mRNA
ggaagaggaagaacgtctgagaaataaaattcgagctgatcatgagaaggccttggaagaagcaaaagaaaaattaagaaagtcaagagaggaaattcgagcagaaattcagacagagaaaaataaggtagtccaagaaatgaagataaaagagaacaagccactgccaccagtccctattcccaaccttgtaggaatacgtggtggagacccagaagataatgacataagagagaaaagggaaaaaattaaagagatgatgaaacatgcttgggataactataggacatatgggtggggacataatgaactcagacctattgcaaggaaaggacactcccctaacatatttg